(19) United States
(12) Patent Application Publication
(10) Pub. No.: US 2025/0144149 A1
(43) Pub. Date: May 8, 2025

(54) ADENOSINE DEAMINASE BASE EDITORS AND METHODS FOR USE THEREOF

(71) Applicant: Beam Therapeutics Inc., Cambridge, MA (US)

(72) Inventors: Nicole Gaudelli, Cambridge, MA (US); Tanggis Bohnuud, Cambridge, MA (US); Scott Haihua Chu, Cambridge, MA (US)

(73) Assignee: Beam Therapeutics Inc., Cambridge, MA (US)

(21) Appl. No.: 18/984,585

(22) Filed: Dec. 17, 2024

Related U.S. Application Data
(63) Continuation of application No. PCT/US2023/069187, filed on Jun. 27, 2023.
(60) Provisional application No. 63/355,980, filed on Jun. 27, 2022.

(51) Int. Cl. A61K 35/28; A61K 38/42; C12N 9/22; C12N 9/80; C12N 15/11; C12N 15/85

(52) U.S. Cl. CPC A61K 35/28; A61K 38/42; C12N 9/22; C12N 9/80; C12N 15/111; C12N 15/85; C07K 2319/80; C12N 2310/20; C12Y 305/04004

(57) ABSTRACT

Adenosine deaminase base editors, compositions comprising the same, and methods for use thereof for altering a target nucleobase in a polynucleotide sequence. In embodiments of the disclosure, the base editors of the disclosure may be used to treat a disease or disorder, such as a hemoglobinopathy (e.g., sickle cell disease (SCD)).

### CROSS REFERENCE TO RELATED APPLICATIONS

This application is a continuation under 35 U.S.C. § 111(a) of PCT International Patent Application No. PCT/US2023/069187, filed Jun. 27, 2023, designating the United States and published in English, which claims priority to U.S. Provisional Application No. 63/355,980, filed Jun. 27, 2022, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Jan. 16, 2025, is named 180802-043903US_SL.xml and is 1,111,690 bytes in size.

### BACKGROUND

Targeted editing of nucleic acid sequences (e.g., the targeted cleavage or the targeted introduction of a specific modification into genomic DNA) is a highly promising approach for the study of gene function and also has the potential to provide new therapies for human genetic diseases. Currently available base editors include cytidine base editors (e.g., BE4) that convert target C•G base pairs to T•A and adenine base editors (e.g., ABE7.10) that convert A•T to G•C. There is a need in the art for improved base editors capable of inducing modifications within a target sequence.

### SUMMARY

As described below, the disclosure features adenosine deaminase base editors, compositions comprising the same, and methods for use thereof for altering a target nucleobase in a polynucleotide sequence. In some embodiments, the base editors of the disclosure may be used to treat a disease or disorder, such as a hemoglobinopathy (e.g., sickle cell disease (SCD)).

In one aspect, the disclosure features a method for treating a hemoglobinopathy in a subject in need thereof. The method involves a) contacting an isolated hematopoietic stem cell or progenitor thereof with two or more guide polynucleotides, or one or more polynucleotides encoding the guide polynucleotides, and a base editor containing a nucleic acid programmable DNA binding protein (napDNAbp) and an adenosine deaminase, or one or more polynucleotides encoding the base editor. The adenosine deaminase contains a combination of alterations relative to the following sequence of TadA*7.10 and has at least 85% sequence identity to the following sequence of TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1). The combination of alterations contains i) F149Y and V82T; or ii) F149Y, T166I, and/or D167N, and the alteration V82T. The two or more guide polynucleotides target the base editor to effect an alteration to a beta globin polynucleotide (HBB) that results in expression of a beta globin polypeptide having an alanine at position 6 (Hb G-Makassar). The two or more guide polynucleotides target the base editor to effect an alteration in a cluster of differentiation (CD117) polynucleotide that results in expression of a CD117 polypeptide with reduced binding to an antibody that selectively binds a wild type CD117 polypeptide, thereby generating an edited cell. The method also involves (b) where the cell is in the subject or the method further involves administering the edited cell to the subject.

In another aspect, the disclosure features a cell produced by the method of any aspect of the disclosure, or embodiments thereof.

In another aspect, the disclosure features a pharmaceutical composition containing an effective amount of the cell of any aspect of the disclosure, or embodiments thereof.

In another aspect, the disclosure features an adenosine deaminase polypeptide containing a combination of alterations relative to the following sequence of TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1). The combination of alterations contain F149Y and V82T. The adenosine deaminase has at least 85% sequence identity to TadA*7.10.

In another aspect, the disclosure features an adenosine deaminase polypeptide containing the following amino acid sequence:

(SEQ ID NO: 518)
SEVEFSHEYWMRHALTLAKRARDEREVPVGAVLVLNNRVIGEGWNRAIGL
HDPTAHAEIMALRQGGLVMQNYRLYDATLYTTFEPCVMCAGAMIHSRIGR
VVFGVRNAKTGAAGSLMDVLHHPGMNHRVEITEGILADECAALLCRFYRM
PRRVFNAQKKAQSSTD.

In another aspect, the disclosure features an adenosine deaminase polypeptide containing a combination of alterations to the following sequence of TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1). The combination of alterations contains F149Y, T166I, and/or D167N, and the alteration V82T. The adenosine deaminase has at least 85% sequence identity to TadA*7.10.

In another aspect, the disclosure features an adenosine deaminase polypeptide containing the following amino acid sequence:

(SEQ ID NO: 519)
SEVEFSHEYWMRHALTLAKRARDEREVPVGAVLVLNNRVIGEGWNRAIGL
HDPTAHAEIMALRQGGLVMQNYRLYDATLYTTFEPCVMCAGAMIHSRIGR
VVFGVRNAKTGAAGSLMDVLHHPGMNHRVEITEGILADECAALLCDFYRM
PRRVFNAQKKAQSSIN.

In another aspect, the disclosure features base editor containing a nucleic acid programmable DNA binding protein (napDNAbp) and the adenosine deaminase of any aspect of the disclosure, or embodiments thereof.

In another aspect, the disclosure features a base editor containing: a nucleic acid programmable DNA binding protein (napDNAbp) and an adenosine deaminase. The adenosine deaminase contains a combination of alterations relative to TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1). The combination of alterations contains I76Y, V82T, Y123H, Y147R, F149Y, and Q154R. The adenosine deaminase has at least 85% sequence identity to TadA*7.10.

In another aspect, the disclosure features base editor containing a nucleic acid programmable DNA binding protein (napDNAbp) and an adenosine deaminase. The adenosine deaminase contains a combination of alterations relative to TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1). The combination of alterations contains I76Y, V82T, Y123H, Y147D, F149Y, Q154R, T166I, and D167N. The adenosine deaminase has at least 85% sequence identity to TadA*7.10.

In another aspect, the disclosure features polynucleotide encoding the adenosine deaminase polypeptide or base editor of any aspect of the disclosure or embodiments thereof.

In another aspect, the disclosure features method of altering a nucleobase of a polynucleotide. The method involves contacting the polynucleotide with the base editor of any aspect of the disclosure, or embodiments thereof, thereby altering a nucleobase of the polynucleotide.

In another aspect, the disclosure features a base editor system containing the base editor of any aspect of the disclosure, or embodiments thereof, or one or more polynucleotides encoding the base editor, and a guide polynucleotide, or a polynucleotide encoding the guide polynucleotide, that targets the base editor to effect an alteration to a polynucleotide associated with a genetic disorder.

In another aspect, the disclosure features a base editor system containing the base editor of any aspect of the disclosure, or embodiments thereof, or one or more polynucleotides encoding the base editor, and two or more guide polynucleotides, or one or more polynucleotides encoding the two or more guide polynucleotides. One of the guide polynucleotides targets the base editor to effect an alteration to a beta globin polynucleotide (HBB) that results in expression of a beta globin polypeptide having an alanine at position 6 (Hb G-Makassar). Another of the guide polynucleotides targets the base editor to effect an alteration in a cluster of differentiation (CD117) polynucleotide that results in expression of a CD117 polypeptide with reduced binding to an antibody that selectively binds a wild type CD117 polypeptide.

In another aspect, the disclosure features kit suitable for use in the method of any one of the above claims, where the kit contains the base editor of any aspect of the disclosure, or embodiments thereof, or a polynucleotide encoding the base editor.

In any aspect of the disclosure, or embodiments thereof, the combination of alterations further contains an alteration selected from one or more of I76Y, Y123H, Y147R, and Q154R. In any aspect of the disclosure, or embodiments thereof, the combination of alterations contains I76Y, V82T, Y123H, Y147R, F149Y, and Q154R. In any aspect of the disclosure, or embodiments thereof, the adenosine deaminase has at least 90% or 95% identity to TadA*7.10. In any aspect of the disclosure, or embodiments thereof, the adenosine deaminase contains the following amino acid sequence: SEVEFSHEYWMRHALTLAKRARDEREVPVGAVLVLNNRVIGEGWNRAIGLHDPTAHAEIMAL RQGGLVMQNYRLYDATLYTTFEPCVMCAGAMIHSRIGRVVFGVRNAKTGAAGSLMDVLHHPG MNHRVEITEGILADECAALLCRFYRMPRRVFNAQKKAQSSTD (SEQ ID NO: 518). In any aspect of the disclosure, or embodiments thereof, the combination of alterations contains F149Y, T166I, and D167N. In any aspect of the disclosure, or embodiments thereof, the combination of alterations further contains the following additional alterations: I76Y, Y123H, Y147D, and Q154R. In any aspect of the disclosure, or embodiments thereof, the combination of alterations contains I76Y, V82T, Y123H, Y147D, F149Y, Q154R, T166I, and D167N. In any aspect of the disclosure, or embodiments thereof, the adenosine deaminase contains the following amino acid sequence:

(SEQ ID NO: 519)
SEVEFSHEYWMRHALTLAKRARDEREVPVGAVLVLNNRVIGEGWNRAIGL
HDPTAHAEIMALRQGGLVMQNYRLYDATLYTTFEPCVMCAGAMIHSRIGR
VVFGVRNAKTGAAGSLMDVLHHPGMNHRVEITEGILADECAALLCDFYRM
PRRVFNAQKKAQSSIN.

In any aspect of the disclosure, or embodiments thereof, the two or more guide polynucleotides contain a spacer nucleotide sequence selected from one or more of: UUCUCCACAGGAGUCAGGUG (SEQ ID NO: 445); ACUUCUCCACAGGAGUCAGG (SEQ ID NO: 446); GACUUCUCCACAGGAGUCAGG (SEQ ID NO: 447); CUUCUCCACAGGAGUCAGG (SEQ ID NO: 448); CUUCUCCACAGGAGUCAGAU (SEQ ID NO: 449); ACUUCUCCACAGGAGUCAGAU (SEQ ID NO: 450); GACUUCUCCACAGGAGUCAGAU (SEQ ID NO: 451); UCUGACUCCUGUGGAGAAGUCU (SEQ ID NO: 452); AGACUUCUCCACAGGAGUCAGA (SEQ ID NO: 453); and UCCACAGGAGUCAGAUGCAC (SEQ ID NO: 454). In any aspect of the disclosure, or embodiments thereof, the two or more guide polynucleotides contain a spacer containing the following sequence UUCUCCACAGGAGUCAGGUG (SEQ ID NO: 445). In any aspect of the disclosure, or embodiments thereof, the two or more guide polynucleotides contain a scaffold with the following nucleotide sequence: GUUUUAGAGCUAGAAAUAGCAAGUUAAAAUAAGGCUAGUCCGUUAUCAACUUGAAAAAGUGG CACCGAGUCGGUGCUUUU (SEQ ID NO: 317). In any aspect of the disclosure, or embodiments thereof, the two or more guide polynucleotides contain a spacer nucleotide sequence selected from those listed in Table 2. In any aspect of the disclosure, or embodiments thereof, the two or more guide polynucleotides contain a spacer nucleotide sequence selected from one or more of:

(SEQ ID NO: 660; CC128)
AAAUAUAAUAGCUGGCAUCA;
(SEQ ID NO: 722; CC200)
AUAAUAGCUGGCAUCACGGU;
(SEQ ID NO: 723; gRNA889)
CCACUAGCUUUCCAAACGGU;
(SEQ ID NO: 724; gRNA908)
GCUGAACUGAUAGUCCACGU;
(SEQ ID NO: 725; gRNA918)
UUUGACAAAGCCCGGAUCAG;
(SEQ ID NO: 726; gRNA923)
UGAAAGUGAGGCCAGGUACU;
(SEQ ID NO: 727; gRNA928)
AAACAGUCAGGUGAGUGAAU;
(SEQ ID ON: 728; gRNA929)
AACUACAGGAGAAAUAUAAU;
and
(SEQ ID NO: 729; gRNA944)
GAUUAAAAGGCACCGAAGGA.

In any aspect of the disclosure, or embodiments thereof, the two or more guide polynucleotides contain a scaffold with a sequence selected from GUUUUAGAGCUAGAAAUAGCAAGUUAAAAUAAGGCUAGUCCGUUAUCAACUUGAAAAAGUGG CACCGAGUCGGUGCmUsmUsmUsU (SEQ ID NO: 521); and GUUUUAGAmGmCmCmGmGmCmGmGmAmAmAmCmGmCmCmGmGmCAAGUUAAAAUAAGGCUAG UCCGUUAmUmCAAmCmUmUGGACUUCGGUCCmAmAmGUGGmCmAmCmCmGmAmGmUmCmGmG mUmGmCmUsmUsmUsmU (SEQ ID NO: 522), where “N” represents any nucleotide, “mN” indicates a 2′-OMe modification of the nucleotide “N”, and “Ns” indicates that the nucleotide “N” is linked to the following nucleotide by a phosphorothioate (PS). In any aspect of the disclosure, or embodiments thereof, the two or more guide polynucleotides contain a polynucleotide sequence selected from mNsmNsmNsNNNNNNNNNNNNNNNNNGUUUUAGAGCUAGAAAUAGCAAGUUAAAAUAAGGCU AGUCCGUUAUCAACUUGAAAAAGUGGCACCGAGUCGGUGCmUsmUsmUsU (SEQ ID NO: 443); and mNsmNsmNsNNNNNNNNNNNNNNNNNGUUUUAGAmGmCmCmGmGmCmGmGmAmAmAmCmGmC mCmGmGmCAAGUUAAAAUAAGGCUAGUCCGUUAmUmCAAmCmUmUGGACUUCGGUCCmAmAm GUGGmCmAmCmCmGmAmGmUmCmGmGmUmGmCmUsmUsmUsmU (SEQ ID NO: 444), where “N” represents any nucleotide, “mN” indicates a 2′-OMe modification of the nucleotide “N”, and “Ns” indicates that the nucleotide “N” is linked to the following nucleotide by a phosphorothioate (PS).

In any aspect of the disclosure, or embodiments thereof, the napDNAbp contains a variant of SpCas9 having an altered protospacer-adjacent motif (PAM) specificity. In some embodiments, the altered PAM has specificity for the nucleic acid sequence 5′-NGC-3′. In any aspect of the disclosure, or embodiments thereof, the napDNAbp recognizes an NRCH PAM sequence, where R is A or G, and H is A, C, or T. In any aspect of the disclosure, or embodiments thereof, the napDNAbp recognizes the PAM nucleotide sequence CACC. In any aspect of the disclosure, or embodiments thereof, the napDNAbp has at least 85% amino acid sequence identity to the following amino acid sequence: DKKYSIGLAIGTNSVGWAVITDEYKVPSKKFKVLGNTDRHSIKKNLIGALLFDSGETAEATR LKRTARRRYTRRKNRICYLQEIFSNEMAKVDDSFFHRLEESFLVEEDKKHERHPIFGNIVDE VAYHEKYPTIYHLRKKLVDSTDKADLRLIYLALAHMIKFRGHFLIEGDLNPDNSDVDKLFIQ LVQTYNQLFEENPINASGVDAKAILSARLSKSRRLENLIAQLPGEKKNGLFGNLIALSLGLT PNFKSNFDLAEDAKLQLSKDTYDDDLDNLLAQIGDQYADLFLAAKNLSDAILLSDILRVNTE ITKAPLSASMVKRYDEHHQDLTLLKALVRQQLPEKYKEIFFDQSKNGYAGYIDGGASQEEFY KFIKPILEKMDGTEELLVKLNREDLLRKQRTFDNGTIPHQIHLGELHAILRRQGDFYPFLKD NREKIEKILTFRIPYYVGPLARGNSRFAWMTRKSEETITPWNFEEVVDKGASAQSFIERMTN FDKNLPNEKVLPKHSLLYEYFTVYNELTKVKYVTEGMRKPAFLSGEQKKAIVDLLFKTNRKV TVKQLKEDYFKKIECFDSVEISGVEDRFNASLGTYHDLLKIIKDKDFLDNEENEDILEDIVL TLTLFEDREMIEERLKTYAHLFDDKVMKQLKRLRYTGWGRLSRKLINGIRDKQSGKTILDFL KSDGFANRNFMQLIHDDSLTFKEDIQKAQVSGQGDSLHEHIANLAGSPAIKKGILQTVKVVD ELVKVMGGHKPENIVIEMARENQTTQKGQKNSRERMKRIEEGIKELGSQILKEHPVENTQLQ NEKLYLYYLQNGRDMYVDQELDINRLSDYDVDHIVPQSFLKDDSIDNKVLTRSDKNRGKSDN VPSEEVVKKMKNYWRQLLNAKLITQRKFDNLTKAERGGLSELDKAGFIKRQLVETRQITKHV AQILDSRMNTKYDENDKLIREVKVITLKSKLVSDFRKDFQFYKVREINNYHHAHDAYLNAVV GTALIKKYPKLESEFVYGDYKVYDVRKMIAKSEQEIGKATAKYFFYSNIMNFFKTEITLANG EIRKRPLIETNGETGEIVWDKGRDFATVRKVLSMPQVNIVKKTEVQTGGFSKESILPKGNSD KLIARKKDWDPKKYGGFNSPTVAYSVLVVAKVEKGKSKKLKSVKELLGITIMERSSFEKNPI DFLEAKGYKEVKKDLIIKLPKYSLFELENGRKRMLASAGVLQKGNELALPSKYVNFLYLASH YEKLKGSPEDNEQKQLFVEQHKHYLDEIIEQISEFSKRVILADANLDKVLSAYNKHRDKPIR EQAENIIHLFTLTNLGAPAAFKYFDTTINRKQYNTTKEVLDATLIRQSITGLYETRIDLSQL G (SEQ ID NO: 520), and recognizes a CACC PAM sequence.

In any aspect of the disclosure, or embodiments thereof, the hematopoietic stem cell or progenitor thereof is autologous to the subject. In any aspect of the disclosure, or embodiments thereof, the hematopoietic stem cell or progenitor thereof is allogeneic to the subject. In any aspect of the disclosure, or embodiments thereof, the contacting is in vitro and the method involves administering the edited cell to the subject. In any aspect of the disclosure, or embodiments thereof, the subject is a mammal.

In any aspect of the disclosure, or embodiments thereof, the hemoglobinopathy is selected from one or more of sickle cell anemia, thalassemia, Fanconi anemia, aplastic anemia, and Wiskott-Aldrich syndrome.

In any aspect of the disclosure, or embodiments thereof, the deaminase is a monomer or heterodimer. In any aspect of the disclosure, or embodiments thereof, the base editor polypeptide is an internal base editor (IBE) containing the deaminase inserted at an internal location of the napDNAbp.

In any aspect of the disclosure, or embodiments thereof, the deaminase is fused to the napDNAbp or forms a complex with the napDNAbp. In any aspect of the disclosure, or embodiments thereof, the napDNAbp is a nuclease inactive or nickase variant.

In any aspect of the disclosure, or embodiments thereof, a) one of the guide polynucleotides contains a spacer sequence selected from one or more of: UUCUCCACAGGAGUCAGGUG (SEQ ID NO: 445); ACUUCUCCACAGGAGUCAGG (SEQ ID NO: 446); GACUUCUCCACAGGAGUCAGG (SEQ ID NO: 447); CUUCUCCACAGGAGUCAGG (SEQ ID NO: 448); CUUCUCCACAGGAGUCAGAU (SEQ ID NO: 449); ACUUCUCCACAGGAGUCAGAU (SEQ ID NO: 450); GACUUCUCCACAGGAGUCAGAU (SEQ ID NO: 451); UCUGACUCCUGUGGAGAAGUCU (SEQ ID NO: 452); AGACUUCUCCACAGGAGUCAGA (SEQ ID NO: 453); and UCCACAGGAGUCAGAUGCAC (SEQ ID NO: 454); and b) another of guide polynucleotides contains a spacer sequence selected from one or more of

(SEQ ID NO: 660; CC128)
AAAUAUAAUAGCUGGCAUCA;
(SEQ ID NO: 722; CC200)
AUAAUAGCUGGCAUCACGGU;
(SEQ ID NO: 723; gRNA889)
CCACUAGCUUUCCAAACGGU;
(SEQ ID NO: 724; gRNA908)
GCUGAACUGAUAGUCAACGU;
(SEQ ID NO: 725; gRNA918)
UUUGACAAAGCCCGGAUCAG;
(SEQ ID NO: 726; gRNA923)
UGAAAGUGAGGCCAGGUACU;
(SEQ ID NO: 727; gRNA928)
AAACAGUCAGGUGAGUGAAU;
(SEQ ID NO: 728; gRNA929)
AACUACAGGAGAAAUAUAAU;
and
(SEQ ID NO: 729; gRNA944)
GAUUAAAAGGCACCGAAGGA.

In any aspect of the disclosure, or embodiments thereof, the base editor contains the following amino acid sequence:

(SEQ ID NO: 524)
MSEVEFSHEYWMRHALTLAKRARDEREVPVGAVLVLNNRVIGEGWNRAIG
LHDPTAHAEIMALRQGGLVMQNYRLYDATLYTTFEPCVMCAGAMIHSRIG
RVVFGVRNAKTGAAGSLMDVLHHPGMNHRVEITEGILADECAALLCRFYR
MPRRVFNAQKKAQSSTDSGGSSGGSSGSETPGTSESATPESSGGSSGGSD
KKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILL
SDILRVNTEITKAPLSASMVKRYDEHHQDLTLLKALVRQQLPEKYKEIFF
DQSKNGYAGYIDGGASQEEFYKFIKPILEKMDGTEELLVKLNREDLLRKQ
RTFDNGIIPHQIHLGELHAILRRQGDFYPFLKDNREKIEKILTFRIPYYV
GPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLK
RLRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMG
GHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIRE
VKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKGNSDKLIARKKDWDPKKYGGFNSPTVAYSVLVVAKVEKG
KSKKLKSVKELLGITIMERSSFEKNPIDFLEAKGYKEVKKDLIIKLPKYS
LFELENGRKRMLASAGVLQKGNELALPSKYVNFLYLASHYEKLKGSPEDN
EQKQLFVEQHKHYLDEIIEQISEFSKRVILADANLDKVLSAYNKHRDKPI
REQAENIIHLFTLTNLGAPAAFKYFDTTINRKQYNTTKEVLDATLIRQSI
TGLYETRIDLSQLGGDEGADKRTADGSEFESPKKKRKV.

In any aspect of the disclosure, or embodiments thereof, the base editor contains the following amino acid sequence:

(SEQ ID NO: 525)
MSEVEFSHEYWMRHALTLAKRARDEREVPVGAVLVLNNRVIGEGWNRAIG
LHDPTAHAEIMALRQGGLVMQNYRLYDATLYTTFEPCVMCAGAMIHSRIG
RVVFGVRNAKTGAAGSLMDVLHHPGMNHRVEITEGILADECAALLCDFYR
MPRRVFNAQKKAQSSINSGGSSGGSSGSETPGTSESATPESSGGSSGGSD
KKYSIGLAIGTNSVGWAVITDEYKVPSKKFKVLGNTDRHSIKKNLIGALL
FDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLR
LIYLALAHMIKFRGHFLIEGDLNPDNSDVDKLFIQLVQTYNQLFEENPIN
ASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILL
SDILRVNTEITKAPLSASMVKRYDEHHQDLTLLKALVRQQLPEKYKEIFF
DQSKNGYAGYIDGGASQEEFYKFIKPILEKMDGTEELLVKLNREDLLRKQ
RTFDNGIIPHQIHLGELHAILRRQGDFYPFLKDNREKIEKILTFRIPYYV
GPLARGNSRFAWMTRKSEETITPWNFEEVVDKGASAQSFIERMTNFDKNL
PNEKVLPKHSLLYEYFTVYNELTKVKYVTEGMRKPAFLSGEQKKAIVDLL
FKTNRKVTVKQLKEDYFKKIECFDSVEISGVEDRFNASLGTYHDLLKIIK
DKDFLDNEENEDILEDIVLTLTLFEDREMIEERLKTYAHLFDDKVMKQLK
RLRYTGWGRLSRKLINGIRDKQSGKTILDFLKSDGFANRNFMQLIHDDSL
TFKEDIQKAQVSGQGDSLHEHIANLAGSPAIKKGILQTVKVVDELVKVMG
GHKPENIVIEMARENQTTQKGQKNSRERMKRIEEGIKELGSQILKEHPVE
NTQLQNEKLYLYYLQNGRDMYVDQELDINRLSDYDVDHIVPQSFLKDDSI
DNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIRE
VKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKGNSDKLIARKKDWDPKKYGGFNSPTVAYSVLVVAKVEKG
KSKKLKSVKELLGITIMERSSFEKNPIDFLEAKGYKEVKKDLIIKLPKYS
LFELENGRKRMLASAGVLQKGNELALPSKYVNFLYLASHYEKLKGSPEDN
EQKQLFVEQHKHYLDEIIEQISEFSKRVILADANLDKVLSAYNKHRDKPI
REQAENIIHLFTLTNLGAPAAFKYFDTTINRKQYNTTKEVLDATLIRQSI
TGLYETRIDLSQLGGDEGADKRTADGSEFESPKKKRKV.

In any aspect provided herein, or embodiments thereof, the method is not a process for modifying the germline genetic identity of human beings.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “ABTx052” or “mAb-7” is meant an antibody having at least about 85% amino acid sequence identity to an antibody sequence of antibody ABTx052 or comprising VH and/or VL CDRs 1-3 of ABTx052 or antigen binding fragments thereof, wherein each of the antibody, CDRs, and antigen binding fragments specifically bind to a wild type CD117 polypeptide but fail to detectably bind or have only reduced binding to an altered CD117 polypeptide. In embodiments, the antibody or antigen binding fragment thereof has at least 90%, 93%, 95%, 98%, 99% or 100% amino acid sequence identity to an antibody sequence of antibody ABTx052. In embodiments, ABTx052 selectively binds a wild type CD117 polypeptide. Exemplary heavy chain and light chain sequences for antibody ABTx052 are provided below, where embodiments of the variable regions are in plain text, embodiments of the constant domains are in bold, and embodiments of complementarity determining regions (CDRs), i.e., CDR1, CDR2, and CDR2, are underlined:

ABTx052 heavy chain (HC):
(SEQ ID NO: 419)
QVQLVQSGAAVKKPGESLKISCKGSGYRFTSYWIGWVRQMPGKGLEWMGI
IYPGDSDTRYSPSFQGQVTISAGKSISTAYLQWSSLKASDTAMYYCARHG
RGYNGYEGAFDIWGQGTMVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTT
PPVLDSDGSFFLYSKLTVDKSRWQQGNVFSCSVMHEALHNHYTQKSLSLS
PG
ABTx052 light chain (LC):
(SEQ ID NO: 420)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

The three CDRs of the ABTx052 antibody VH region are as follows:

VH CDR1:
(SEQ ID NO: 421)
GYRFTSYW;
VH CDR2:
(SEQ ID NO: 422)
IYPGDSDT
or
(SEQ ID NO: 958)
IYPGDSDTR;
and
VH CDR3:
(SEQ ID NO: 423)
ARHGRGYNGYEGAFDI.

The three CDRs of the ABTx052 antibody VL region are as follows:

VL CDR1:
(SEQ ID NO: 424)
QGISSA;
VL CDR2:
DAS;
and
VL CDR3:
(SEQ ID NO: 425)
QQFNSYPLT.

The four framework (FR) regions, i.e., FR1, FR2, FR3, and FR4, of the ABTx052 antibody are located on either side of each of the CDRs in VH and VL region sequences shown supra. In particular, the four FRs of the ABTx052 antibody VH region are as follows:

VH FR1:
(SEQ ID NO: 426)
QVQLVQSGAAVKKPGESLKISCKGS;
VH FR2:
(SEQ ID NO: 427)
IGWVRQMPGKGLEWMGI;
and
VH FR3:
(SEQ ID NO: 428)
RYSPSFQGQVTISAGKSISTAYLQWSSLKASDTAMYYC
or
(SEQ ID NO: 973)
YSPSFQGQVTISAGKSISTAYLQWSSLKASDTAMYYC;
VH FR4:
(SEQ ID NO: 429)
WGQGTMVTVSS.

The four FRs of the ABTx052 antibody VL region are as follows:

(SEQ ID NO: 433)
VL FR1:
(SEQ ID NO: 430)
AIQLTQSPSSLSASVGDRVTITCRAS;
VL FR2:
(SEQ ID NO: 431)
LAWYQQKPGKAPKLLIY;
VL FR3:
(SEQ ID NO: 432)
SLESGVPSRFSGSGSGTDFTLTISSLQPEDFATYYC;
and
VL FR4:
(SEQ ID NO: 429)
FGGGTKVEIKRTV.

By “ABTx052 polynucleotide” is meant a nucleic acid molecule (e.g., DNA) encoding at least a fragment of an ABTx052 antibody. In an embodiment, the encoded fragment has antigen binding activity.

By “adenine” or “9H-Purin-6-amine” is meant a purine nucleobase with the molecular formula C₅H₅N₅, having the structure

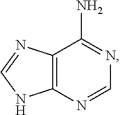

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

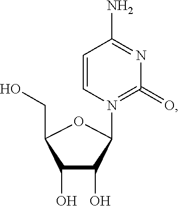

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In embodiments, the adenosine deaminase variant is selected from those described in PCT/US2020/018192, PCT/US2020/049975, PCT/US2017/045381, and PCT/US2020/028568, the full contents of which are each incorporated herein by reference in their entireties for all purposes.

By “adenosine deaminase activity” is meant catalyzing the deamination of adenine or adenosine to guanine in a polynucleotide. In some embodiments, an adenosine deaminase variant as provided herein maintains adenosine deaminase activity (e.g., at least about 30%, 40%, 50%, 60%, 70%, 80%, 90% or more of the activity of a reference adenosine deaminase (e.g., TadA*8.20 or TadA*8.19)).

By “Adenosine Base Editor (ABE)” is meant a base editor comprising an adenosine deaminase.

By “Adenosine Base Editor (ABE) polynucleotide” is meant a polynucleotide encoding an ABE. By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, such alterations are relative to the following reference sequence: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1 In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, or polypeptide, or fragments thereof.

By “alteration” is meant a change in the level, structure, or activity of an analyte, gene or polypeptide as detected by standard art known methods such as those described herein. As used herein, an alteration includes a change (e.g., increase or decrease) in expression levels. In embodiments, the increase or decrease in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant decrease, suppress, attenuate, diminish, arrest, or stabilize the development or progression of a disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)2, Fab, Fv, rlgG, and scFv fragments. Unless otherwise indicated, the term “monoclonal antibody” (mAb) is meant to include both intact molecules, as well as antibody fragments (including, for example, Fab and F(ab′)2 fragments) that are capable of specifically binding to a target protein. As used herein, the Fab and F(ab′)2 fragments refer to antibody fragments that lack the Fc fragment of an intact antibody.

Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)2 fragments, that are capable of specifically binding to a target protein. Antibodies may include chimeric antibodies; recombinant and engineered antibodies, and antigen binding fragments thereof. Exemplary functional antibody fragments comprising whole or essentially whole variable regions of both the light and heavy chains are defined as follows: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating an intact antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating an intact antibody with the enzyme pepsin, followed by reduction (two Fab′ fragments are generated per antibody molecule); and (v) F(ab′)2, a fragment of an antibody molecule containing a monovalent antigen-binding portion of an antibody molecule, obtained by treating an intact antibody with the enzyme pepsin (i.e., a dimer of Fab′ fragments held together by two disulfide bonds).

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “base editing activity” is meant acting to chemically alter a base within a polynucleotide. In one embodiment, a first base is converted to a second base. In one embodiment, the base editing activity is adenosine or adenine deaminase activity, e.g., converting A•T to G•C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is an adenine or adenosine base editor (ABE).

By “ß-globin (HBB) polypeptide” is meant a polypeptide having at least about 85% amino acid sequence identity to NCBI Accession No. NP_000509, provided below, or a fragment thereof capable of forming a dimer with a HBA1 polypeptide. In particular embodiments, a ß-globin protein comprises one or more alterations relative to the following reference sequence. In one particular embodiment, a ß-globin protein associated with sickle cell disease comprises an E6V (also termed E7V) mutation.

(SEQ ID NO: 426)
MVHLTPEEKSAVTALWGKVNVDEVGGEALGRLLVVYPWTQRFFESFGDLS
TPDAVMGNPKVKAHGKKVLGAFSDGLAHLDNLKGTFATLSELHCDKLHVD
PENFRLLGNVLVCVLAHHFGKEFTPPVQAAYQKVVAGVANALAHKYH.

By HBB polynucleotide” is meant a nucleic acid molecule that encodes an HBB polypeptide as well as the introns, exons, 3′ untranslated regions, 5′ untranslated regions, and regulatory sequences associated with its expression, or fragments thereof. In embodiments, a HBB polynucleotide is the genomic sequence, cDNA, mRNA, or gene associated with and/or required for HBB expression. Exemplary HBB polynucleotide sequences from Homo sapiens are provided below (NCBI Ref. Seq. Accessions No. NM_000518 and NG_059281).

(SEQ ID NO: 427)
ACATTTGCTTCTGACACAACTGTGTTCACTAGCAACCTCAAACAGACACC
ATGGTGCATCTGACTCCTGAGGAGAAGTCTGCCGTTACTGCCCTGTGGGG
CAAGGTGAACGTGGATGAAGTTGGTGGTGAGGCCCTGGGCAGGCTGCTGG
TGGTCTACCCTTGGACCCAGAGGTTCTTTGAGTCCTTTGGGGATCTGTCC
ACTCCTGATGCTGTTATGGGCAACCCTAAGGTGAAGGCTCATGGCAAGAA
AGTGCTCGGTGCCTTTAGTGATGGCCTGGCTCACCTGGACAACCTCAAGG
GCACCTTTGCCACACTGAGTGAGCTGCACTGTGACAAGCTGCACGTGGAT
CCTGAGAACTTCAGGCTCCTGGGCAACGTGCTGGTCTGTGTGCTGGCCCA
TCACTTTGGCAAAGAATTCACCCCACCAGTGCAGGCTGCCTATCAGAAAG
TGGTGGCTGGTGTGGCTAATGCCCTGGCCCACAAGTATCACTAAGCTCGC
TTTCTTGCTGTCCAATTTCTATTAAAGGTTCCTTTGTTCCCTAAGTCCAA
CTACTAAACTGGGGGATATTATGAAGGGCCTTGAGCATCTGGATTCTGCC
TAATAAAAAACATTTATTTTCATTGCAA.

By “cluster of differentiation 117 (CD117; C-kit; SCFR) polypeptide” is meant a polypeptide having at least about 85% amino acid sequence identity to an amino acid sequence provided at GenBank Accession No. NP_000213, which is provided below, or a fragment thereof that binds an anti-CD117 antibody. CD117 (KIT) is a type III receptor tyrosine kinase operating in cell signal transduction in several cell types. Normally KIT is activated (phosphorylated) by binding of its ligand, the stem cell factor (SCF). This leads to a phosphorylation cascade ultimately activating various transcription factors in different cell types. Such activation regulates apoptosis, cell differentiation, proliferation, chemotaxis, and cell adhesion. In some embodiments, an CD117 polypeptide or fragment thereof has SCF signaling activity.

>NP_000213.1 mast/stem cell growth factor receptor Kit isoform 1 precursor [Homo sapiens]
(SEQ ID NO: 526)
MRGARGAWDFLCVLLLLLRVQTGSSQPSVSPGEPSPPSIHPGKSDLIVRVGDEIRLLCTDPG
FVKWTFEILDETNENKQNEWITEKAEATNTGKYTCTNKHGLSNSIYVFVRDPAKLFLVDRSL
YGKEDNDTLVRCPLTDPEVTNYSLKGCQGKPLPKDLRFIPDPKAGIMIKSVKRAYHRLCLHC
SVDQEGKSVLSEKFILKVRPAFKAVPVVSVSKASYLLREGEEFTVTCTIKDVSSSVYSTWKR
ENSQTKLQEKYNSWHHGDFNYERQATLTISSARVNDSGVFMCYANNTFGSANVTTTLEVVDK
GFINIFPMINTTVFVNDGENVDLIVEYEAFPKPEHQQWIYMNRTFTDKWEDYPKSENESNIR
YVSELHLTRLKGTEGGTYTFLVSNSDVNAAIAFNVYVNTKPEILTYDRLVNGMLQCVAAGFP
EPTIDWYFCPGTEQRCSASVLPVDVQTLNSSGPPFGKLVVQSSIDSSAFKHNGTVECKAYND
VGKTSAYFNFAFKGNNKEQIHPHTLFTPLLIGFVIVAGMMCIIVMILTYKYLQKPMYEVQWK
VVEEINGNNYVYIDPTQLPYDHKWEFPRNRLSFGKTLGAGAFGKVVEATAYGLIKSDAAMTV
AVKMLKPSAHLTEREALMSELKVLSYLGNHMNIVNLLGACTIGGPTLVITEYCCYGDLLNFL
RRKRDSFICSKQEDHAEAALYKNLLHSKESSCSDSTNEYMDMKPGVSYVVPTKADKRRSVRI
GSYIERDVTPAIMEDDELALDLEDLLSFSYQVAKGMAFLASKNCIHRDLAARNILLTHGRIT
KICDFGLARDIKNDSNYVVKGNARLPVKWMAPESIFNCVYTFESDVWSYGIFLWELFSLGSS
PYPGMPVDSKFYKMIKEGFRMLSPEHAPAEMYDIMKTCWDADPLKRPTFKQIVQLIEKQISE
STNHIYSNLANCSPNRQKPVVDHSVRINSVGSTASSSQPLLVHDDV
>CD117 variant with S261G alteration and N260D alteration (shown in bold-underline)
(SEQ ID NO: 527)
MRGARGAWDFLCVLLLLLRVQTGSSQPSVSPGEPSPPSIHPGKSDLIVRVGDEIRLLCTDPG
FVKWTFEILDETNENKQNEWITEKAEATNTGKYTCTNKHGLSNSIYVFVRDPAKLFLVDRSL
YGKEDNDTLVRCPLTDPEVTNYSLKGCQGKPLPKDLRFIPDPKAGIMIKSVKRAYHRLCLHC
SVDQEGKSVLSEKFILKVRPAFKAVPVVSVSKASYLLREGEEFTVTCTIKDVSSSVYSTWKR
ENSQTKLQEKY [symbol] WHHGDFNYERQATLTISSARVNDSGVFMCYANNTFGSANVTTTLEVVDK
GFINIFPMINTTVFVNDGENVDLIVEYEAFPKPEHQQWIYMNRTFTDKWEDYPKSENESNIR
YVSELHLTRLKGTEGGTYTFLVSNSDVNAAIAFNVYVNTKPEILTYDRLVNGMLQCVAAGFP
EPTIDWYFCPGTEQRCSASVLPVDVQTLNSSGPPFGKLVVQSSIDSSAFKHNGTVECKAYND
1
VGKTSAYFNFAFKGNNKEQIHPHTLFTPLLIGFVIVAGMMCIIVMILTYKYLQKPMYEVQWK
VVEEINGNNYVYIDPTQLPYDHKWEFPRNRLSFGKTLGAGAFGKVVEATAYGLIKSDAAMTV
AVKMLKPSAHLTEREALMSELKVLSYLGNHMNIVNLLGACTIGGPTLVITEYCCYGDLLNFL
RRKRDSFICSKQEDHAEAALYKNLLHSKESSCSDSTNEYMDMKPGVSYVVPTKADKRRSVRI
GSYIERDVTPAIMEDDELALDLEDLLSFSYQVAKGMAFLASKNCIHRDLAARNILLTHGRIT
KICDFGLARDIKNDSNYVVKGNARLPVKWMAPESIFNCVYTFESDVWSYGIFLWELFSLGSS
PYPGMPVDSKFYKMIKEGFRMLSPEHAPAEMYDIMKTCWDADPLKRPTFKQIVQLIEKQISE
STNHIYSNLANCSPNRQKPVVDHSVRINSVGSTASSSQPLLVHDDV
>CD117 variant with S261G alteration (shown in bold-underline)
(SEQ ID NO: 528)
MRGARGAWDFLCVLLLLLRVQTGSSQPSVSPGEPSPPSIHPGKSDLIVRVGDEIRLLCTDPG
FVKWTFEILDETNENKQNEWITEKAEATNTGKYTCTNKHGLSNSIYVFVRDPAKLFLVDRSL
YGKEDNDTLVRCPLTDPEVTNYSLKGCQGKPLPKDLRFIPDPKAGIMIKSVKRAYHRLCLHC
SVDQEGKSVLSEKFILKVRPAFKAVPVVSVSKASYLLREGEEFTVTCTIKDVSSSVYSTWKR
ENSQTKLQEKYNGWHHGDFNYERQATLTISSARVNDSGVFMCYANNTFGSANVTTTLEVVDK
GFINIFPMINTTVFVNDGENVDLIVEYEAFPKPEHQQWIYMNRTFTDKWEDYPKSENESNIR
YVSELHLTRLKGTEGGTYTFLVSNSDVNAAIAFNVYVNTKPEILTYDRLVNGMLQCVAAGFP
EPTIDWYFCPGTEQRCSASVLPVDVQTLNSSGPPFGKLVVQSSIDSSAFKHNGTVECKAYND
VGKTSAYFNFAFKGNNKEQIHPHTLFTPLLIGFVIVAGMMCIIVMILTYKYLQKPMYEVQWK
VVEEINGNNYVYIDPTQLPYDHKWEFPRNRLSFGKTLGAGAFGKVVEATAYGLIKSDAAMTV
AVKMLKPSAHLTEREALMSELKVLSYLGNHMNIVNLLGACTIGGPTLVITEYCCYGDLLNFL
RRKRDSFICSKQEDHAEAALYKNLLHSKESSCSDSTNEYMDMKPGVSYVVPTKADKRRSVRI
GSYIERDVTPAIMEDDELALDLEDLLSFSYQVAKGMAFLASKNCIHRDLAARNILLTHGRIT
KICDFGLARDIKNDSNYVVKGNARLPVKWMAPESIFNCVYTFESDVWSYGIFLWELFSLGSS
PYPGMPVDSKFYKMIKEGFRMLSPEHAPAEMYDIMKTCWDADPLKRPTFKQIVQLIEKQISE
STNHIYSNLANCSPNRQKPVVDHSVRINSVGSTASSSQPLLVHDDV
>CD117 variant with Y259C and N260D alterations (shown in bold-underline)
(SEQ ID NO: 529)
MRGARGAWDFLCVLLLLLRVQTGSSQPSVSPGEPSPPSIHPGKSDLIVRVGDEIRLLCTDPG
FVKWTFEILDETNENKQNEWITEKAEATNTGKYTCTNKHGLSNSIYVFVRDPAKLFLVDRSL
YGKEDNDTLVRCPLTDPEVTNYSLKGCQGKPLPKDLRFIPDPKAGIMIKSVKRAYHRLCLHC
SVDQEGKSVLSEKFILKVRPAFKAVPVVSVSKASYLLREGEEFTVTCTIKDVSSSVYSTWKR
ENSQTKLQEK [symbol] SWHHGDFNYERQATLTISSARVNDSGVFMCYANNTFGSANVTTTLEVVDK
GFINIFPMINTTVFVNDGENVDLIVEYEAFPKPEHQQWIYMNRTFTDKWEDYPKSENESNIR
YVSELHLTRLKGTEGGTYTFLVSNSDVNAAIAFNVYVNTKPEILTYDRLVNGMLQCVAAGFP
EPTIDWYFCPGTEQRCSASVLPVDVQTLNSSGPPFGKLVVQSSIDSSAFKHNGTVECKAYND
VGKTSAYFNFAFKGNNKEQIHPHTLFTPLLIGFVIVAGMMCIIVMILTYKYLQKPMYEVQWK
VVEEINGNNYVYIDPTQLPYDHKWEFPRNRLSFGKTLGAGAFGKVVEATAYGLIKSDAAMTV
AVKMLKPSAHLTEREALMSELKVLSYLGNHMNIVNLLGACTIGGPTLVITEYCCYGDLLNFL
RRKRDSFICSKQEDHAEAALYKNLLHSKESSCSDSTNEYMDMKPGVSYVVPTKADKRRSVRI
GSYIERDVTPAIMEDDELALDLEDLLSFSYQVAKGMAFLASKNCIHRDLAARNILLTHGRIT
KICDFGLARDIKNDSNYVVKGNARLPVKWMAPESIFNCVYTFESDVWSYGIFLWELFSLGSS
PYPGMPVDSKFYKMIKEGFRMLSPEHAPAEMYDIMKTCWDADPLKRPTFKQIVQLIEKQISE
STNHIYSNLANCSPNRQKPVVDHSVRINSVGSTASSSQPLLVHDDV
>CD117 variant with an N260D alteration (shown in bold-underline)
(SEQ ID NO: 530)
MRGARGAWDFLCVLLLLLRVQTGSSQPSVSPGEPSPPSIHPGKSDLIVRVGDEIRLLCTDPG
FVKWTFEILDETNENKQNEWITEKAEATNTGKYTCTNKHGLSNSIYVFVRDPAKLFLVDRSL
YGKEDNDTLVRCPLTDPEVTNYSLKGCQGKPLPKDLRFIPDPKAGIMIKSVKRAYHRLCLHC
SVDQEGKSVLSEKFILKVRPAFKAVPVVSVSKASYLLREGEEFTVTCTIKDVSSSVYSTWKR
ENSQTKLQEKY [symbol] SWHHGDFNYERQATLTISSARVNDSGVFMCYANNTFGSANVTTTLEVVDK
GFINIFPMINTTVFVNDGENVDLIVEYEAFPKPEHQQWIYMNRTFTDKWEDYPKSENESNIR
YVSELHLTRLKGTEGGTYTFLVSNSDVNAAIAFNVYVNTKPEILTYDRLVNGMLQCVAAGFP
EPTIDWYFCPGTEQRCSASVLPVDVQTLNSSGPPFGKLVVQSSIDSSAFKHNGTVECKAYND
VGKTSAYFNFAFKGNNKEQIHPHTLFTPLLIGFVIVAGMMCIIVMILTYKYLQKPMYEVQWK
VVEEINGNNYVYIDPTQLPYDHKWEFPRNRLSFGKTLGAGAFGKVVEATAYGLIKSDAAMTV
AVKMLKPSAHLTEREALMSELKVLSYLGNHMNIVNLLGACTIGGPTLVITEYCCYGDLLNFL
RRKRDSFICSKQEDHAEAALYKNLLHSKESSCSDSTNEYMDMKPGVSYVVPTKADKRRSVRI
GSYIERDVTPAIMEDDELALDLEDLLSFSYQVAKGMAFLASKNCIHRDLAARNILLTHGRIT
KICDFGLARDIKNDSNYVVKGNARLPVKWMAPESIFNCVYTFESDVWSYGIFLWELFSLGSS
PYPGMPVDSKFYKMIKEGFRMLSPEHAPAEMYDIMKTCWDADPLKRPTFKQIVQLIEKQISE
STNHIYSNLANCSPNRQKPVVDHSVRINSVGSTASSSQPLLVHDDV
>CD117 variant with an S251G alteration (shown in bold-underline)
(SEQ ID NO: 531)
MRGARGAWDFLCVLLLLLRVQTGSSQPSVSPGEPSPPSIHPGKSDLIVRVGDEIRLLCTDPG
FVKWTFEILDETNENKQNEWITEKAEATNTGKYTCTNKHGLSNSIYVFVRDPAKLFLVDRSL
YGKEDNDTLVRCPLTDPEVTNYSLKGCQGKPLPKDLRFIPDPKAGIMIKSVKRAYHRLCLHC
SVDQEGKSVLSEKFILKVRPAFKAVPVVSVSKASYLLREGEEFTVTCTIKDVSSSVYSTWKR
EN [symbol] QTKLQEKYNSWHHGDFNYERQATLTISSARVNDSGVFMCYANNTFGSANVTTTLEVVDK
GFINIFPMINTTVFVNDGENVDLIVEYEAFPKPEHQQWIYMNRTFTDKWEDYPKSENESNIR
YVSELHLTRLKGTEGGTYTFLVSNSDVNAAIAFNVYVNTKPEILTYDRLVNGMLQCVAAGFP
EPTIDWYFCPGTEQRCSASVLPVDVQTLNSSGPPFGKLVVQSSIDSSAFKHNGTVECKAYND
VGKTSAYFNFAFKGNNKEQIHPHTLFTPLLIGFVIVAGMMCIIVMILTYKYLQKPMYEVQWK
VVEEINGNNYVYIDPTQLPYDHKWEFPRNRLSFGKTLGAGAFGKVVEATAYGLIKSDAAMTV
AVKMLKPSAHLTEREALMSELKVLSYLGNHMNIVNLLGACTIGGPTLVITEYCCYGDLLNFL
RRKRDSFICSKQEDHAEAALYKNLLHSKESSCSDSTNEYMDMKPGVSYVVPTKADKRRSVRI
GSYIERDVTPAIMEDDELALDLEDLLSFSYQVAKGMAFLASKNCIHRDLAARNILLTHGRIT
KICDFGLARDIKNDSNYVVKGNARLPVKWMAPESIFNCVYTFESDVWSYGIFLWELFSLGSS
PYPGMPVDSKFYKMIKEGFRMLSPEHAPAEMYDIMKTCWDADPLKRPTFKQIVQLIEKQISE
STNHIYSNLANCSPNRQKPVVDHSVRINSVGSTASSSQPLLVHIDDV

By “cluster of differentiation 117 (CD117; C-kit; SCFR) polynucleotide” is meant a nucleic acid molecule that encodes a CD117 polypeptide as well as the introns, exons, 3′ untranslated regions, 5′ untranslated regions, and regulatory sequences associated with its expression, or fragments thereof. In embodiments, a CD117 polynucleotide is the genomic sequence, cDNA, mRNA, or gene associated with and/or required for CD117 expression. An exemplary CD117 polynucleotide sequence from Homo sapiens is provided below (NCBI Ref. Seq. Accession No. NM_000222.2), and an exemplary CD117 gene sequence is provided at ENSEMBL Accession No. ENSG00000157404.

>NM 000222.2 Homo sapiens KIT proto-oncogene, receptor tyrosine kinase (KIT),
transcript variant 1, mRNA
(SEQ ID NO: 532)
TCTGGGGGCTCGGCTTTGCCGCGCTCGCTGCACTTGGGCGAGAGCTGGAACGTGGACCAGAG
CTCGGATCCCATCGCAGCTACCGCGATGAGAGGCGCTCGCGGCGCCTGGGATTTTCTCTGCG
TTCTGCTCCTACTGCTTCGCGTCCAGACAGGCTCTTCTCAACCATCTGTGAGTCCAGGGGAA
CCGTCTCCACCATCCATCCATCCAGGAAAATCAGACTTAATAGTCCGCGTGGGCGACGAGAT
TAGGCTGTTATGCACTGATCCGGGCTTTGTCAAATGGACTTTTGAGATCCTGGATGAAACGA
ATGAGAATAAGCAGAATGAATGGATCACGGAAAAGGCAGAAGCCACCAACACCGGCAAATAC
ACGTGCACCAACAAACACGGCTTAAGCAATTCCATTTATGTGTTTGTTAGAGATCCTGCCAA
GCTTTTCCTTGTTGACCGCTCCTTGTATGGGAAAGAAGACAACGACACGCTGGTCCGCTGTC
CTCTCACAGACCCAGAAGTGACCAATTATTCCCTCAAGGGGTGCCAGGGGAAGCCTCTTCCC
AAGGACTTGAGGTTTATTCCTGACCCCAAGGCGGGCATCATGATCAAAAGTGTGAAACGCGC
CTACCATCGGCTCTGTCTGCATTGTTCTGTGGACCAGGAGGGCAAGTCAGTGCTGTCGGAAA
AATTCATCCTGAAAGTGAGGCCAGCCTTCAAAGCTGTGCCTGTTGTGTCTGTGTCCAAAGCA
AGCTATCTTCTTAGGGAAGGGGAAGAATTCACAGTGACGTGCACAATAAAAGATGTGTCTAG
TTCTGTGTACTCAACGTGGAAAAGAGAAAACAGTCAGACTAAACTACAGGAGAAATATAATA
GCTGGCATCACGGTGACTTCAATTATGAACGTCAGGCAACGTTGACTATCAGTTCAGCGAGA
GTTAATGATTCTGGAGTGTTCATGTGTTATGCCAATAATACTTTTGGATCAGCAAATGTCAC
AACAACCTTGGAAGTAGTAGATAAAGGATTCATTAATATCTTCCCCATGATAAACACTACAG
TATTTGTAAACGATGGAGAAAATGTAGATTTGATTGTTGAATATGAAGCATTCCCCAAACCT
GAACACCAGCAGTGGATCTATATGAACAGAACCTTCACTGATAAATGGGAAGATTATCCCAA
GTCTGAGAATGAAAGTAATATCAGATACGTAAGTGAACTTCATCTAACGAGATTAAAAGGCA
CCGAAGGAGGCACTTACACATTCCTAGTGTCCAATTCTGACGTCAATGCTGCCATAGCATTT
AATGTTTATGTGAATACAAAACCAGAAATCCTGACTTACGACAGGCTCGTGAATGGCATGCT
CCAATGTGTGGCAGCAGGATTCCCAGAGCCCACAATAGATTGGTATTTTTGTCCAGGAACTG
AGCAGAGATGCTCTGCTTCTGTACTGCCAGTGGATGTGCAGACACTAAACTCATCTGGGCCA
CCGTTTGGAAAGCTAGTGGTTCAGAGTTCTATAGATTCTAGTGCATTCAAGCACAATGGCAC
GGTTGAATGTAAGGCTTACAACGATGTGGGCAAGACTTCTGCCTATTTTAACTTTGCATTTA
AAGGTAACAACAAAGAGCAAATCCATCCCCACACCCTGTTCACTCCTTTGCTGATTGGTTTC
GTAATCGTAGCTGGCATGATGTGCATTATTGTGATGATTCTGACCTACAAATATTTACAGAA
ACCCATGTATGAAGTACAGTGGAAGGTTGTTGAGGAGATAAATGGAAACAATTATGTTTACA
TAGACCCAACACAACTTCCTTATGATCACAAATGGGAGTTTCCCAGAAACAGGCTGAGTTTT
GGGAAAACCCTGGGTGCTGGAGCTTTCGGGAAGGTTGTTGAGGCAACTGCTTATGGCTTAAT
TAAGTCAGATGCGGCCATGACTGTCGCTGTAAAGATGCTCAAGCCGAGTGCCCATTTGACAG
AACGGGAAGCCCTCATGTCTGAACTCAAAGTCCTGAGTTACCTTGGTAATCACATGAATATT
GTGAATCTACTTGGAGCCTGCACCATTGGAGGGCCCACCCTGGTCATTACAGAATATTGTTG
CTATGGTGATCTTTTGAATTTTTTGAGAAGAAAACGTGATTCATTTATTTGTTCAAAGCAGG
AAGATCATGCAGAAGCTGCACTTTATAAGAATCTTCTGCATTCAAAGGAGTCTTCCTGCAGC
GATAGTACTAATGAGTACATGGACATGAAACCTGGAGTTTCTTATGTTGTCCCAACCAAGGC
CGACAAAAGGAGATCTGTGAGAATAGGCTCATACATAGAAAGAGATGTGACTCCCGCCATCA
TGGAGGATGACGAGTTGGCCCTAGACTTAGAAGACTTGCTGAGCTTTTCTTACCAGGTGGCA
AAGGGCATGGCTTTCCTCGCCTCCAAGAATTGTATTCACAGAGACTTGGCAGCCAGAAATAT
CCTCCTTACTCATGGTCGGATCACAAAGATTTGTGATTTTGGTCTAGCCAGAGACATCAAGA
ATGATTCTAATTATGTGGTTAAAGGAAACGCTCGACTACCTGTGAAGTGGATGGCACCTGAA
AGCATTTTCAACTGTGTATACACGTTTGAAAGTGACGTCTGGTCCTATGGGATTTTTCTTTG
GGAGCTGTTCTCTTTAGGAAGCAGCCCCTATCCTGGAATGCCGGTCGATTCTAAGTTCTACA
AGATGATCAAGGAAGGCTTCCGGATGCTCAGCCCTGAACACGCACCTGCTGAAATGTATGAC
ATAATGAAGACTTGCTGGGATGCAGATCCCCTAAAAAGACCAACATTCAAGCAAATTGTTCA
GCTAATTGAGAAGCAGATTTCAGAGAGCACCAATCATATTTACTCCAACTTAGCAAACTGCA
GCCCCAACCGACAGAAGCCCGTGGTAGACCATTCTGTGCGGATCAATTCTGTCGGCAGCACC
GCTTCCTCCTCCCAGCCTCTGCTTGTGCACGACGATGTCTGAGCAGAATCAGTGTTTGGGTC
ACCCCTCCAGGAATGATCTCTTCTTTTGGCTTCCATGATGGTTATTTTCTTTTCTTTCAACT
TGCATCCAACTCCAGGATAGTGGGCACCCCACTGCAATCCTGTCTTTCTGAGCACACTTTAG
TGGCCGATGATTTTTGTCATCAGCCACCATCCTATTGCAAAGGTTCCAACTGTATATATTCC
CAATAGCAACGTAGCTTCTACCATGAACAGAAAACATTCTGATTTGGAAAAAGAGAGGGAGG
TATGGACTGGGGGCCAGAGTCCTTTCCAAGGCTTCTCCAATTCTGCCCAAAAATATGGTTGA
TAGTTTACCTGAATAAATGGTAGTAATCACAGTTGGCCTTCAGAACCATCCATAGTAGTATG
ATGATACAAGATTAGAAGCTGAAAACCTAAGTCCTTTATGTGGAAAACAGAACATCATTAGA
ACAAAGGACAGAGTATGAACACCTGGGCTTAAGAAATCTAGTATTTCATGCTGGGAATGAGA
CATAGGCCATGAAAAAAATGATCCCCAAGTGTGAACAAAAGATGCTCTTCTGTGGACCACTG
CATGAGCTTTTATACTACCGACCTGGTTTTTAAATAGAGTTTGCTATTAGAGCATTGAATTG
GAGAGAAGGCCTCCCTAGCCAGCACTTGTATATACGCATCTATAAATTGTCCGTGTTCATAC
ATTTGAGGGGAAAACACCATAAGGTTTCGTTTCTGTATACAACCCTGGCATTATGTCCACTG
TGTATAGAAGTAGATTAAGAGCCATATAAGTTTGAAGGAAACAGTTAATACCATTTTTTAAG
GAAACAATATAACCACAAAGCACAGTTTGAACAAAATCTCCTCTTTTAGCTGATGAACTTAT
TCTGTAGATTCTGTGGAACAAGCCTATCAGCTTCAGAATGGCATTGTACTCAATGGATTTGA
TGCTGTTTGACAAAGTTACTGATTCACTGCATGGCTCCCACAGGAGTGGGAAAACACTGCCA
TCTTAGTTTGGATTCTTATGTAGCAGGAAATAAAGTATAGGTTTAGCCTCCTTCGCAGGCAT
GTCCTGGACACCGGGCCAGTATCTATATATGTGTATGTACGTTTGTATGTGTGTAGACAAAT
ATTTGGAGGGGTATTTTTGCCCTGAGTCCAAGAGGGTCCTTTAGTACCTGAAAAGTAACTTG
GCTTTCATTATTAGTACTGCTCTTGTTTCTTTTCACATAGCTGTCTAGAGTAGCTTACCAGA
AGCTTCCATAGTGGTGCAGAGGAAGTGGAAGGCATCAGTCCCTATGTATTTGCAGTTCACCT
GCACTTAAGGCACTCTGTTATTTAGACTCATCTTACTGTACCTGTTCCTTAGACCTTCCATA
ATGCTACTGTCTCACTGAAACATTTAAATTTTACCCTTTAGACTGTAGCCTGGATATTATTC
TTGTAGTTTACCTCTTTAAAAACAAAACAAAACAAAACAAAAAACTCCCCTTCCTCACTGCC
CAATATAAAAGGCAAATGTGTACATGGCAGAGTTTGTGTGTTGTCTTGAAAGATTCAGGTAT
GTTGCCTTTATGGTTTCCCCCTTCTACATTTCTTAGACTACATTTAGAGAACTGTGGCCGTT
ATCTGGAAGTAACCATTTGCACTGGAGTTCTATGCTCTCGCACCTTTCCAAAGTTAACAGAT
TTTGGGGTTGTGTTGTCACCCAAGAGATTGTTGTTTGCCATACTTTGTCTGAAAAATTCCTT
TGTGTTTCTATTGACTTCAATGATAGTAAGAAAAGTGGTTGTTAGTTATAGATGTCTAGGTA
CTTCAGGGGCACTTCATTGAGAGTTTTGTCTTGGATATTCTTGAAAGTTTATATTTTTATAA
TTTTTTCTTACATCAGATGTTTCTTTGCAGTGGCTTAATGTTTGAAATTATTTTGTGGCTTT
TTTTGTAAATATTGAAATGTAGCAATAATGTCTTTTGAATATTCCCAAGCCCATGAGTCCTT
GAAAATATTTTTTATATATACAGTAACTTTATGTGTAAATACATAAGCGGCGTAAGTTTAAA
GGATGTTGGTGTTCCACGTGTTTTATTCCTGTATGTTGTCCAATTGTTGACAGTTCTGAAGA
ATTCTAATAAAATGTACATATATAAATCAAAAAAAAAAAAAAAA
>NG_059281.1: 5001-6608 Homo sapiens hemoglobin subunit beta (HBB), RefSeqGene
(LRG_1232) on chromosome 11; an A altered to T in Sickle cell disease is indicated in bold;
The bold-underlined T indicates a SNP_that is a C in some sickle cell patients. The underlined
ATG is the start codon.
(SEQ ID NO: 428)
ACATTTGCTTCTGACACAACTGTGTTCACTAGCAACCTCAAACAGACACCATGGTGCATCTG
ACTCCTGAGGAGAAGTCTGCCGTTACTGCCCTGTGGGGCAAGGTGAACGTGGATGAAGTTGG
TGGTGAGGCCCTGGGCAGGTTGGTATCAAGGTTACAAGACAGGTTTAAGGAGACCAATAGAA
ACTGGGCATGTGGAGACAGAGAAGACTCTTGGGTTTCTGATAGGCACTGACTCTCTCTGCCT
ATTGGTCTATTTTCCCACCCTTAGGCTGCTGGTGGTCTACCCTTGGACCCAGAGGTTCTTTG
AGTCCTTTGGGGATCTGTCCACTCCTGATGCTGTTATGGGCAACCCTAAGGTGAAGGCTCAT
GGCAAGAAAGTGCTCGGTGCCTTTAGTGATGGCCTGGCTCACCTGGACAACCTCAAGGGCAC
CTTTGCCACACTGAGTGAGCTGCACTGTGACAAGCTGCACGTGGATCCTGAGAACTTCAGGG
TGAGTCTATGGGACGCTTGATGTTTTCTTTCCCCTTCTTTTCTATGGTTAAGTTCATGTCAT
AGGAAGGGGATAAGTAACAGGGTACAGTTTAGAATGGGAAACAGACGAATGATTGCATCAGT
GTGGAAGTCTCAGGATCGTTTTAGTTTCTTTTATTTGCTGTTCATAACAATTGTTTTCTTTT
GTTTAATTCTTGCTTTCTTTTTTTTTCTTCTCCGCAATTTTTACTATTATACTTAATGCCTT
AACATTGTGTATAACAAAAGGAAATATCTCTGAGATACATTAAGTAACTTAAAAAAAAACTT
TACACAGTCTGCCTAGTACATTACTATTTGGAATATATGTGTGCTTATTTGCATATTCATAA
TCTCCCTACTTTATTTTCTTTTATTTTTAATTGATACATAATCATTATACATATTTATGGGT
TAAAGTGTAATGTTTTAATATGTGTACACATATTGACCAAATCAGGGTAATTTTGCATTTGT
AATTTTAAAAAATGCTTTCTTCTTTTAATATACTTTTTTGTTTATCTTATTTCTAATACTTT
CCCTAATCTCTTTCTTTCAGGGCAATAATGATACAATGTATCATGCCTCTTTGCACCATTCT
AAAGAATAACAGTGATAATTTCTGGGTTAAGGCAATAGCAATATCTCTGCATATAAATATTT
CTGCATATAAATTGTAACTGATGTAAGAGGTTTCATATTGCTAATAGCAGCTACAATCCAGC
TACCATTCTGCTTTTATTTTATGGTTGGGATAAGGCTGGATTATTCTGAGTCCAAGCTAGGC
CCTTTTGCTAATCATGTTCATACCTCTTATCTTCCTCCCACAGCTCCTGGGCAACGTGCTGG
TCTGTGTGCTGGCCCATCACTTTGGCAAAGAATTCACCCCACCAGTGCAGGCTGCCTATCAG
AAAGTGGTGGCTGGTGTGGCTAATGCCCTGGCCCACAAGTATCACTAAGCTCGCTTTCTTGC
TGTCCAATTTCTATTAAAGGTTCCTTTGTTCCCTAAGTCCAACTACTAAACTGGGGGATATT
ATGAAGGGCCTTGAGCATCTGGATTCTGCCTAATAAAAAACATTTATTTTCATTGCAA.

By “hemoglobin, gamma A (HBG1) polypeptide” is meant a polypeptide having at least about 85% amino acid sequence identity to Genbank Accession No. CAA23771.1, provided below, or a fragment thereof capable of forming a protein complex with alpha hemoglobin subunits.

(SEQ ID NO: 429)
MGHFTEEDKATITSLWGKVNVEDAGGETLGRLLVVYPWTQRFFDSFGNL
SSASAIMGNPKVKAHGKKVLTSLGDAIKHLDDLKGTFAQLSELHCDKLH
VDPENFKLLGNVLVTVLAIHFGKEFTPEVQASWQKMVTAVASALSSRYH.

By HBG1 polynucleotide” is meant a nucleic acid molecule that encodes an HBG1 polypeptide as well as the introns, exons, 3′ untranslated regions, 5′ untranslated regions, and regulatory sequences associated with its expression, or fragments thereof. In embodiments, a HBG1 polynucleotide is the genomic sequence, cDNA, mRNA, or gene associated with and/or required for HBG1 expression. An exemplary HBB polynucleotide sequence from Homo sapiens is provided on ENSEMBL at accession no. GRCh38:11:5248044:5259425:1, the reverse-complement of which is provided below (SEQ ID NO: 430). In the below sequence, exons encoding HBG1 are shown in bold and an exemplary HBG1 promoter region corresponds to a region 5′ of the first exon encoding HBG1 (e.g., the first 100, 200, 300, or 400 nucleotides 5′ of the first exon), or portions thereof.

(SEQ ID NO: 430)
CCTTCTCACTTGGGTAGGCTCTGTCAGAGGGAAAGTCTAGGCCTCAAGGCTGAGACTTTTGT
CCCATGAGGTGTTCCCTTGATGTAGCACAGTCCCCCTTTTCCTAGGCGTGGGGCTTCCTGAG
AGCCGAACTGTAGTGATTGTTATCTCTCTTCTGGATCTAGCCACCCATCAGGTCTACCAGAC
TCCAGGCTGGTACTGGGGTTTGTCTGCACAGAGTCTTGTGACGTGAACCATCTGTGGGTCTC
TCAGCCATAGATACAACCACCTGCTCCAATGGAGGTGGCAGAGGATGAAATGGACTCTGTGA
GGGTCCTTACTTTTGGTTGTTCAATGCACTATTTTTGTGCTGGTTGGCCTCCTGCCAGGAGG
TGGCACTTTCTAGAAAGCATCAGCAGAGGCAGTCAGGTGGTGGTGGCTGGGGGGGCTGGGGC
ACCCTAGAACTCCCAAGAATATATGCCCTTTGTCTTCAGCTACCAGGGTGAGTAAGGAAGGA
CCATCAGGTGGGGGCAGGACTAGTCGTGTCTGAGCTCAGAGTCTCCTTGGGCAGGTCTTTCT
GTGGCTACTGTGGGAGGATGGGGGTGTAGTTTCCAGGTCAATGGATTTATGTTCCTAGGACA
ATTATGGCTGCCTCTGCTGTGTCATGCAGGTCATCAGGAAAGTGGGGGAAAGCAAGCAGTCA
CGTGACTTGCCCAGCTCCCATGCAACTCAAAAGGTTGGTCTCACTTCCAGCGTGCACCCTCC
CCCGCAACAGCACCGAATCTGTTTCCATGCAGTCAGTGAGCAAGGCTGAGAACTTGCCCCAG
GCTACCAGCTGCGAAACCAAGTAGGGCTGTCCTACTTCCCTGCCAGTGGAGTCTGCACACCA
AATTCATGTCCCCCCACCAACCCCCCCACTGCCCAGCCCCTAGATCTGGCCAGGTGGAGATT
TTCTTTTTCCTGTCATCTTTTCCCAGTTCCTCTGGCAGCCCTCCCAAATGACCCCTGTGAGG
CAAGGCAGAAATGGCTTCCTAGGGGACCCAGAGAGCCCACAGGGCTTTTCCCGCTGCTTCCT
CTACCCCTGTATTTTGCTTGGCCCTCTAAATTGACTCAGCTCCAGGTAAGGTCAGAATCTTC
TCCTGTGGTCTAGATCTTCAGGTTCCCCAGTGAGGATGTGTGTTTGGGGGTAGACGGTCCCC
CTTTTCCACTTCCACAGTTTGGGCACTCACAATATTTGGGGTGTTTCCCGGGTCCTGCAGGA
GCAATCTGCTTCTTTCAGAGGGTGTGTGCGTTCTCTCAGCTTTCTTGATTTATTTCTGCAGG
TGGTTCTGCAAAAAAAATTCCTGATGGGAGACTTCACATGCTGCTCTGTGCATCCGAGTGGG
AGCTGCAATGTACTTCTGCTGCCTCCCATCTGCCATCACCCTCTAATTTGTCGGTAATATGC
ATTTTTAATCAATCTTTTTTTCTCTCTCTCTCTTTTTCTTCTCCCCCAAAACTATACTGCCC
TTTGATATCAAGGAATCAAGGACGTGATGTTGAGGGGTGGGCAGTGGATACACTCTTTACCC
CTTAGGGAGCTATATCTAGATTTAGATATTGCCAATTCAAGATAACTTAATTGAAAGCAAAT
TCATAATGAATACACACACACACACACACATCTGCATGACAAGATTTTTAATAGTTGAAAGA
ATAACTAATAATTGTCCACAGGCAATAAGGGCTTTTTAAGCAAAACAGTTGTGATAAACAGG
TCATTCTTAGAATAGTAATCCAGCCAATAGTACAGGTTGCTTAGAGATTATGTCATTACCAG
AGTTAAAATTCTATAATGGCTTCTCACTCCCTACCACTGAGGACAAGTTTATGTCCTTAGGT
TTATGCTTCCCTGAAACAATACCACCTGCTATTCTCCACTTTACATATCAACGGCACTGGTT
CTTTATCTAACTCTCTGGCACAGCAGGAGTTTGTTTTCTTCTGCTTCAGAGCTTTGAATTTA
CTATTTCAGCTTCTAAACTTTATTTGGCAATGCCTTCCCATGGCAGATTCCTTCTGTCATTT
TGCCTCTGTTCGAATACTTTCTCCTTAATTTCATTCTTAGTTAATAATATCTGAAATTATTT
TGTTGTTTAACTTAATTATTAATTTTATGTATGTTCTACCTAGATTATAATCTTCAGAGGAA
AGTTTTATTCTCTGACTTATTTAACTTAAATGCCCACTACTTTAAAAATTATGACATTTATT
TAACAGATATTTGCTGAACAAATGTTTGAAAATACATGGGAAAGAATGCTTGAAAACACTTG
AAATTGCTTGTGTAAAGAAACAGTTTTATCAGTTAGGATTTAATCAATGTCAGAAGCAATGA
TATAGGAAAAATCGAGGAATAAGACAGTTATGGATAAGGAGAAATCAACAAACTCTTAAAAG
ATATTGCCTCAAAAGCATAAGAGGAAATAAGGGTTTATACATGACTTTTAGAACACTGCCTT
GGTTTTTGGATAAATGGGGAAGTTGTTTGAAAACAGGAGGGATCCTAGATATTCCTTAGTCT
GAGGAGGAGCAATTAAGATTCACTTGTTTAGAGGCTGGGAGTGGTGGCTCACGCCTGTAATC
CCAGAATTTTGGGAGGCCAAGGCAGGCAGATCACCTGAGGTCAAGAGTTCAAGACCAACCTG
GCCAACATGGTGAAATCCCATCTCTACAAAAATACAAAAATTAGACAGGCATGATGGCAAGT
GCCTGTAATCCCAGCTACTTGGGAGGCTGAGGAAGGAGAATTGCTTGAACCTGGAAGGCAGG
AGTTGCAGTGAGCCGAGATCATACCACTGCACTCCAGCCTGGGTGACAGAACAAGACTCTGT
CTCAAAAAAAAAAAAGAGAGATTCAAAAGATTCACTTGTTTAGGCCTTAGCGGGCTTAGACA
CCAGTCTCTGACACATTCTTAAAGGTCAGGCTCTACAAATGGAACCCAACCAGACTCTCAGA
TATGGCCAAAGATCTATACACACCCATCTCACAGATCCCCTATCTTAAAGAGACCCTAATTT
GGGTTCACCTCAGTCTCTATAATCTGTACCAGCATACCAATAAAAATCTTTCTCACCCATCC
TTAGATTGAGAGAAGTCACTTATTATTATGTGAGTAACTGGAAGATACTGATAAGTTGACAA
ATCTTTTTCTTTCCTTTCTTATTCAACTTTTATTTTAACTTCCAAAGAACAAGTGCAATATG
TGCAGCTTTGTTGCGCAGGTCAACATGTATCTTTCTGGTCTTTTAGCCGCCTAACACTTTGA
GCAGATATAAGCCTTACACAGGATTATGAAGTCTGAAAGGATTCCACCAATATTATTATAAT
TCCTATCAACCTGATAGGTTAGGGGAAGGTAGAGCTCTCCTCCAATAAGCCAGATTTCCAGA
GTTTCTGACGTCATAATCTACCAAGGTCATGGATCGAGTTCAGAGAAAAAACAAAAGCAAAA
CCAAACCTACCAAAAAATAAAAATCCCAAAGAAAAAATAAAGAAAAAAACAGCATGAATACT
TCCTGCCATGTTAAGTGGCCAATATGTCAGAAACAGCACTGAGTTACAGATAAAGATGTCTA
AACTACAGTGACATCCCAGCTGTCACAGTGTGTGGACTATTAGTCAATAAAACAGTCCCTGC
CTCTTAAGAGTTGTTTTCCATGCAAATACATGTCTTATGTCTTAGAATAAGATTCCCTAAGA
AGTGAACCTAGCATTTATACAAGATAATTAATTCTAATCCATAGTATCTGGTAAAGAGCATT
CTACCATCATCTTTACCGAGCATAGAAGAGCTACACCAAAACCCTGGGTCATCAGCCAGCAC
ATACACTTATCCAGTGATAAATACACATCATCGGGTGCCTACATACATACCTGAATATAAAA
AAAATACTTTTGCTGAGATGAAACAGGCGTGATTTATTTCAAATAGGTACGGATAAGTAGAT
ATTGAAGTAAGGATTCAGTCTTATATTATATTACATAACATTAATCTATTCCTGCACTGAAA
CTGTTGCTTTATAGGATTTTTCACTACACTAATGAGAACTTAAGAGATAATGGCCTAAAACC
ACAGAGAGTATATTCAAAGATAAGTATAGCACTTCTTATTTGGAAACCAATGCTTACTAAAT
GAGACTAAGACGTGTCCCATCAAAAATCCTGGACCTATGCCTAAAACACATTTCACAATCCC
TGAACTTTTCAAAAATTGGTACATGCTTTAACTTTAAACTACAGGCCTCACTGGAGCTACAG
ACAAGAAGGTGAAAAACGGCTGACAAAAGAAGTCCTGGTATCTTCTATGGTGGGAGAAGAAA
ACTAGCTAAAGGGAAGAATAAATTAGAGAAAAATTGGAATGACTGAATCGGAACAAGGCAAA
GGCTATAAAAAAAATTAAGCAGCAGTATCCTCTTGGGGGCCCCTTCCCCACACTATCTCAAT
GCAAATATCTGTCTGAAACGGTCCCTGGCTAAACTCCACCCATGGGTTGGCCAGCCTTGCCT
TGACCAATAGCCTTGACAAGGCAAACTTGACCAATAGTCTTAGAGTATCCAGTGAGGCCAGG
GGCCGGCGGCTGGCTAGGGATGAAGAATAAAAGGAAGCACCCTTCAGCAGTTCCACACACTC
GCTTCTGGAACGTCTGAGGTTATCAATAAGCTCCTAGTCCAGACGCCATGGGTCATTTCACA
GAGGAGGACAAGGCTACTATCACAAGCCTGTGGGGCAAGGTGAATGTGGAAGATGCTGGAGG
AGAAACCCTGGGAAGGTAGGCTCTGGTGACCAGGACAAGGGAGGGAAGGAAGGACCCTGTGC
CTGGCAAAAGTCCAGGTCGCTTCTCAGGATTTGTGGCACCTTCTGACTGTCAAACTGTTCTT
GTCAATCTCACAGGCTCCTGGTTGTCTACCCATGGACCCAGAGGTTCTTTGACAGCTTTGGC
AACCTGTCCTCTGCCTCTGCCATCATGGGCAACCCCAAAGTCAAGGCACATGGCAAGAAGGT
GCTGACTTCCTTGGGAGATGCCATAAAGCACCTGGATGATCTCAAGGGCACCTTTGCCCAGC
TGAGTGAACTGCACTGTGACAAGCTGCATGTGGATCCTGAGAACTTCAAGGTGAGTCCAGGA
GATGTTTCAGCACTGTTGCCTTTAGTCTCGAGGCAACTTAGACAACTGAGTATTGATCTGAG
CACAGCAGGGTGTGAGCTGTTTGAAGATACTGGGGTTGGGAGTGAAGAAACTGCAGAGGACT
AACTGGGCTGAGACCCAGTGGCAATGTTTTAGGGCCTAAGGAGTGCCTCTGAAAATCTAGAT
GGACAACTTTGACTTTGAGAAAAGAGAGGTGGAAATGAGGAAAATGACTTTTCTTTATTAGA
TTTCGGTAGAAAGAACTTTCACCTTTCCCCTATTTTTGTTATTCGTTTTAAAACATCTATCT
GGAGGCAGGACAAGTATGGTCATTAAAAAGATGCAGGCAGAAGGCATATATTGGCTCAGTCA
AAGTGGGGAACTTTGGTGGCCAAACATACATTGCTAAGGCTATTCCTATATCAGCTGGACAC
ATATAAAATGCTGCTAATGCTTCATTACAAACTTATATCCTTTAATTCCAGATGGGGGCAAA
GTATGTCCAGGGGTGAGGAACAATTGAAACATTTGGGCTGGAGTAGATTTTGAAAGTCAGCT
CTGTGTGTGTGTGTGTGTGTGTGCGCGCGTGTGTTTGTGTGTGTGTGAGAGCGTGTGTTTCT
TTTAACGTTTTCAGCCTACAGCATACAGGGTTCATGGTGGCAAGAAGATAACAAGATTTAAA
TTATGGCCAGTGACTAGTGCTGCAAGAAGAACAACTACCTGCATTTAATGGGAAAGCAAAAT
CTCAGGCTTTGAGGGAAGTTAACATAGGCTTGATTCTGGGTGGAAGCTTGGTGTGTAGTTAT
CTGGAGGCCAGGCTGGAGCTCTCAGCTCACTATGGGTTCATCTTTATTGTCTCCTTTCATCT
CAACAGCTCCTGGGAAATGTGCTGGTGACCGTTTTGGCAATCCATTTCGGCAAAGAATTCAC
CCCTGAGGTGCAGGCTTCCTGGCAGAAGATGGTGACTGGAGTGGCCAGTGCCCTGTCCTCCA
GATACCACTGAGCTCACTGCCCATGATGCAGAGCTTTCAAGGATAGGCTTTATTCTGCAAGC
AATCAAATAATAAATCTATTCTGCTAAGAGATCACACATGGTTGTCTTCAGTTCTTTTTTTA
TGTCTTTTTAAATATATGAGCCACAAAGGGTTTTATGTTGAGGGATGTGTTTATGTGTATTT
ATACATGGCTATGTGTGTTTGTGTCATGTGCACACTCCACACTTTTTTGTTTACGTTAGATG
TGGGTTTTGATGAGCAAATAAAAGAACTAGGCAATAAAGAAACTTGTACATGGGAGTTCTGC
AAGTGGGAGTAAAAGGTGCAGGAGAAATCTGGTTGGAAGAAAGACCTCTATAGGACAGGACT
CCTCAGAAACAGATGTTTTGGAAGAGATGGGGAAAGGTTCAGTGAAGGGGGCTGAACCCCCT
TCCCTGGATTGCAGCACAGCAGCGAGGAAGGGGCTCAACGAAGAAAAAGTGTTCCAAGCTTT
AGGAAGTCAAGGTTTAGGCAGGGATAGCCATTCTATTTTATTAGGGGCAATACTATTTCCAA
CGGCATCTGGCTTTTCTCAGCCCTTGTGAGGCTCTACAGGGAGGTTGAGGTGTTAGAGATCA
GAGCAGGAAACAGGTTTTTCTTTCCACGGTAACTACAATGAAGTGATCCTTACTTTACTAAG
GAACTTTTCATTTTAAGTGTTGACGCATGCCTAAAGAGGTGAAATTAATCCCATACCCTTAA
GTCTACAGACTGGTCACAGCATTTCAAGGAGGAGACCTCATTGTAAGCTTCTAGGGAGGTGG
GGACTTAGGTGAAGGAAATGAGCCAGCAGAAGCTCACAAGTCAGCATCAGCGTGTCATGTCT
CAGCAGCAGAACAGCACGGTCAGATGAAAATATAGTGTGAAGAATTTGTATAACATTAATTG
AGAAGGCAGATTCACTGGAGTTCTTATATAATTGAAAGTTAATGCACGTTAATAAGCAAGAG
TTTAGTTTAATGTGATGGTGTTATGAACTTAACGCTTGTGTCTCCAGAAAATTCACATGCTG
AATCCCCAACTCCCAATTGGCTCCATTTGTGGGGGAGGCTTTGGAAAAGTAATCAGGTTTAG
AGGAGCTCATGAGAGCAGATCCCCATCATAGAATTATTTTCCTCATCAGAAGCAGAGAGATT
AGCCATTTCTCTTCCTTCTGGTGAGGACACAGTGGGAAGTCAGCCACCTGCAACCCAGGAAG
AGAGCCCTGACCAGGAACCAGCAGAAAAGTGAGAAAAAATCCTGTTGTTGAAGTCACCCAGT
CTATGCTATTTTGTTATAGCACCTTGCACTAAGTAAGGCAGATGAAGAAAGAGAAAAAAATA
AGCTTCGGTGTTCAGTGGATTAGAAACCATGTTTATCTCAGGTTTACAAATCTCCACTTGTC
CTCTGTGTTTCAGAATAAAATACCAACTCTACTACTCTCATCTGTAAGATGCAAATAGTAAG
CCTGAGCCCTTCTGTCTAACTTTGAATTCTATTTTTTCTTCAACGTACTTTAGGCTTGTAAT
GTGTTTATATACAGTGAAATGTCAAGTTCTTTCTTTATATTTCTTTCTTTCTTTTTTTTCCT
CAGCCTCAGAGTTTTCCACATGCCCTTCCTACTTTCAGGAACTTCTTTCTCCAAACGTCTTC
TGCCTGGCTCCATCAAATCATAAAGGACCCACTTCAAATGCCATCACTCACTACCATTTCAC
AATTCGCACTTTCTTTCTTTGTCCTTTTTTTTTTTAGTAAAACAAGTTTATAAAAAATTGAA
GGAATAAATGAATGGCTACTTCATAGGCAGAGTAGACGCAAGGGCTACTGGTTGCCGATTTT
TATTGTTATTTTTCAATAGTATGCTAAACAAGGGGTAGATTATTTATGCTGCCCATTTTTAG
ACCATAAAAGATAACTTCCTGATGTTGCCATGGCATTTTTTTCCTTTTAATTTTATTTCATT
TCATTTTAATTTCGAAGGTACATGTGCAGGATGTGCAGGCTTGTTACATGGGTAAATGTGTG
TCTTTCTGGCCTTTTAGCCATCTGTATCAATGAGCAGATATAAGCTTTACACAGGATCATGA
AGGATGAAAGAATTTCACCAATATTATAATAATTTCAATCAACCTGATAGCTTAGGGGATAA
ACTAATTTGAAGATACAGCTTGCCTCCGATAAGCCAGAATTCCAGAGCTTCTGGCATTATAA
TCTAGCAAGGTTAGAGATCATGGATCACTTTCAGAGAAAAACAAAAACAAACTAACCAAAAG
CAAAACAGAACCAAAAAACCACCATAAATACTTCCTACCCTGTTAATGGTCCAATATGTCAG
AAACAGCACTGTGTTAGAAATAAAGCTGTCTAAAGTACACTAATATTCGAGTTATAATAGTG
TGTGGACTATTAGTCAATAAAAACAACCCTTGCCTCTTTAGAGTTGTTTTCCATGTACACGC
ACATCTTATGTCTTAGAGTAAGATTCCCTGAGAAGTGAACCTAGCATTTATACAAGATAATT
AATTCTAATCCACAGTACCTGCCAAAGAACATTCTACCATCATCTTTACTGAGCATAGAAGA
GCTACGCCAAAACCCTGGGTCATCAGCCAGCACACACACTTATCCAGTGGTAAATACACATC
ATCTGGTGTATACATACATACCTGAATATGGAATCAAATATTTTTCTAAGATGAAACAGTCA
TGATTTATTTCAAATAGGTACGGATAAGTAGATATTGAGGTAAGCATTAGGTCTTATATTAT
GTAACACTAATCTATTACTGCGCTGAAACTGTGGCTTTATAGAAATTGTTTTCACTGCACTA
TTGAGAAATTAAGAGATAATGGCAAAAGTCACAAAGAGTATATTCAAAAAGAAGTATAGCAC
TTTTTCCTTAGAAACCACTGCTAACTGAAAGAGACTAAGATTTGTCCCGTCAAAAATCCTGG
ACCTATGCCTAAAACACATTTCACAATCCCTGAACTTTTCAAAAATTGGTACATGCTTTAGC
TTTAAACTACAGGCCTCACTGGAGCTAGAGACAAGAAGGTAAAAAACGGCTGACAAAAGAAG
TCCTGGTATCCTCTATGATGGGAGAAGGAAACTAGCTAAAGGGAAGAATAAATTAGAGAAAA
ACTGGAATGACTGAATCGGAACAAGGCAAAGGCTATAAAAAAAATTAGCAGTATCCTCTTGG
GGGCCCCTTCCCCACACTATCTCAATGCAAATATCTGTCTGAAACGGTCCCTGGCTAAACTC
CACCCATGGGTTGGCCAGCCTTGCCTTGACCAATAGCCTTGACAAGGCAAACTTGACCAATA
GTCTTAGAGTATCCAGTGAGGCCAGGGGCCGGCGGCTGGCTAGGGATGAAGAATAAAAGGAA
GCACCCTTCAGCAGTTCCACACACTCGCTTCTGGAACGTCTGAGGTTATCAATAAGCTCCTA
GTCCAGACGCCATGGGTCATTTCACAGAGGAGGACAAGGCTACTATCACAAGCCTGTGGGGC
AAGGTGAATGTGGAAGATGCTGGAGGAGAAACCCTGGGAAGGTAGGCTCTGGTGACCAGGAC
AAGGGAGGGAAGGAAGGACCCTGTGCCTGGCAAAAGTCCAGGTCGCTTCTCAGGATTTGTGG
CACCTTCTGACTGTCAAACTGTTCTTGTCAATCTCACAGGCTCCTGGTTGTCTACCCATGGA
CCCAGAGGTTCTTTGACAGCTTTGGCAACCTGTCCTCTGCCTCTGCCATCATGGGCAACCCC
AAAGTCAAGGCACATGGCAAGAAGGTGCTGACTTCCTTGGGAGATGCCACAAAGCACCTGGA
TGATCTCAAGGGCACCTTTGCCCAGCTGAGTGAACTGCACTGTGACAAGCTGCATGTGGATC
CTGAGAACTTCAAGGTGAGTCCAGGAGATGTTTCAGCCCTGTTGCCTTTAGTCTCGAGGCAA
CTTAGACAACGGAGTATTGATCTGAGCACAGCAGGGTGTGAGCTGTTTGAAGATACTGGGGT
TGGGGGTGAAGAAACTGCAGAGGACTAACTGGGCTGAGACCCAGTGGTAATGTTTTAGGGCC
TAAGGAGTGCCTCTAAAAATCTAGATGGACAATTTTGACTTTGAGAAAAGAGAGGTGGAAAT
GAGGAAAATGACTTTTCTTTATTAGATTCCAGTAGAAAGAACTTTCATCTTTCCCTCATTTT
TGTTGTTTTAAAACATCTATCTGGAGGCAGGACAAGTATGGTCGTTAAAAAGATGCAGGCAG
AAGGCATATATTGGCTCAGTCAAAGTGGGGAACTTTGGTGGCCAAACATACATTGCTAAGGC
TATTCCTATATCAGCTGGACACATATAAAATGCTGCTAATGCTTCATTACAAACTTATATCC
TTTAATTCCAGATGGGGGCAAAGTATGTCCAGGGGTGAGGAACAATTGAAACATTTGGGCTG
GAGTAGATTTTGAAAGTCAGCTCTGTGTGTGTGTGTGTGTGTGCGCGCGCGCGTGTGTGTGT
GTGTGTCAGCGTGTGTTTCTTTTAACGTCTTCAGCCTACAACATACAGGGTTCATGGTGGCA
AGAAGATAGCAAGATTTAAATTATGGCCAGTGACTAGTGCTTGAAGGGGAACAACTACCTGC
ATTTAATGGGAAGGCAAAATCTCAGGCTTTGAGGGAAGTTAACATAGGCTTGATTCTGGGTG
GAAGCTTGGTGTGTAGTTATCTGGAGGCCAGGCTGGAGCTCTCAGCTCACTATGGGTTCATC
TTTATTGTCTCCTTTCATCTCAACAGCTCCTGGGAAATGTGCTGGTGACCGTTTTGGCAATC
CATTTCGGCAAAGAATTCACCCCTGAGGTGCAGGCTTCCTGGCAGAAGATGGTGACTGCAGT
GGCCAGTGCCCTGTCCTCCAGATACCACTGAGCTCACTGCCCATGATTCAGAGCTTTCAAGG
ATAGGCTTTATTCTGCAAGCAATACAAATAATAAATCTATTCTGCTGAGAGATCACACATGA
TTTTCTTCAGCTCTTTTTTTTACATCTTTTTAAATATATGAGCCACAAAGGGTTTATATTGA
GGGAAGTGTGTATGTGTATTTCTGCATGCCTGTTTGTGTTTGTGGTGTGTGCATGCTCCTCA
TTTATTTTTATATGAGATGTGCATTTTGATGAGCAAATAAAAGCAGTAAAGACACTTGTACA
CGGGAGTTCTGCAAGTGGGAGTAAATGGTGTAGGAG.

By “hemoglobin, gamma G (HBG2) polypeptide” is meant a polypeptide having at least about 85% amino acid sequence identity to Genbank Accession No. CAA23773.1, provided below, or a fragment thereof capable of forming a protein complex with alpha hemoglobin subunits.

(SEQ ID NO: 431)
MGHFTEEDKATITSLWGKVNVEDAGGETLGRLLVVYPWTQRFFDSFGNL
SSASAIMGNPKVKAHGKKVLTSLGDAIKHLDDLKGTFAQLSELHCDKLH
VDPENFKLLGNVLVTVLAIHFGKEFTPEVQASWQKMVTGVASALSSRYH.

By HBG2 polynucleotide” is meant a nucleic acid molecule that encodes an HBG2 polypeptide as well as the introns, exons, 3′ untranslated regions, 5′ untranslated regions, and regulatory sequences associated with its expression, or fragments thereof. In embodiments, a HBG2 polynucleotide is the genomic sequence, cDNA, mRNA, or gene associated with and/or required for HBG2 expression. An exemplary HBB polynucleotide sequence from Homo sapiens is provided on ENSEMBL at accession no. GRCh38:11:5248044:5259425:1, the reverse-complement of which is provided above (SEQ ID NO: 430). In the above sequence (SEQ ID NO: 430), exons encoding HBG2 are shown in bold-underlined text and an exemplary HBG2 promoter region corresponds to a region 5′ of the first exon encoding HBG2 (e.g., the first 100, 200, 300, or 400 nucleotides 5′ of the first exon), or portions thereof.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

By “chimeric antigen receptor T cell” or “CAR-T cell” is meant a T cell expressing a CAR that has antigen specificity determined by the antibody-derived targeting domain of the CAR. As used herein, “CAR-T cells” include regulatory T (TREG) cells. As used herein, “CAR-T cells” include cells engineered to express a CAR or a T cell receptor (TCR). Methods of making CARs are publicly available (see, e.g., Park et al., Trends Biotechnol., 29:550-557, 2011; Grupp et al., N Engl J Med., 368:1509-1518, 2013; Han et al., J. Hematol Oncol. 6:47, 2013; Haso et al., (2013) Blood, 121, 1165-1174; Mohseni, et al., (2020) Front. Immunol., 11, art. 1608, doi: 10.3389/fimmu.2020.01608; Eggenhuizen, et al. Int. J. Mol. Sci. (2020), 21:7015, doi: 10.3390/ijms21197015; PCT Pubs. WO2012/079000, WO2013/059593; and U.S. Pub. 2012/0213783, the disclosure of each of which is incorporated herein by reference herein in its entirety for all purposes).

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

As used herein, the terms “condition” and “conditioning” refer to processes by which a patient is prepared for receipt of a transplant containing hematopoietic stem cells. Such procedures promote the engraftment of a hematopoietic stem cell transplant (for instance, as inferred from a sustained increase in the quantity of viable hematopoietic stem cells within a blood sample isolated from a patient following a conditioning procedure and subsequent hematopoietic stem cell transplantation). According to the methods described herein, a patient may be conditioned for hematopoietic stem cell transplant therapy by administration to the patient of an antibody or antigen-binding fragment thereof capable of binding an antigen expressed by hematopoietic stem cells, such as CD117, CXCR4, CD135, CD90, CD45, and/or CD34. Such antibodies are expected to act via complement-mediated cytotoxicity and antibody-dependent cell-mediated cytotoxicity. As described herein, the transplanted cells have been edited so that the antibody no longer binds the antigen (e.g., CD117, CXCR4, CD135, CD90, CD45, and/or CD34). Administration of an antibody, antigen-binding fragment thereof, drug-antibody conjugate, or chimeric antigen receptor expressing T-cell (CAR-T) capable of binding one or more antigens (e.g., CD117, CXCR4, CD135, CD90, CD45, CD34) to a patient in need of hematopoietic stem cell transplant therapy can promote the engraftment of a hematopoietic stem cell graft, for example, by selectively depleting endogenous hematopoietic stem cells, thereby creating a vacancy filled by an exogenous hematopoietic stem cell transplant.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

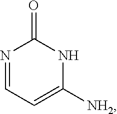

and corresponding to CAS No. 71-30-7.

By “cytidine” is meant a cytosine molecule attached to a ribose sugar via a glycosidic bond, having the structure

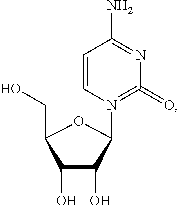

and corresponding to CAS No. 65-46-3. Its molecular formula is C₉H₁₃N₃O₅.

The term “deaminase” or “deaminase domain,” as used herein, refers to a protein or fragment thereof that catalyzes a deamination reaction.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. In an embodiment, the disease is sickle cell disease or ß-thalassemia.

By “effective amount” is meant the amount of an agent (e.g., a base editor, cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

As used herein, the term “hematopoietic stem cells” (“HSCs”) refers to immature blood cells having the capacity to self-renew and to differentiate into mature blood cells containing diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Such cells may include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSCs (LT-HSC) and short term repopulating HSCs (ST-HSC). LT-HSCs and ST-HSCs are differentiated, based on functional potential and on cell surface marker expression. For example, human HSCs are CD34+, CD38−, CD45RA−, CD90+, CD49F+, and lin-(negative for mature lineage markers including CD2, CD3, CD4, CD7, CD8, CD10, CD11B, CD19, CD20, CD56, CD235A). In mice, bone marrow LT-HSCs are CD34−, SCA-1+, C-kit+, CD135−, Slamfl/CD150+, CD48−, and lin-(negative for mature lineage markers including Ter119, CD11b, Gr1, CD3, CD4, CD8, B220, IL7ra), whereas ST-HSCs are CD34+, SCA-1+, C-kit+, CD135−, Slamfl/CD150+, and lin-(negative for mature lineage markers including Ter1 19, CD11b, Gr1, CD3, CD4, CD8, B220, IL7ra). In addition, ST-HSCs are less quiescent and more proliferative than LT-HSCs under homeostatic conditions. However, LT-HSC have greater self-renewal potential (i.e., they survive throughout adulthood, and can be serially transplanted through successive recipients), whereas ST-HSCs have limited self-renewal (i.e., they survive for only a limited period of time, and do not possess serial transplantation potential). Any of these HSCs can be used in the methods described herein. ST-HSCs are particularly useful because they are highly proliferative and thus, can more quickly give rise to differentiated progeny.

As used herein, the term “hematopoietic stem cell functional potential” refers to the functional properties of hematopoietic stem cells which include 1) multi-potency (which refers to the ability to differentiate into multiple different blood lineages including, but not limited to, granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells), 2) self-renewal (which refers to the ability of hematopoietic stem cells to give rise to daughter cells that have equivalent potential as the mother cell, and further that this ability can repeatedly occur throughout the lifetime of an individual without exhaustion), and 3) the ability of hematopoietic stem cells or progeny thereof to be reintroduced into a transplant recipient whereupon they home to the hematopoietic stem cell niche and re-establish productive and sustained hematopoiesis.”

By “heterologous,” or “exogenous” is meant a polynucleotide or polypeptide that 1) has been experimentally incorporated to a polynucleotide or polypeptide sequence to which the polynucleotide or polypeptide is not normally found in nature; or 2) has been experimentally placed into a cell that does not normally comprise the polynucleotide or polypeptide. In some embodiments, “heterologous” means that a polynucleotide or polypeptide has been experimentally placed into a non-native context. In some embodiments, a heterologous polynucleotide or polypeptide is derived from a first species or host organism and is incorporated into a polynucleotide or polypeptide derived from a second species or host organism. In some embodiments, the first species or host organism is different from the second species or host organism. In some embodiments the heterologous polynucleotide is DNA. In some embodiments the heterologous polynucleotide is RNA.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

“Makassar” or “Hb G-Makassar” refers to a human β-hemoglobin variant, the human Hemoglobin (Hb) of G-Makassar variant or mutation (HB Makassar variant), which is an asymptomatic, naturally-occurring variant (E6A) hemoglobin. Hb G-Makassar was first identified in Indonesia. (Mohamad, A. S. et al., 2018, Hematol. Rep., 10(3):7210 (doi:10.4081/hr.2018.7210). The Hb G-Makassar mobility is slower when subjected to electrophoresis. The Makassar β-hemoglobin variant has its anatomical abnormality at the 0-6 or A3 location where the glutamyl residue typically is replaced by an alanyl residue. The substitution of single amino acid in the gene encoding the β-globin subunit 0-6 glutamyl to valine will result as sickle cell disease. Routine procedures, such as isoelectric focusing, hemoglobin electrophoresis separation by cation-exchange High Performance Liquid Chromatography (HPLC) and cellulose acetate electrophoresis, have been unable to separate the Hb G-Makassar and HbS globin forms, as they were found to have identical properties when analyzed by these methods. Consequently, Hb G-Makassar and HbS have been incorrectly identified and mistaken for each other by those skilled in the art, thus leading to misdiagnosis of Sickle Cell Disease (SCD). In one embodiment, the valine at amino acid position 6, which causes sickle cell disease, is replaced with an alanine, to thereby generate an Hb variant (Hb Makassar) that does not generate a sickle cell phenotype. In some embodiments, a Val→Ala (GTG→GCG) replacement (i.e., the Hb Makassar variant) can be generated using an A•T to G•C base editor (ABE).

By “marker” is meant any protein or polynucleotide having an alteration in expression, level, structure, or activity that is associated with a disease or disorder. In some embodiments, a marker is single-nucleotide polymorphism associated with a disease or disorder.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt.4172. In some embodiments, an NLS comprises the amino acid sequence KRTADGSEFESPKKKRKV (SEQ ID NO: 190), KRPAATKKAGQAKKKK (SEQ ID NO: 191), KKTELQTTNAENKTKKL (SEQ ID NO: 192), KRGINDRNFWRGENGRKTR (SEQ ID NO: 193), RKSGKIAAIVVKRPRK (SEQ ID NO: 194), PKKKRKV (SEQ ID NO: 195), MDSLLMNRRKFLYQFKNVRWAKGRRETYLC (SEQ ID NO: 196), PKKKRKVEGADKRTADGSE FESPKKKRKV (SEQ ID NO: 328), or RKSGKIAAIVVKRPRKPKKKRKV (SEQ ID NO: 329).

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-245, 254-260, and 378.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase).

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the reference is a wild-type or healthy cell. In other embodiments and without limitation, a reference is an untreated cell that is not subjected to a test condition, or is subjected to placebo or normal saline, medium, buffer, and/or a control vector that does not harbor a polynucleotide of interest. In some cases, a “reference” is an untreated subject, such as a subject not administered a hematopoietic stem cell edited according to the methods of the present disclosure. In some cases the subject is a healthy subject (e.g., a subject not having sickle cell disease). In some embodiments, the reference is an unedited or wild type cell, polypeptide, or polynucleotide.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csnl) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9).

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “single nucleotide polymorphism (SNP)” is a variation in a single nucleotide that occurs at a specific position in the genome, where each variation is present to some appreciable degree within a population (e.g., >1%). For example, at a specific base position in the human genome, the C nucleotide can appear in most individuals, but in a minority of individuals, the position is occupied by an A. This means that there is a SNP at this specific position, and the two possible nucleotide variations, C or A, are said to be alleles for this position. SNPs underlie differences in susceptibility to disease. The severity of illness and the way our body responds to treatments are also manifestations of genetic variations. SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “selectively binds” is meant specifically binds a wild type version of the cell surface protein but exhibits reduced binding or fails to detectably bind to the cell surface protein comprising a mutation. In embodiments, an antibody of the present disclosure selectively binds to a wild type CD117 polypeptide but exhibits reduced binding to a CD117 polypeptide comprising one or more amino acid alterations, such as those provided herein, relative to the wild type CD117 polypeptide. In embodiments, an antibody of the present disclosure binds a wild type CD117 polypeptide 1.1-fold, 1.2-fold, 1.3-fold, 1.4-fold, 1-fold, 5-fold, 1.75-fold, 2-fold, 3-fold, 4-fold, 5-fold, 6-fold, 7-fold, 8-fold, 9-fold, 10-fold, 100-fold, 1000-fold, 10000-fold, 100000-fold, or 1000000-fold more strongly (e.g., as quantified using KD(M), where a lower KD(M) indicates stronger binding) than to an altered CD117 polypeptide of the present disclosure.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

The term “target site” refers to a nucleotide sequence or nucleobase of interest within a nucleic acid molecule that is modified. In embodiments, the modification is deamination of a base. The deaminase can be an adenine deaminase. The fusion protein or base editing complex comprising a deaminase may comprise a dCas9-adenosine deaminase fusion protein, a Cas12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein. In some embodiments, the disease or disorder is sickle cell disease (SCD) or ß-thalassemia.

As used herein, the term “vector” refers to a means of introducing a nucleic acid molecule into a cell, resulting in a transformed cell. Vectors include plasmids, transposons, phages, viruses, liposomes, lipid nanoparticles, and episomes. “Expression vectors” are nucleic acid sequences comprising the nucleotide sequence to be expressed in the recipient cell. Expression vectors contain a polynucleotide sequence as well as additional nucleic acid sequences to promote and/or facilitate the expression of the introduced sequence, such as start, stop, enhancer, promoter, and secretion sequences, into the genome of a mammalian cell. Examples of vectors include nucleic acid vectors, e.g., DNA vectors, such as plasmids, RNA vectors, viruses or other suitable replicons (e.g., viral vectors). A variety of vectors have been developed for the delivery of polynucleotides encoding exogenous proteins into a prokaryotic or eukaryotic cell. Examples of such expression vectors are disclosed in, e.g., WO 1994/11026; incorporated herein by reference. Certain vectors that can be used for the expression of antibodies and antibody fragments of some aspects and embodiments herein include plasmids that contain regulatory sequences, such as promoter and enhancer regions, which direct gene transcription. Other useful vectors for expression of antibodies and antibody fragments contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector. The expression vectors of some aspects and embodiments herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIGS. 1A-1C depict plasmids. FIG. 1A is a map of an expression vector encoding a TadA7.10-dCas9 base editor. FIG. 1B is a map of a plasmid comprising nucleic acid molecules encoding proteins that confer chloramphenicol resistance (CamR) and spectinomycin resistance (SpectR). The plasmid also comprises a kanamycin resistance gene disabled by two point mutations. FIG. 1C is a map of a plasmid comprising nucleic acid molecules encoding proteins that confer chloramphenicol resistance (CamR) and spectinomycin resistance (SpectR). The plasmid also comprises a kanamycin resistance gene disabled by three point mutations.

FIG. 2 presents images of bacterial colonies transduced with the expression vectors depicted in FIG. 1, which included a defective kanamycin resistance gene. The vectors contained ABE7.10 variants that were generated using error prone PCR. Bacterial cells expressing these “evolved” ABE7.10 variants were selected for kanamycin resistance using increasing concentrations of kanamycin. Bacteria expressing ABE7.10 variants having adenosine deaminase activity were capable of correcting the mutations introduced into the kanamycin resistance gene, thereby restoring kanamycin resistance. The kanamycin resistant cells were selected for further analysis.

FIGS. 3A and 3B illustrate editing of a regulatory region of the hemoglobin subunit gamma (HBG1) locus, which is a therapeutically relevant site for upregulation of fetal hemoglobin. FIG. 3B is a schematic of a portion of the regulatory region for the HBG1 gene. FIG. 3A discloses SEQ ID NO: 432. FIG. 3A provides a bar graph that quantifies the efficiency and specificity of adenosine deaminase variants listed in Table 15. Editing was assayed at the hemoglobin subunit gamma 1 (HBG1) locus in HEK293T cells, which was therapeutically relevant site for upregulation of fetal hemoglobin. The top panel depicts nucleotide residues in the target region of the regulatory sequence of the HBG1 gene. A5, A8, A9, and A11 denote the edited adenosine residues in HBG1, as indicated above the target sequence shown in FIG. 3A. FIG. 3B discloses SEQ ID NOs: 433 and 434, where SEQ ID NO: 433 and 434 are reverse complements of one another.

FIG. 4 illustrates the relative effectiveness of adenosine base editors comprising a dCas9 that recognizes a noncanonical PAM sequence. The top panel depicts the coding sequence of the hemoglobin subunit. The bottom panel is a graph demonstrating the efficiency of adenosine deaminase variant base editors with guide RNAs of varying lengths. FIG. 4 discloses SEQ ID NOs: 435 and 437 respectively, in order of appearance.

FIG. 5 is a bar graph illustrating the efficiency and specificity of ABE8s. The percent editing at intended target nucleotides and unintended target nucleotides (bystanders) is quantified. FIG. 5 discloses SEQ ID NOs: 438 and 435, respectively, in order of appearance.

FIG. 6 is a bar graph illustrating the efficiency and specificity of ABE8s. The percent editing at intended target nucleotides and unintended target nucleotides (bystanders) is quantified.

FIG. 7 provides a stacked bar graph showing percent target, bystander, and non-synonymous bystander A>G edits corresponding to the indicated base editors. In FIG. 7 each bar indicates from top-to-bottom “other non-synonymous bystanders”, “1G bystander”, and “favorable” edits. In FIG. 7 “XVIVO” refers to the serum free stem cell medium in which cells were grown, “IVD” refers to “in vitro differentiated erythroid cultures (IVD)”, and “d5” and “d7” refer to five days and seven days, respectively.

FIG. 8 provides a stacked bar graph showing editing efficiencies (A to G %) for the base editors ABE8.20-NRCH (1570), ABE9v1-NRCH (2517), and ABE9v2 (2518) used in combination with the guide gRNA931. In each stacked bar of FIG. 8, the following edits are shown in order from top-to-bottom: “3G_4G_6G,” “4G_6G,” “3G_6G,” and “6G.”

FIG. 9 provides histograms showing that cells expressing CD117 polypeptides altered using ABE8.20-NRCH (1570), ABE9v1-NRCH (2517), or ABE9v2 (2518) in combination with the guide gRNA931 showed reduced binding to the antibody ABTx052. Cells were evaluated 2 days post-electroporation (EP). In FIG. 9, “1” indicates cells edited using ABE8.20-NRCH, “2” indicates cells edited using ABE9v1-NRCH, and “3” indicates cells edited using ABE9v2.

FIG. 10 provides a bar graph showing engraftment of base-edited hCD34+ in NBSGW mice at 16-weeks post-transplantation. The bar graph of FIG. 10 shows the percent of each indicated hCD34+ cell type that contained base edited beta globin polynucleotides containing one of the following base modifications (i.e., “Makassar Conversion (%)”): 9G, 9G+11G, 9G+11G+14G, 5T+9G+11G, where the numbers indicate the nucleotide location of each alteration, and where each alteration is indicated relative to the following sequence, where subscripts indicate the nucleotide locations: ACTTC₅TCCA₉CA₁₁GGA₁₄GTCAGATGC (SEQ ID NO: 439). In FIG. 10, the leftmost five bars correspond to mice transplanted with unedited CD34+ cells and the rightmost five bars correspond to mice transplanted with base edited CD34+ cells.

FIGS. 11A-11G provide bar graphs showing engraftment and multilineage reconstitution at 16 weeks post-hCD34+ cell transplant in immunocompromised NBSGW (NOD.Cg-Kit^{W-41J} Tyr⁺ Prkdc^{scid} Il2rg^{tm1Wjl}/ThomJ) mice (6-8 weeks, female). The mice were transplanted with 1e6 base edited or unedited hCD34+ cells. At 16 weeks post transplantation, mice were euthanized, and bone marrow was harvested from the pelvic bones, tibias and femurs. Frequency of cell populations were determined using flow cytometry. Bulk bone marrow (BM) cells were stained with reagents against hCD45, mCD45, hCD34, hCD33, hCD15, hCD19, GlyA, hCD16, hCD56, and LIVE/DEAD Fixable Aqua Dead Cell Stain Kit. Base editing did not alter engraftment of the hCD34+ cells or multilineage reconstitution potential.

### DETAILED DESCRIPTION

The disclosure features adenosine deaminase base editors, compositions comprising the same, and methods for use thereof for altering a target nucleobase in a polynucleotide sequence. In some embodiments, the base editors of the disclosure may be used to treat a disease or disorder, such as a hemoglobinopathy (e.g., sickle cell disease (SCD)).

The aspects of the disclosure and embodiments thereof are based, at least in part, on the discovery, as described in the Examples provided herein, of new adenosine deaminase variants useful in altering a nucleobase of a polynucleotide sequence. Accordingly, the disclosure provides new adenosine deaminase variants useful in the methods provided herein for altering polynucleotide sequences.

### Cluster of Differentiation 117 (CD117; C-Kit)

CD117 is expressed in hematopoietic stem cells (HSCs) and is critical for their self-renewal, survival & differentiation. Upon differentiation, CD117 expression is lost. Mature mast cells retain CD117 expression. High level of expression the long term and short-term HSCs make CD117 an attractive target for immunologic conditioning.

Therefore, one approach to eliminate hematopoietic stem cells from a niche is to contact the cells with an anti-CD117 antibody that interferes with proper functioning of the CD117 polypeptide (e.g., blocks binding to SCF). Accordingly, CD117/c-KIT is a target for hematopoietic stem cell transplantation (HSCT) antibody-based conditioning. Non-limiting examples of antibodies suitable for use in the methods of the disclosure include ABTx052. In embodiments, a CD117 variant prepared according to the methods of the disclosure has reduced binding to ABTx052 relative to a wild type CD117 polypeptide.

Critical events in the DCD117 life cycle include SCF binding, CD117 homo-dimerization, trans-phosphorylation of tyrosine residues, ubiquitinization, internalization, and proteolytic degradation. Trans-phosphorylation of the tyrosine residues is associated with cell activation and downstream phosphorylation, calcium mobilization, and cell migration.

### HBB Gene Editing

As described herein, the compositions and methods of the invention are useful and advantageous for the treatment of sickle cell disease (SCD), which is caused by a Glu→Val mutation at the sixth amino acid of the β-globin protein encoded by the HBB gene. Despite many developments to date in the field of gene editing, precise correction of the diseased HBB gene to revert Val→Glu is presently not achievable using either CRISPR/Cas nuclease or CRISPR/Cas base editing approaches.

Genome editing of the HBB gene to replace the affected nucleotide using a CRISPR/Cas nuclease approach requires cleavage of genomic DNA. However, cleavage of genomic DNA carries an increased risk of generating base insertions/deletions (indels), which have the potential to cause unintended and undesirable consequences, including generating premature stop codons, altering the codon reading frame, etc. Furthermore, generating double-stranded breaks at the β-globin locus has the potential to radically alter the locus through recombination events. The β-globin locus contains a cluster of globin genes having sequence identity to one another—5′-ε-; Gγ-; Aγ-; δ-; and β-globin-3′. Because of the structure of the β-globin locus, recombination repair of a double-stranded break within the locus has the potential to result in gene loss of intervening sequences between globin genes, for example between δ- and β-globin genes.

Unintended alterations to the locus also carry a risk of causing thalassemia. CRISPR/Cas base editing approaches hold promise in that they have the ability to generate precise alterations at the nucleobase level. However, precise correction of Val→Glu (GTG→→GAG) requires a T•A to A•T transversion editor, which is not presently known to exist. Additionally, the specificity of CRISPR/Cas base editing is due in part to a limited window of editable nucleotides created by R-loop formation upon CRISPR/Cas binding to DNA. Thus, CRISPR/Cas targeting must occur at or near the sickle cell site to allow base editing to be possible, and there may be additional sequence requirements for optimal editing within the window. One requirement for CRISPR/Cas targeting is the presence of a protospacer-adjacent motif (PAM) flanking the site to be targeted. For example, many base editors are based on SpCas9 which requires an NGG PAM. Even assuming hypothetically that an T•A to A•T transversion were possible, no NGG PAM exists that would place the target “A” at a desirable position for such an SpCas9 base editor. Although many new CRISPR/Cas proteins have been discovered or generated that expand the collection of available PAMs, PAM requirements remain a limiting factor in the ability to direct CRISPR/Cas base editors to specific nucleotides at any location in the genome.

The present invention is based, at least in part, on several discoveries described herein that address the foregoing challenges for providing a genome editing approach for treatment of sickle cell anemia. In one aspect, the invention is based in part on the ability to replace the valine at amino acid position 6, which causes sickle cell disease, with an alanine, to thereby generate an Hb variant (Hb Makassar) that does not generate a sickle cell phenotype. While precise correction (GTG→GAG) is not possible without a T•A to A•T transversion base editor, the studies performed herein have found that a Val→Ala (GTG→GCG) replacement (i.e., the Hb Makassar variant) can be generated using an A•T to G•C base editor (ABE). In some embodiments, the methods of the invention involve detecting an Hb Makassar variant using an antibody, such as an antibody selected from those described in U.S. Provisional Patent Application No. 63/329,109, filed Apr. 8, 2022. This was achieved in part by the development of novel base editors and novel base editing strategies, as provided herein. For example, novel ABE base editors (i.e., having an adenosine deaminase domain) that utilize flanking sequences (e.g., PAM sequences; zinc finger binding sequences) for optimal base editing at the sickle cell target site.

Thus, the present invention includes compositions and methods for base editing a thymidine (T) to a cytidine (C) in the codon of the sixth amino acid of a sickle cell disease variant of the β-globin protein (Sickle HbS; E6V), thereby substituting an alanine for a valine (V6A) at this amino acid position. Substitution of alanine for valine at position 6 of HbS generates a β-globin protein variant that does not have a sickle cell phenotype (e.g., does not have the potential to polymerize as in the case of the pathogenic variant HbS). Accordingly, the compositions and methods of the invention are useful for the treatment of sickle cell disease (SCD).

HBG1 and/or HBG2 Promoter Editing

Sickle cell disease (SCD) affects approximately 100,000 patients in the United States. Individuals carrying both the SCD mutation and mutations that cause persistence of fetal hemoglobin (HPFH) do not typically present with sickle cell pathologies due to persistent fetal hemoglobin (HbF) levels. Higher HbF levels correlate with greater benefit for individuals with blood disease, such as reduction in disease symptoms and improved overall health. A T to C mutation at the −198 position in the HBG promoter causes HPFH by interference of binding to γ-globulin repressor proteins, such as BCL11A.

Ex vivo manipulation and/or editing of HSCs prior to administration to patients as a cell therapy is a promising approach for the treatment of hematological disorders. ABEs can introduce a T to C substitution at the −198 position of the promoter region of HBG1/2 (Gaudelli, N. M. et al. Programmable base editing of A*T to G*C in genomic DNA without DNA cleavage. Nature 551, 464-471, doi:10.1038/nature24644 (2017)). This naturally occurring allele yields Hereditary Persistence of Fetal Hemoglobin (HPFH) resulting in increased levels of γ-globin into adulthood, which can mitigate the defects in β-globin seen in sickle cell disease and β-thalassemia (Wienert, B. et al. KLF1 drives the expression of fetal hemoglobin in British HPFH. Blood 130, 803-807, doi:10.1182/blood-2017-02-767400 (2017)). Base editor systems have been developed that significantly increase the level of HbF following nucleotide conversion at key regulatory motifs within the HBG1 and HBG2 (HBGT1/2) promoters.

Increasing levels of HbF expression leads to protection to the majority of SCD and ß-thalassemia patients based on clinical observations of HPFH and non-interventional therapy that links higher HbF dosage with milder disease (Ngo et al., 2011 Brit J Hem, Vol. 156(2):259-264; Musallam et al., 2012 Blood). Accordingly, in the HPFH approach described here, base editing is used to recreate single base changes in the regulatory region of both gamma globin genes (HBG1 and HBG2) that disrupt repressor binding and lead to increased expression of fetal hemoglobin (HbF). Beta-thalassemia or sickle cell disease patients naturally harboring these variants are often asymptomatic or experience a milder form of the disease.

An exemplary target sequence for introducing an alteration to an HBG1/2 promoter to increase expression of fetal hemoglobin is provided below:

### The Target Sequence, Including Edited Bases 5 and 8 (in Bold) and PAM (SEQ ID NO: 440):

| 1 | 2 | 3 | 4 | 5 | 6 | 7 | 8 | 9 | 10 | 11 | 12 | 13 | 14 | 15 | 16 | 17 | 18 | 19 | 20 | PAM |
|---|---|---|---|---|---|---|---|---|---|---|---|---|---|---|---|---|---|---|---|---|
| C | T | T | G | A | C | C | A | A | T | A | G | C | C | T | T | G | A | C | A | AGG |

### Editing of Target Genes

To produce the gene edits described above, cells (e.g., hematopoietic stem cells (HSCs)) are contacted with one or more guide RNAs and a nucleobase editor polypeptide comprising a nucleic acid programmable DNA binding protein (napDNAbp) and an adenosine deaminase. In some cases, the cells are collected from a subject prior to the contacting. In some embodiments, the gRNA comprises nucleotide analogs. These nucleotide analogs can inhibit degradation of the gRNA from cellular processes. Tables 1A, 1B, and 2 provides exemplary spacer sequences to be used for gRNAs. In some instances, a spacer sequence can be selected from those listed in Tables 1A, 1B, and 2 or from a variant thereof with a truncation and/or extension (e.g., a 1, 2, 3, 4, 5, 6, 7, 8, 9, or 10 nucleotide 3′ and/or 5′ truncation and/or extension). In some instances, the gRNA is added directly to a cell.

Base editing can be carried out in vitro or in vivo. In some embodiments, cells (e.g., a hematopoietic stem cells (HSCs)) are collected from a subject or a donor. In some embodiments, base editing is carried out to induce changes in the genome of the cell. In some embodiments, base editing is carried out to induce changes in the genome of an allogeneic cell.

In some embodiments, cells of the present disclosure, are contacted with one or more guide RNAs and a nucleobase editor polypeptide comprising a nucleic acid programmable DNA binding protein (napDNAbp) (e.g., Cas9) domain and an adenosine deaminase domain. In some embodiments, the at least one nucleic acid molecule encoding one or more guide RNAs and a nucleobase editor polypeptide is delivered to cells by one or more vectors (e.g., AAV vector or lipid nanoparticle). In some cases, a guide RNA(s) and a nucleobase editor polypeptide is delivered to cells by electroporation.

The present disclosure provides one or more guide RNAs that direct a nucleobase editor polypeptide to edit a site in the genome of the cell (e.g., hematopoietic stem cell (HSC)). In some embodiments, the present disclosure provides guide RNAs that target a CD117 polynucleotide, a beta globin (HBB) polynucleotide, and/or a promoter region of a HBG1/2 polynucleotide. Exemplary guide RNA spacer sequences are provided in the below Tables 1A, 1B, and 2.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′ “G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science.1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

In an embodiment, an end-modified guide polynucleotide contains the following nucleotide sequence:

(SEQ ID NO: 441)
mUsmUsmCsUCCACAGGAGUCAGGUGGUUUUAGAGCUAGAAAUAGCAAG
UUAAAAUAAGGCUAGUCCGUUAUCAACUUGAAAAAGUGGCACCGAGUCG
GUGCmUsmUsmUsU.

In another embodiment, a heavy mod guide polynucleotide contains the following nucleotide sequence (gRNA2861):

mUsmUsmCsUCCACAGGAGUCAGGUGGUUUUAGAmGmCmCmGmGmCmGm
GmAmAmAmCmGmCmCmGmGmCAAGUUAAAAUAAGGCUAGUCCGUUAmUm
CAAmCmUmUGGACUUCGGUCCmAmAmGUGGmCmAmCmCmGmAmGmUmCm
GmGmUmGmCmUsmUsmUsmU (SEQ ID NO: 442;
containing GOLD, LONGEST mods).

In some embodiments, a guide polynucleotide contains one of the following nucleotide sequences:

mNsmNsmNsNNNNNNNNNNNNNNNNNGUUUUAGAGCUAGAAAUAGCAAG
UUAAAAUAAGGCUAGUCCGUUAUCAACUUGAAAAAGUGGCACCGAGUCG
GUGCmUsmUsmUsU (SEQ ID NO: 443; end-modified
guide);
or
mNsmNsmNsNNNNNNNNNNNNNNNNNGUUUUAGAmGmCmCmGmGmCmGm
GmAmAmAmCmGmCmCmGmGmCAAGUUAAAAUAAGGCUAGUCCGUUAmUm
CAAmCmUmUGGACUUCGGUCCmAmAmGUGGmCmAmCmCmGmAmGmUmCm
GmGmUmGmCmUsmUsmUsmU (SEQ ID NO: 444; heavy
mod guide; containing GOLD, LONGEST mods).

Throughout the disclosure, the following notation is used to indicate chemical modifications to a guide polynucleotide: “N” represents any nucleotide, “mN” indicates a 2′-OMe modification of the nucleotide “N”, and “Ns” indicates that the nucleotide “N” is linked to the following nucleotide by a phosphorothioate (PS).

In various embodiments, a base editor system of the disclosure contains a guide polynucleotide of the disclosure (e.g., one of the above-listed guide polynucleotides) and the base editor ABE9v1 or ABE9v2.

TABLE 1A
Exemplary spacer sequences for editing HBB

| | | | | | SEQ | |
|---|---|---|---|---|---|---|
| Guide | | Description | Base | gRNA Spacer | ID | |
| Name | Gene | of Edit | Editor | Sequence | NO | PAM |
| | HBB | Makassar | ABE | UUCUCCACAGGAGUCAG | 445 | |
| | | | | GUG | | |
| sgRNA_ | HBB | Makassar | ABE | ACUUCUCCACAGGAGUC | 446 | TGC |
| 017, | | | | AGG | | |
| sgRNA_ | | | | | | |
| 027 | | | | | | |
| (TSBT | | | | | | |
| x853) | | | | | | |
| | HBB | Makassar | ABE | GACUUCUCCACAGGAGU | 447 | TGC |
| | | | | CAGG | | |
| | HBB | Makassar | ABE | CUUCUCCACAGGAGUCA | 448 | TGC |
| | | | | GG | | |
| | HBB | Makassar | ABE | CUUCUCCACAGGAGUCA | 449 | |
| | | | | GAU | | |
| | HBB | Makassar | ABE | ACUUCUCCACAGGAGUC | 450 | |
| | | | | AGAU | | |
| | HBB | Makassar | ABE | GACUUCUCCACAGGAGU | 451 | |
| | | | | CAGAU | | |
| | HBB | Makassar | ABE | UCUGACUCCUGUGGAGA | 452 | GCA |
| | | | | AGUCU | | |
| | HBB | Makassar | ABE | AGACUUCUCCACAGGAG | 453 | TBC |
| | | | | UCAGA | | |
| | HBB | Makassar | ABE | UCCACAGGAGUCAGAUG | 454 | NNNRRT |
| | | | | CAC | | (CATGGT) |

TABLE 1B
Exemplary spacer sequences for editing a promoter region of HBG1/2

| | | | | gRNA Spacer | SEQ | |
|---|---|---|---|---|---|---|
| Guide | | Description | | Sequence | ID | |
| Name | Gene | of Edit | Base Editor | | NO | PAM |
| | HBG1/2 | c.−198 T > C | ABE | GUGGGGAAGGGGCCCC | 455 | AGG |
| | | | | CAAG | | |
| | HBG1/2 | c.−198 T > C | ABE | AUUGAGAUAGUGUGGG | 456 | GGG |
| | | | | GAAG | | |
| | HBG1/2 | c.−198 T > C | ABE | CAUUGAGAUAGUGUGG | 457 | GGG |
| | | | | GGAA | | |
| | HBG1/2 | c.−198 T > C | ABE | GCAUUGAGAUAGUGUG | 458 | AGG |
| | | | | GGGA | | |
| | HBG1/2 | c.−198 T > C | ABE | GUGGGGAAGGGGCCCC | 455 | AGG |
| | | | | CAAG | | |
| | HBG1/2 | c.−114~- | CBE and/or | GCUAUUGGUCAAGGCA | 459 | TGG |
| | | 102 deletion | ABE | AGGC | | |
| | HBG1/2 | c.−114~- | CBE and/or | CAAGGCUAUUGGUCAA | 460 | AGG |
| | | 102 deletion | ABE | GGCA | | |
| | HBG1/2 | c.−114~- | CBE and/or | CUUGUCAAGGCUAUUG | 461 | AGG |
| | | 102 deletion | ABE | GUCA | | |
| sgRNA_ | HBG1/2 | c.−114~- | CBE and/or | CUUGACCAAUAGCCUU | 462 | AGG |
| 015 | | 102 deletion | ABE | GACA | | |
| (TSBT | | | | | | |
| x045) | | | | | | |
| | HBG1/2 | c.−114~- | CBE and/or | GUUUGCCUUGUCAAGG | 463 | TGG |
| | | 102 deletion | ABE | CUAU | | |
| | HBG1/2 | c.−114~- | CBE and/or | UGGUCAAGUUUGCCUU | 464 | AGG |
| | | 102 deletion | ABE | GUCA | | |
| | HBG1/2 | c.−198 T > C | ABE | UGGGGAAGGGGCCCCC | 465 | GGA |
| | | | | AAGA | | |
| | HBG1/2 | c.−198 T > C | ABE | GUGUGGGGAAGGGGCC | 466 | AGA |
| | | | | CCCA | | |
| | HBG1/2 | c.−175 T > C | ABE | UCAGACAGAUAUUUGC | 467 | AGA |
| | | | | AUUG | | |
| | HBG1/2 | c.−175 T>C | ABE | UUUCAGACAGAUAUUU | 468 | TGA |
| | | | | GCAU | | |
| | HBG1/2 | c.−114~- | CBE and/or | CUUGCCUUGACCAAUA | 469 | TGA |
| | | 102 deletion | ABE | GCCU | | |
| | HBG1/2 | c.−114~- | CBE and/or | UAGCCUUGACAAGGCA | 470 | TGA |
| | | 102 deletion | ABE | AACU | | |
| | HBG1/2 | c.−90 | CBE and/or | CAAACUUGACCAAUAG | 471 | AGA |
| | | BCL11A | ABE | UCUU | | |
| | | binding | | | | |
| | HBG1/2 | c.−198 T > C | ABE | UGUGGGGAAGGGGCCC | 472 | GAGGA |
| | | | | CCAA | | T |
| | HBG1/2 | c.−202 | CBE and/or | GGGCCCCUUCCCCACA | 473 | CTCAA |
| | | C > T, −201 | ABE | CUAU | | T |
| | | C > T, −198 | | | | |
| | | T > C, −197 | | | | |
| | | C > T, −196 | | | | |
| | | C > T, −195 | | | | |
| | | C > G | | | | |
| | HBG1/2 | c.−175 T > C | ABE | CAGACAGAUAUUUGCA | 474 | GATAG |
| | | | | UUGA | | T |
| | HBG1/2 | c.−175 T > C | ABE | UUUCAGACAGAUAUUU | 468 | TGAGA |
| | | | | GCAU | | T |
| | HBG1/2 | c.−114~- | CBE and/or | GCCUUGACAAGGCAAA | 475 | ACCAA |
| | | 102 deletion | ABE | CUUG | | T |
| | HBG1/2 | c.−114~- | CBE and/or | UUGACAAGGCAAACUU | 476 | AATAG |
| | | 102 deletion | ABE | GACC | | T |
| | HBG1/2 | c.−90 | CBE and/or | UGACCAAUAGUCUUAG | 477 | TCCAG |
| | | BCL11A | ABE | AGUA | | T |
| | | binding | | | | |
| | HBG1/2 | c.−175 T > C | ABE | AGACAGAUAUUUGCAU | 478 | TTT |
| | | | | UGAGAUA | | |
| | HBG1/2 | | ABE and/or | AAGUUUGCCUUGUCAA | 479 | NNNRRT |
| | | | CBE | GGCU | | (AUUGGU) |

TABLE 2
Exemplary spacer sequences for editing a CD117 polypeptide

| | | SEQ | | SEQ | | |
|---|---|---|---|---|---|---|
| | | ID | | ID | | |
| Guide No. | Spacer | NO: | Target Sequence | NO: | PAM | Mutations |
| Guide-CC1 | AUGAAACGAAUG | 533 | ATGAAACGAATGAGA | 732 | CAG | E73G |
| | AGAAUAAG | | ATAAG | | | T74A |
| | | | | | | N75D |
| Guide-CC2 | UGGAUGAAACGA | 534 | TGGATGAAACGAATG | 733 | AAG | D72G |
| | AUGAGAAU | | AGAAT | | | E73G |
| | | | | | | T74A |
| Guide-CC3 | GCCAGGGGAAGC | 535 | GCCAGGGGAAGCCTC | 734 | AAG | Q152R |
| | CUCUUCCC | | TTCCC | | | K154E |
| Guide-CC4 | UCUGUGUACUCA | 536 | TCTGTGTACTCAACG | 735 | AAG | Y243C |
| | ACGUGGAA | | TGGAA | | | |
| Guide-CC5 | UGUGUACUCAAC | 537 | TGTGTACTCAACGTG | 736 | GAG | Y243C |
| | GUGGAAAA | | GAAAA | | | |
| Guide-CC6 | GGGAAGAUUAUC | 538 | GGGAAGATTATCCCA | 737 | GAG | E360G |
| | CCAAGUCU | | AGTCT | | | D361G |
| Guide-CC7 | CCAGAAAUCCUG | 539 | CCAGAAATCCTGACT | 738 | CAG | E414G |
| | ACUUACGA | | TACGA | | | I415V |
| Guide-CC8 | UCUGAGAAUGAA | 540 | TCTGAGAATGAAAGT | 739 | CAG | E366G |
| | AGUAAUAU | | AATAT | | | N367G |
| Guide-CC9 | CUAUUUUAACUU | 541 | CTATTTTAACTTTGC | 740 | AAG | Y503C |
| | UGCAUUUA | | ATTTA | | | N505G |
| Guide- | UUCACGAGCCUG | 542 | TTCACGAGCCTGTCG | 741 | CAG | L421P |
| CC10 | UCGUAAGU | | TAAGT | | | V422A |
| Guide- | CAGCAGUGGAUC | 543 | CAGCAGTGGATCTAT | 742 | CAG | Q347R |
| CC11 | UAUAUGAA | | ATGAA | | | |
| Guide- | CAUUCCCCAAAC | 544 | CATTCCCCAAACCTG | 743 | CAG | K342E |
| CC12 | CUGAACAC | | AACAC | | | |
| Guide- | UAUCCCAAGUCU | 545 | TATCCCAAGTCTGAG | 744 | AAG | K364G |
| CC13 | GAGAAUGA | | AATGA | | | |
| Guide- | AUAAAUGGGAAG | 546 | ATAAATGGGAAGATT | 745 | AAG | K358G |
| CC14 | AUUAUCCC | | ATCCC | | | |
| Guide- | UCCCCAAACCUG | 547 | TCCCCAAACCTGAAC | 746 | CAG | K342G |
| CC15 | AACACCAG | | ACCAG | | | |
| Guide- | GCCAUUCACGAG | 548 | GCCATTCACGAGCCT | 747 | AAG | V422A |
| CC16 | CCUGUCGU | | GTCGT | | | |
| Guide- | AAAUGUCACAAC | 549 | AAATGTCACAACAAC | 748 | AAG | N300S |
| CC17 | AACCUUGG | | CTTGG | | | T302A |
| Guide- | CGUGGAAAAGAG | 550 | CGTGGAAAAGAGAAA | 749 | CAG | K247G |
| CC18 | AAAACAGU | | ACAGT | | | R248G |
| Guide- | CCAUUUGACAAA | 551 | CCATTTGACAAAGCC | 750 | CAG | V64A |
| CC19 | GCCCGGAU | | CGGAT | | | W66R |
| Guide- | AGCAAGCUAUCU | 552 | AGCAAGCTATCTTCT | 751 | AAG | S220G |
| CC20 | UCUUAGGG | | TAGGG | | | Y221C |
| Guide- | UGAGGUUUAUUC | 553 | TGAGGTTTATTCCTG | 752 | AAG | R161G |
| CC21 | CUGACCCC | | ACCCC | | | I163V |
| Guide- | UGAAUGGAUCAC | 554 | TGAATGGATCACGGA | 753 | CAG | E81G |
| CC22 | GGAAAAGG | | AAAGG | | | I83V |
| Guide- | CCACUGGCAGUA | 555 | CCACTGGCAGTACAG | 754 | GAG | L455P |
| CC23 | CAGAAGCA | | AAGCA | | | V457A |
| Guide- | AGAAUGAAUGGA | 556 | AGAATGAATGGATCA | 755 | AAG | N80G |
| CC24 | UCACGGAA | | CGGAA | | | E81G |
| Guide- | UGUAAACGAUGG | 557 | TGTAAACGATGGAGA | 756 | TAG | N326G |
| CC25 | AGAAAAUG | | AAATG | | | D327G |
| Guide- | AGGCUUACAACG | 558 | AGGCTTACAACGATG | 757 | AAG | Y494C |
| CC26 | AUGUGGGC | | TGGGC | | | N495D |
| Guide- | AAGUGACCAAUU | 559 | AAGTGACCAATTATT | 758 | AAG | T144A |
| CC27 | AUUCCCUC | | CCCTC | | | N145D |
| Guide- | UGUCACAACAAC | 560 | TGTCACAACAACCTT | 759 | TAG | T302A |
| CC28 | CUUGGAAG | | GGAAG | | | T303A |
| Guide- | AAUAGAUUGGUA | 561 | AATAGATTGGTATTT | 760 | CAG | I438M |
| CC29 | UUUUUGUC | | TTGTC | | | D439G |
| Guide- | ACAAUGGCACGG | 562 | ACAATGGCACGGTTG | 761 | AAG | N486G |
| CC30 | UUGAAUGU | | AATGT | | | T488A |
| Guide- | ACACAACAGGCA | 563 | ACACAACAGGCACAG | 762 | AAG | V213A |
| CC31 | CAGCUUUG | | CTTTG | | | V214A |
| Guide- | GUUCAGAGUUCU | 564 | GTTCAGAGTTCTATA | 763 | TAG | Q475R |
| CC32 | AUAGAUUC | | GATTC | | | S476G |
| Guide- | AGAUUUGAUUGU | 565 | AGATTTGATTGTTGA | 764 | AAG | D332G |
| CC33 | UGAAUAUG | | ATATG | | | I334V |
| Guide- | AUAAACAUUAAA | 566 | ATAAACATTAAATGC | 765 | CAG | V407A |
| CC34 | UGCUAUGG | | TATGG | | | Y408H |
| Guide- | AUCCAUCCAUCC | 567 | ATCCATCCATCCAGG | 766 | CAG | I39V |
| CC35 | AGGAAAAU | | AAAAT | | | H40R |
| Guide- | AUGGAUCACGGA | 568 | ATGGATCACGGAAAA | 767 | AAG | I83V |
| CC36 | AAAGGCAG | | GGCAG | | | T84A |
| Guide- | GUUGACUAUCAG | 569 | GTTGACTATCAGTTC | 768 | GAG | T276A |
| CC37 | UUCAGCGA | | AGCGA | | | I277V |
| Guide- | CACAACAACCUU | 570 | CACAACAACCTTGGA | 769 | TAG | T303A |
| CC38 | GGAAGUAG | | AGTAG | | | T304A |
| Guide- | CUAUAGAUUCUA | 571 | CTATAGATTCTAGTG | 770 | AAG | I478V |
| CC39 | GUGCAUUC | | CATTC | | | D479G |
| Guide- | UGCCAAUAAUAC | 572 | TGCCAATAATACTTT | 771 | CAG | N293G |
| CC40 | UUUUGGAU | | TGGAT | | | N294G |
| Guide- | GCUUUGUCAAAU | 573 | GCTTTGTCAAATGGA | 772 | GAG | K65E |
| CC41 | GGACUUUU | | CTTTT | | | |
| Guide- | CUUCCCCAUGAU | 574 | CTTCCCCATGATAAA | 773 | CAG | M318V |
| CC42 | AAACACUA | | CACTA | | | |
| Guide- | CGUUUGGAAAGC | 575 | CGTTTGGAAAGCTAG | 774 | CAG | K471E |
| CC43 | UAGUGGUU | | TGGTT | | | |
| Guide- | AAACUACAGGAG | 576 | AAACTACAGGAGAAA | 775 | TAG | Q256R |
| CC44 | AAAUAUAA | | TATAA | | | |
| Guide- | AGGAGGCACUUA | 577 | AGGAGGCACTTACAC | 776 | TAG | T389A |
| CC45 | CACAUUCC | | ATTCC | | | |
| Guide- | CUUGAAUGCACU | 578 | CTTGAATGCACTAGA | 777 | TAG | F483P |
| CC46 | AGAAUCUA | | ATCTA | | | |
| Guide- | UUCCAAACGGUG | 579 | TTCCAAACGGTGGCC | 778 | GAG | F469P |
| CC47 | GCCCAGAU | | CAGAT | | | |
| Guide- | UCCAGGAAAAUC | 580 | TCCAGGAAAATCAGA | 779 | TAG | K43G |
| CC48 | AGACUUAA | | CTTAA | | | |
| Guide- | CCCUCAAGGGGU | 581 | CCCTCAAGGGGTGCC | 780 | AAG | K149G |
| CC49 | GCCAGGGG | | AGGGG | | | |
| Guide- | UCCAAAGCAAGC | 582 | TCCAAAGCAAGCTAT | 781 | TAG | K218G |
| CC50 | UAUCUUCU | | CTTCT | | | |
| Guide- | UAGGGAAGGGGA | 583 | TAGGGAAGGGGAAGA | 782 | CAG | E225G |
| CC51 | AGAAUUCA | | ATTCA | | | |
| Guide- | AGCGAGAGUUAA | 584 | AGCGAGAGTTAATGA | 783 | GAG | R281G |
| CC52 | UGAUUCUG | | TTCTG | | | |
| Guide- | UUUGGAAAGCUA | 585 | TTTGGAAAGCTAGTG | 784 | GAG | K471G |
| CC53 | GUGGUUCA | | GTTCA | | | |
| Guide- | AGACGGUUCCCC | 586 | AGACGGTTCCCCTGG | 785 | CAG | S35P |
| CC54 | UGGACUCA | | ACTCA | | | |
| Guide- | ACGUUGACUAUC | 587 | ACGTTGACTATCAGT | 786 | GAG | T276A |
| CC55 | AGUUCAGC | | TCAGC | | | |
| Guide- | CUGCACAUCCAC | 588 | CTGCACATCCACTGG | 787 | CAG | V459A |
| CC56 | UGGCAGUA | | CAGTA | | | |
| Guide- | CACAUCCACUGG | 589 | CACATCCACTGGCAG | 788 | AAG | V457A |
| CC57 | CAGUACAG | | TACAG | | | |
| Guide- | AUCCACUGGCAG | 590 | ATCCACTGGCAGTAC | 789 | CAG | V457A |
| CC58 | UACAGAAG | | AGAAG | | | |
| Guide- | UUGACGUCAGAA | 591 | TTGACGTCAGAATTG | 790 | TAG | V399A |
| CC59 | UUGGACAC | | GACAC | | | |
| Guide- | AUUAACUCUCGC | 592 | ATTAACTCTCGCTGA | 791 | TAG | V282A |
| CC60 | UGAACUGA | | ACTGA | | | |
| Guide- | CCAUACAAGGAG | 593 | CCATACAAGGAGCGG | 792 | AAG | L124P |
| CC61 | CGGUCAAC | | TCAAC | | | Y125H |
| Guide- | UUCAGGAUGAAU | 594 | TTCAGGATGAATTTT | 793 | CAG | I201T |
| CC62 | UUUUCCGA | | TCCGA | | | L202P |
| Guide- | GUGACUUCAAUU | 595 | GTGACTTCAATTATG | 794 | CAG | D266G |
| CC63 | AUGAACGU | | AACGT | | | N268D |
| Guide- | GUCGGAAAAAUU | 596 | GTCGGAAAAATTCAT | 795 | AAG | E198G |
| CC64 | CAUCCUGA | | CCTGA | | | K199G |
| Guide- | AUUAUAUUUCUC | 597 | ATTATATTTCTCCTG | 796 | TAG | Y259H |
| CC65 | CUGUAGUU | | TAGTT | | | |
| Guide- | AAGGAGCGGUCA | 598 | AAGGAGCGGTCAACA | 797 | AAG | S123P |
| CC66 | ACAAGGAA | | AGGAA | | | |
| Guide- | GAGAUUAAAAGG | 599 | GAGATTAAAAGGCAC | 798 | GAG | K383G |
| CC67 | CACCGAAG | | CGAAG | | | |
| Guide- | AACGAGAUUAAA | 600 | AACGAGATTAAAAGG | 799 | AAG | R381G |
| CC68 | AGGCACCG | | CACCG | | | |
| Guide- | GAAAAAUUCAUC | 601 | GAAAAATTCATCCTG | 800 | GAG | K199G |
| CC69 | CUGAAAGU | | AAAGT | | | |
| Guide- | AUUCAUCCUGAA | 602 | ATTCATCCTGAAAGT | 801 | CAG | I201V |
| CC70 | AGUGAGGC | | GAGGC | | | |
| Guide- | GAAAGAAGACAA | 603 | GAAAGAAGACAACGA | 802 | TGG | K127R |
| CC71 | CGACACGC | | CACGC | | | E128G |
| | | | | | | D129G |
| Guide- | UACAGGAGAAAU | 604 | TACAGGAGAAATATA | 803 | TGG | Q256R |
| CC72 | AUAAUAGC | | ATAGC | | | E257G |
| | | | | | | K258E |
| Guide- | AUGAGAAUAAGC | 605 | ATGAGAATAAGCAGA | 804 | TGG | E76G |
| CC73 | AGAAUGAA | | ATGAA | | | N77G |
| | | | | | | K78E |
| Guide- | CCAGGGGAAGCC | 606 | CCAGGGGAAGCCTCT | 805 | AGG | Q152R |
| CC74 | UCUUCCCA | | TCCCA | | | K154G |
| Guide- | CAGAAAUCCUGA | 607 | CAGAAATCCTGACTT | 806 | AGG | E414G |
| CC75 | CUUACGAC | | ACGAC | | | I415V |
| Guide- | ACAGAACCUUCA | 608 | ACAGAACCTTCACTG | 807 | TGG | R353G |
| CC76 | CUGAUAAA | | ATAAA | | | T354A |
| Guide- | UCACGAGCCUGU | 609 | TCACGAGCCTGTCGT | 808 | AGG | L421P |
| CC77 | CGUAAGUC | | AAGTC | | | V422A |
| Guide- | ACUUACGACAGG | 610 | ACTTACGACAGGCTC | 809 | TGG | Y418C |
| CC78 | CUCGUGAA | | GTGAA | | | D419G |
| Guide- | UGUUCAUAUAGA | 611 | TGTTCATATAGATCC | 810 | TGG | M351T |
| CC79 | UCCACUGC | | ACTGC | | | |
| Guide- | CCAAACCUGAAC | 612 | CCAAACCTGAACACC | 811 | TGG | K342G |
| CC80 | ACCAGCAG | | AGCAG | | | |
| Guide- | CUGUCGUAAGUC | 613 | CTGTCGTAAGTCAGG | 812 | TGG | Y418H |
| CC81 | AGGAUUUC | | ATTTC | | | |
| Guide- | CAGAACCUUCAC | 614 | CAGAACCTTCACTGA | 813 | GGG | T354A |
| CC82 | UGAUAAAU | | TAAAT | | | |
| Guide- | UAUUUUAACUUU | 615 | TATTTTAACTTTGCA | 814 | AGG | N505G |
| CC83 | GCAUUUAA | | TTTAA | | | |
| Guide- | UAAGCAGAAUGA | 616 | TAAGCAGAATGAATG | 815 | CGG | K78R |
| CC84 | AUGGAUCA | | GATCA | | | Q79R |
| | | | | | | N80D |
| Guide- | GAAUGGCAUGCU | 617 | GAATGGCATGCTCCA | 816 | TGG | N423S |
| CC85 | CCAAUGUG | | ATGTG | | | M425V |
| Guide- | GUGCAGACACUA | 618 | GTGCAGACACTAAAC | 817 | TGG | Q460R |
| CC86 | AACUCAUC | | TCATC | | | T461A |
| Guide- | UGACCAAUUAUU | 619 | TGACCAATTATTCCC | 818 | GGG | T144A |
| CC87 | CCCUCAAG | | TCAAG | | | N145G |
| Guide- | GUGACCAAUUAU | 620 | GTGACCAATTATTCC | 819 | GGG | T144A |
| CC88 | UCCCUCAA | | CTCAA | | | N145G |
| Guide- | AGUGACCAAUUA | 621 | AGTGACCAATTATTC | 820 | AGG | T144A |
| CC89 | UUCCCUCA | | CCTCA | | | N145G |
| Guide- | GAAUGAAUGGAU | 622 | GAATGAATGGATCAC | 821 | AGG | N80S |
| CC90 | CACGGAAA | | GGAAA | | | E81G |
| Guide- | UGCAGACACUAA | 623 | TGCAGACACTAAACT | 822 | GGG | Q460R |
| CC91 | ACUCAUCU | | CATCT | | | T461A |
| Guide- | CCAAAGCAAGCU | 624 | CCAAAGCAAGCTATC | 823 | AGG | K218G |
| CC92 | AUCUUCUU | | TTCTT | | | S220G |
| Guide- | ACAGCACUGACU | 625 | ACAGCACTGACTTGC | 824 | TGG | V195A |
| CC93 | UGCCCUCC | | CCTCC | | | L196P |
| Guide- | GGAGGGCAAGUC | 626 | GGAGGGCAAGTCAGT | 825 | CGG | E191G |
| CC94 | AGUGCUGU | | GCTGT | | | K193G |
| Guide- | CACAUAAACAUU | 627 | CACATAAACATTAAA | 826 | TGG | V407A |
| CC95 | AAAUGCUA | | TGCTA | | | Y408H |
| Guide- | CAAAUGGACUUU | 628 | CAAATGGACTTTTGA | 827 | TGG | K65R |
| CC96 | UGAGAUCC | | GATCC | | | T67A |
| Guide- | CAAUGGCACGGU | 629 | CAATGGCACGGTTGA | 828 | AGG | N486S |
| CC97 | UGAAUGUA | | ATGTA | | | T488A |
| Guide- | AAAUACUGUAGU | 630 | AAATACTGTAGTGTT | 829 | TGG | V323A |
| CC98 | GUUUAUCA | | TATCA | | | F324L |
| Guide- | CAAAGCAAGCUA | 631 | CAAAGCAAGCTATCT | 830 | GGG | K218R |
| CC99 | UCUUCUUA | | TCTTA | | | S220G |
| Guide- | GGAAUAAACCUC | 632 | GGAATAAACCTCAAG | 831 | GGG | F162P |
| CC100 | AAGUCCUU | | TCCTT | | | I163T |
| Guide- | ACCACUAGCUUU | 633 | ACCACTAGCTTTCCA | 832 | TGG | L472P |
| CC101 | CCAAACGG | | AACGG | | | V473A |
| Guide- | GCAAGCUAUCUU | 634 | GCAAGCTATCTTCTT | 833 | AGG | S220G |
| CC102 | CUUAGGGA | | AGGGA | | | Y221C |
| Guide- | CAAGCUAUCUUC | 635 | CAAGCTATCTTCTTA | 834 | GGG | S220G |
| CC103 | UUAGGGAA | | GGGAA | | | Y221C |
| Guide- | UUUGAUCAUGAU | 636 | TTTGATCATGATGCC | 835 | TGG | M171T |
| CC104 | GCCCGCCU | | CGCCT | | | I172T |
| Guide- | UUGAUCAUGAUG | 637 | TTGATCATGATGCCC | 836 | GGG | M171T |
| CC105 | CCCGCCUU | | GCCTT | | | I172T |
| Guide- | AUAGAUUGGUAU | 638 | ATAGATTGGTATTTT | 837 | AGG | I438M |
| CC106 | UUUUGUCC | | TGTCC | | | D439G |
| Guide- | AGGAAUAAACCU | 639 | AGGAATAAACCTCAA | 838 | TGG | F162P |
| CC107 | CAAGUCCU | | GTCCT | | | I163T |
| Guide- | CACAACAGGCAC | 640 | CACAACAGGCACAGC | 839 | AGG | V213A |
| CC108 | AGCUUUGA | | TTTGA | | | |
| Guide- | UGACGUCAGAAU | 641 | TGACGTCAGAATTGG | 840 | AGG | V399A |
| CC109 | UGGACACU | | ACACT | | | |
| Guide- | ACUACAGUAUUU | 642 | ACTACAGTATTTGTA | 841 | TGG | T322A |
| CC110 | GUAAACGA | | AACGA | | | |
| Guide- | AGCAAAUGUCAC | 643 | AGCAAATGTCACAAC | 842 | TGG | N300G |
| CC111 | AACAACCU | | AACCT | | | |
| Guide- | GUCUGAUUUUCC | 644 | GTCTGATTTTCCTGG | 843 | TGG | S44P |
| CC112 | UGGAUGGA | | ATGGA | | | |
| Guide- | UGCUGAUCCAAA | 645 | TGCTGATCCAAAAGT | 844 | TGG | S298P |
| CC113 | AGUAUUAU | | ATTAT | | | |
| Guide- | AUCAGACUUAAU | 646 | ATCAGACTTAATAGT | 845 | TGG | D45G |
| CC114 | AGUCCGCG | | CCGCG | | | |
| Guide- | UGCAUUCAAGCA | 647 | TGCATTCAAGCACAA | 846 | CGG | K484G |
| CC115 | CAAUGGCA | | TGGCA | | | |
| Guide- | UCCCAGAGCCCA | 648 | TCCCAGAGCCCACAA | 847 | TGG | E435G |
| CC116 | CAAUAGAU | | TAGAT | | | |
| Guide- | UCAGCGAGAGUU | 649 | TCAGCGAGAGTTAAT | 848 | TGG | R281G |
| CC117 | AAUGAUUC | | GATTC | | | |
| Guide- | GUGUGAAACGCG | 650 | GTGTGAAACGCGCCT | 849 | CGG | K176G |
| CC118 | CCUACCAU | | ACCAT | | | |
| Guide- | UCAGACUUAAUA | 651 | TCAGACTTAATAGTC | 850 | GGG | D45G |
| CC119 | GUCCGCGU | | CGCGT | | | |
| Guide- | UCUAGUGCAUUC | 652 | TCTAGTGCATTCAAG | 85 | TGG | S481G |
| CC120 | AAGCACAA | | CACAA | | | |
| Guide- | AUUCCCUCAAGG | 653 | ATTCCCTCAAGGGGT | 852 | GGG | K149E |
| CC121 | GGUGCCAG | | GCCAG | | | |
| Guide- | AAGGCAGAAGCC | 654 | AAGGCAGAAGCCACC | 853 | CGG | E88G |
| CC122 | ACCAACAC | | AACAC | | | |
| Guide- | GAGGUUUAUUCC | 655 | GAGGTTTATTCCTGA | 854 | AGG | I163V |
| CC123 | UGACCCCA | | CCCCA | | | |
| Guide- | UUUAUUCCUGAC | 656 | TTTATTCCTGACCCC | 855 | GGG | I163V |
| CC124 | CCCAAGGC | | AAGGC | | | |
| Guide- | GUUUAUUCCUGA | 657 | GTTTATTCCTGACCC | 856 | CGG | I163V |
| CC125 | CCCCAAGG | | CAAGG | | | |
| Guide- | CAUACAAGGAGC | 658 | CATACAAGGAGCGGT | 857 | AGG | L124P |
| CC126 | GGUCAACA | | CAACA | | | Y125H |
| Guide- | AAAAAUUCAUCC | 659 | AAAAATTCATCCTGA | 858 | AGG | K199G |
| CC127 | UGAAAGUG | | AAGTG | | | I201V |
| Guide- | AAAUAUAAUAGC | 660 | AAATATAATAGCTGG | 859 | CGG | Y259C |
| CC128 | UGGCAUCA | | CATCA | | | N260D/ |
| | | | | | | G/S |
| Guide- | UGACUUCAAUUA | 661 | TGACTTCAATTATGA | 860 | AGG | D266G |
| CC129 | UGAACGUC | | ACGTC | | | N268G |
| Guide- | CUAGUUCUGUGU | 662 | CTAGTTCTGTGTACT | 861 | TGG | S240G |
| CC130 | ACUCAACG | | CAACG | | | |
| Guide- | AGAUUAAAAGGC | 663 | AGATTAAAAGGCACC | 862 | AGG | K383G |
| CC131 | ACCGAAGG | | GAAGG | | | |
| Guide- | ACGAGAUUAAAA | 664 | ACGAGATTAAAAGGC | 863 | AGG | R381G |
| CC132 | GGCACCGA | | ACCGA | | | |
| Guide- | GGAUGAAACGAA | 665 | GGATGAAACGAATGA | 864 | AGC | E73G |
| CC133 | UGAGAAUA | | GAATA | | | T74A |
| | | | | | | N75G |
| Guide- | AACUACAGGAGA | 666 | AACTACAGGAGAAAT | 865 | AGC | Q256R |
| CC134 | AAUAUAAU | | ATAAT | | | E257G |
| | | | | | | K258G |
| Guide- | UGGGAAAGAAGA | 667 | TGGGAAAGAAGACAA | 866 | CGC | K127G |
| CC135 | CAACGACA | | CGACA | | | E128G |
| | | | | | | D129G |
| Guide- | GCCAAUAAUACU | 668 | GCCAATAATACTTTT | 867 | AGC | N293G |
| CC136 | UUUGGAUC | | GGATC | | | N294G |
| | | | | | | T295A |
| Guide- | CUUACGACAGGC | 669 | CTTACGACAGGCTCG | 868 | GGC | Y418C |
| CC137 | UCGUGAAU | | TGAAT | | | D419G |
| | | | | | | R420G |
| Guide- | GUUAAAAUAGGC | 670 | GTTAAAATAGGCAGA | 869 | TGC | Y503H |
| CC138 | AGAAGUCU | | AGTCT | | | F504P |
| Guide- | UUCUGUUCAUAU | 671 | TTCTGTTCATATAGA | 870 | TGC | Y350H |
| CC139 | AGAUCCAC | | TCCAC | | | M351T |
| Guide- | AGAAAUCCUGAC | 672 | AGAAATCCTGACTTA | 871 | GGC | I415V |
| CC140 | UUACGACA | | CGACA | | | T417A |
| Guide- | AACGAUGUGGGC | 673 | AACGATGTGGGCAAG | 872 | TGC | D496G |
| CC141 | AAGACUUC | | ACTTC | | | K499E |
| Guide- | AUUCCCCAAACC | 674 | ATTCCCCAAACCTGA | 873 | AGC | K342G |
| CC142 | UGAACACC | | ACACC | | | |
| Guide- | CGACAGGCUCGU | 675 | CGACAGGCTCGTGAA | 874 | TGC | R420G |
| CC143 | GAAUGGCA | | TGGCA | | | |
| Guide- | ACUUCUGCCUAU | 676 | ACTTCTGCCTATTTT | 875 | TGC | Y503C |
| CC144 | UUUAACUU | | AACTT | | | |
| Guide- | GCGACGAGAUUA | 677 | GCGACGAGATTAGGC | 876 | TGC | D52G |
| CC145 | GGCUGUUA | | TGTTA | | | E53G |
| | | | | | | I54V |
| | | | | | | R55G |
| Guide- | AAGACAACGACA | 678 | AAGACAACGACACGC | 877 | CGC | D129G |
| CC146 | CGCUGGUC | | TGGTC | | | N130G |
| | | | | | | D131G |
| | | | | | | T132A |
| Guide- | UGGAUCACGGAA | 679 | TGGATCACGGAAAAG | 878 | AGC | I83V |
| CC147 | AAGGCAGA | | GCAGA | | | T84A |
| | | | | | | E85G |
| | | | | | | K86E |
| Guide- | GACACAGACACA | 680 | GACACAGACACAACA | 879 | AGC | V213A |
| CC148 | ACAGGCAC | | GGCAC | | | V214A |
| | | | | | | S215P |
| | | | | | | V216A |
| Guide- | GGGAAGAAUUCA | 681 | GGGAAGAATTCACAG | 880 | TGC | E227G |
| CC149 | CAGUGACG | | TGACG | | | E228G |
| | | | | | | T230A |
| Guide- | GCACCAACAAAC | 682 | GCACCAACAAACACG | 881 | AGC | N99G |
| CC150 | ACGGCUUA | | GCTTA | | | K100G |
| | | | | | | H101R |
| Guide- | UAUAGAUUCUAG | 683 | TATAGATTCTAGTGC | 882 | AGC | I478M |
| CC151 | UGCAUUCA | | ATTCA | | | D479G |
| | | | | | | S481G |
| Guide- | UCAUGAUCAAAA | 684 | TCATGATCAAAAGTG | 883 | CGC | I172V |
| CC152 | GUGUGAAA | | TGAAA | | | K173G |
| | | | | | | S174G |
| Guide- | AUGAUCAAAAGU | 685 | ATGATCAAAAGTGTG | 884 | CGC | I172V |
| CC153 | GUGAAACG | | AAACG | | | K173G |
| | | | | | | S174G |
| Guide- | AAUGAAUGGAUC | 686 | AATGAATGGATCACG | 885 | GGC | E81G |
| CC154 | ACGGAAAA | | GAAAA | | | I83V |
| | | | | | | T84A |
| Guide- | CCAAUUAUUCCC | 687 | CCAATTATTCCCTCA | 886 | TGC | N145S |
| CC155 | UCAAGGGG | | AGGGG | | | Y146C |
| Guide- | CAGACUUAAUAG | 688 | CAGACTTAATAGTCC | 887 | GGC | D45G |
| CC156 | UCCGCGUG | | GCGTG | | | I47V |
| Guide- | UCUGACGUCAAU | 689 | TCTGACGTCAATGCT | 888 | AGC | D398G |
| CC157 | GCUGCCAU | | GCCAT | | | N400G |
| Guide- | GCAUAACAGCCU | 690 | GCATAACAGCCTAAT | 889 | CGC | L56P |
| CC158 | AAUCUCGU | | CTCGT | | | L57P |
| Guide- | UUUUUCCGACAG | 691 | TTTTTCCGACAGCAC | 890 | TGC | L196P |
| CC159 | CACUGACU | | TGACT | | | S197P |
| Guide- | UUUCCGUGAUCC | 692 | TTTCCGTGATCCATT | 891 | TGC | W82R |
| CC160 | AUUCAUUC | | CATTC | | | I83T |
| Guide- | UUUGACAAAGCC | 693 | TTTGACAAAGCCCGG | 892 | TGC | F63P |
| CC161 | CGGAUCAG | | ATCAG | | | V64A |
| Guide- | GCCCAGAUGAGU | 694 | GCCCAGATGAGTTTA | 893 | TGC | S464P |
| CC162 | UUAGUGUC | | GTGTC | | | S465P |
| Guide- | GCUGAACUGAUA | 695 | GCTGAACTGATAGTC | 894 | TGC | I277T |
| CC163 | GUCAACGU | | AACGT | | | S279P |
| Guide- | ACACUUUUGAUC | 696 | ACACTTTTGATCATG | 895 | CGC | M171T |
| CC164 | AUGAUGCC | | ATGCC | | | I172T |
| Guide- | GAAAAUCAGACU | 697 | GAAAATCAGACTTAA | 896 | CGC | K43R |
| CC165 | UAAUAGUC | | TAGTC | | | D45G |
| Guide- | GCAGACACUAAA | 698 | GCAGACACTAAACTC | 897 | GGC | T461A |
| CC166 | CUCAUCUG | | ATCTG | | | N463G |
| Guide- | GGACCAGGAGGG | 699 | GGACCAGGAGGGCAA | 898 | TGC | Q190R |
| CC167 | CAAGUCAG | | GTCAG | | | E191G |
| Guide- | GCUGAUCCAAAA | 700 | GCTGATCCAAAAGTA | 899 | GGC | F296P |
| CC168 | GUAUUAUU | | TTATT | | | S298P |
| Guide- | ACAUCCACUGGC | 701 | ACATCCACTGGCAGT | 900 | AGC | L455P |
| CC169 | AGUACAGA | | ACAGA | | | V457A |
| Guide- | CACUGGCAGUAC | 702 | CACTGGCAGTACAGA | 901 | AGC | V454A |
| CC170 | AGAAGCAG | | AGCAG | | | L455P |
| Guide- | AGGCAGAAGCCA | 703 | AGGCAGAAGCCACCA | 902 | GGC | E88G |
| CC171 | CCAACACC | | ACACC | | | T90A |
| Guide- | AGGUUUAUUCCU | 704 | AGGTTTATTCCTGAC | 903 | GGC | I163V |
| CC172 | GACCCCAA | | CCCAA | | | |
| Guide- | CCAAGCUUUUCC | 705 | CCAAGCTTTTCCTTG | 904 | CGC | K116R |
| CC173 | UUGUUGAC | | TTGAC | | | |
| Guide- | UUAGUGUCUGCA | 706 | TTAGTGTCTGCACAT | 905 | GGC | V459A |
| CC174 | CAUCCACU | | CCACT | | | |
| Guide- | CCACUAGCUUUC | 707 | CCACTAGCTTTCCAA | 906 | GGC | L472P |
| CC175 | CAAACGGU | | ACGGT | | | |
| Guide- | CCUCAAGGGGUG | 708 | CCTCAAGGGGTGCCA | 907 | AGC | K149G |
| CC176 | CCAGGGGA | | GGGGA | | | |
| Guide- | UGUGAAACGCGC | 709 | TGTGAAACGCGCCTA | 908 | GGC | K176G |
| CC177 | CUACCAUC | | CCATC | | | |
| Guide- | CUAGUGCAUUCA | 710 | CTAGTGCATTCAAGC | 909 | GGC | K484G |
| CC178 | AGCACAAU | | ACAAT | | | |
| Guide- | CCGCGUGGGCGA | 711 | CCGCGTGGGCGACGA | 910 | GGC | D52G |
| CC179 | CGAGAUUA | | GATTA | | | |
| Guide- | UUAUUCCUGACC | 712 | TTATTCCTGACCCCA | 911 | GGC | D165G |
| CC180 | CCAAGGCG | | AGGCG | | | |
| Guide- | ACUCCAGAAUCA | 713 | ACTCCAGAATCATTA | 912 | CGC | S285P |
| CC181 | UUAACUCU | | ACTCT | | | |
| Guide- | GGCUGUUAUGCA | 714 | GGCTGTTATGCACTG | 913 | GGC | T59A |
| CC182 | CUGAUCCG | | ATCCG | | | |
| Guide- | UUCAUCUAACGA | 715 | TTCATCTAACGAGAT | 914 | GGC | H378R |
| CC183 | GAUUAAAA | | TAAAA | | | T380A |
| | | | | | | R381G |
| Guide- | ACAGGAGAAAUA | 716 | ACAGGAGAAATATAA | 915 | GGC | E257G |
| CC184 | UAAUAGCU | | TAGCT | | | K258G |
| | | | | | | Y259C |
| Guide- | UCAGGAUGAAUU | 717 | TCAGGATGAATTTTT | 916 | AGC | F200P |
| CC185 | UUUCCGAC | | CCGAC | | | I201T |
| Guide- | GACUUCAAUUAU | 718 | GACTTCAATTATGAA | 917 | GGC | N268G |
| CC186 | GAACGUCA | | CGTCA | | | Y269C |
| Guide- | AGGAGCGGUCAA | 719 | AGGAGCGGTCAACAA | 918 | AGC | V120A |
| CC187 | CAAGGAAA | | GGAAA | | | S123P |
| Guide- | GAUUAAAAGGCA | 720 | GATTAAAAGGCACCG | 919 | GGC | K383G |
| CC188 | CCGAAGGA | | AAGGA | | | T385A |
| Guide- | AAAAUUCAUCCU | 721 | AAAATTCATCCTGAA | 920 | GGC | I201V |
| CC189 | GAAAGUGA | | AGTGA | | | |
| gRNA931; | AUAAUAGCUGGC | 722 | ATAATAGCTGGCATC | 921 | GACT | S261G |
| gRNA2342; | AUCACGGU | | ACGGT | | | N260D |
| gRNA2343; | | | | | | |
| CC200 | | | | | | |
| gRNA889 | CCACUAGCUUUC | 723 | CCACTAGCTTTCCAA | 922 | GGCC | L472P |
| | CAAACGGU | | ACGGT | | | |
| gRNA908 | GCUGAACUGAUA | 724 | GCTGAACTGATAGTC | 923 | TGCC | S279P |
| | GUCAACGU | | AACGT | | | |
| gRNA918 | UUUGACAAAGCC | 725 | TTTGACAAAGCCCGG | 924 | TGCA | V64A |
| | CGGAUCAG | | ATCAG | | | |
| gRNA923 | UGAAAGUGAGGC | 726 | TGAAAGTGAGGCCAG | 925 | GGCT | Silent |
| | CAGGUACU | | GTACT | | | |
| gRNA928 | AAACAGUCAGGU | 727 | AAACAGTCAGGTGAG | 926 | CGCT | S251G |
| | GAGUGAAU | | TGAAT | | | |
| gRNA929 | AACUACAGGAGA | 728 | AACTACAGGAGAAAT | 927 | AGCT | Silent |
| | AAUAUAAU | | ATAAT | | | |
| gRNA944 | GAUUAAAAGGCA | 729 | GATTAAAAGGCACCG | 928 | GGCA | Silent |
| | CCGAAGGA | | AAGGA | | | |
| gRNA291 | GGCUUUACCUCC | 730 | GGCTTTACCTCCAAT | 929 | AGG | A to G at |
| | AAUGGUGC | | GGTGC | | | splice site |
| | | | | | | (KO) |
| gRNA295 | CAGUACCUGGCC | 731 | CAGTACCTGGCCTCA | 930 | AGG | A to G at |
| | UCACUUUC | | CTTTC | | | splice site |
| | | | | | | (KO) |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof.

In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152(5):1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| spCas9-MQKFRAER | NGC |
| Cpf1 | 5′(TTTV) |
| SpyMac | 5′-NAA-3′ |

In some embodiments, the PAM is NGC. In some embodiments, the NGC PAM is recognized by a Cas9 variant. In some embodiments, the NGC PAM Cas9 variant includes one or more amino acid substitutions selected from D1135M, S1136Q, G1218K, E1219F, A1322R, D1332A, R1335E, and T1337R (collectively termed “MQKFRAER”) of spCas9 (SEQ ID NO: 197), or a corresponding mutation in another Cas9. In some embodiments, the Cas9 variant contains one or more amino acid substitutions selected from D1135V, G1218R, R1335Q, and T1337R (collectively termed VRQR) of spCas9 (SEQ ID NO: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID NO: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218). In some embodiments, the Cas9 variant includes one or more amino acid substitutions selected from D1135M, S1136Q, G1218K, E1219S, R1335E, and T1337R (collectively termed “MQKSER”) of spCas9 (SEQ ID No: 197), or a corresponding mutation in another Cas9. In some embodiments, the Cas9 variant includes one or more amino acid substitutions selected from D1135M, S1136Q, G1218K, E1219S, R1335E, and T1337R (collectively termed “MQKSER”) of spCas9 (SEQ ID NO: 197), or a corresponding mutation in another Cas9.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556(7699), 57-63; Miller et al., “Continuous evolution of SpCas9 variants compatible with non-G PAMs” Nat. Biotechnol., 2020 April; 38(4):471-481; the entire contents of each are hereby incorporated by reference.

### Fusion Proteins or Complexes Comprising a NapDNAbp and a Deaminase

Some aspects of the disclosure provide fusion proteins or complexes comprising a Cas9 domain or other nucleic acid programmable DNA binding protein (e.g., Cas12) and one or more adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the adenosine deaminases provided herein. The domains of the base editors disclosed herein can be arranged in any order.

In some embodiments, the fusion proteins or complexes comprising an adenosine deaminase and a napDNAbp (e.g., Cas9 or Cas12 domain) do not include a linker sequence. In some embodiments, a linker is present between the adenosine deaminase and the napDNAbp. In some embodiments, adenosine deaminase and the napDNAbp are fused via any of the linkers provided herein. For example, in some embodiments the adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA-Circular Permutant 136 ins1248 | ISLAY31 |
| IBE032 | TadA-Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)n (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | I | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
Adenosine Deaminase Variants. Alterations are referenced to
TadA*7.10. Additional details of TadA*9 adenosine deaminases
are described in International PCT Application No. PCT/US2020/
049975, which is incorporated herein by reference in its
entirety for all purposes.

| TadA*9 | |
|---|---|
| Description | Alterations |
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F_I76Y_V82S_Y123H_Y147R_Q154R |
| TadA*9.30 | I76Y_V82T_Y123H_Y147R_Q154R |
| TadA*9.31 | N38G_I76Y_V82S_Y123H_Y147R_Q154R |
| TadA*9.32 | N38G_I76Y_V82T_Y123H_Y147R_Q154R |
| TadA*9.33 | R23H_I76Y_V82S_Y123H_Y147R_Q154R |
| TadA*9.34 | P54C_I76Y_V82S_Y123H_Y147R_Q154R |
| TadA*9.35 | R21N_I76Y_V82S_Y123H_Y147R_Q154R |
| TadA*9.36 | I76Y_V82S_Y123H_D138M_Y147R_Q154R |
| TadA*9.37 | Y72S_I76Y_V82S_Y123H_Y147R_Q154R |
| TadA*9.38 | E25F_I76Y_V82S_Y123H_Y147R_Q154R |
| TadA*9.39 | I76Y_V82T_Y123H_Y147R_Q154R |
| TadA*9.40 | N38G_I76Y_V82S_Y123H_Y147R_Q154R |
| TadA*9.41 | N38G_I76Y_V82T_Y123H_Y147R_Q154R |
| TadA*9.42 | R23H_I76Y_V82S_Y123H_Y147R_Q154R |
| TadA*9.43 | P54C_I76Y_V82S_Y123H_Y147R_Q154R |
| TadA*9.44 | R21N_I76Y_V82S_Y123H_Y147R_Q154R |
| TadA*9.45 | I76Y_V82S_Y123H_D138M_Y147R_Q154R |
| TadA*9.46 | Y72S_I76Y_V82S_Y123H_Y147R_Q154R |
| TadA*9.47 | N72K V82S, Y123H, Y147R, Q154R |
| TadA*9.48 | Q71M_V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K V82S, Y123H, Y147R, Q154R |
| TadA*9.54 | Q71M_V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

In some embodiments, the adenosine deaminase comprises one or more of M1I, S2A, S2E, V4D, V4E, V4M, F6S, H8E, H8Y, E9Y, M12S, R13H, R13I, R13Y, T17L, T17S, L18A, L18E, A19N, R21N, K20K, K20R, R21A, G22P, W23D, R23H, W23G, W23Q, W23L, W23R, D24E, D24G, E25F, E25M, E25D, E25A, E25G, E25R, E25V, E25S, E25Y, R26D, R26E, R26G, R26N, R26Q, R26C, R26L, R26K, R26W, E27V, E27D, P29V, V30G, L34S, L34V, L36H, H36L, H36N, N37N, N37T, N37S, N38G, N38R, W45A, W45L, W45N, N46N, R46W, R46F, R46Q, R46M, R47A, R47Q, R47F, R47K, R47P, R47W, R47M, P48T, P48L, P48A, P48I, P48S, I49G, I49H, I49V, I49F, I49H, G50L, R51H, R51L, R51N, L51W, R51Y, H52D, H52Y, D53P, P54C, P54T, A55H, T55A, A56E, A56S, E59A, E59G, E591, E59Q, E59W, M61A, M61I, M61L, M61V, L63S, L63V, Q65V, G66C, G67D, G67L, G67V, L68Q, M70H, M70Q, L84F, M70V, M70L, E70A, M70V, Q71M, Q71N, Q71L, Q71R, N72A, N72K, N72S, N72D, N72Y, Y73G, Y73I, Y73K, Y73R, Y73S, R74A, R74Q, R74G, R74K, R74L, R74N, I76D, I76F, 1761, 176N, I76T, I76Y, D77G, A78I, T79M, L80M, L80Y, V82A, V82S, V82G, V82T, L84E, L84F, L84Y, E85K, E85G, E85P, E85S, S87C, S87L, S87V, V88A, V88M, C90S, A91A, A91G, A91S, A91V, A91T, G92T, A93I, M94A, M94V, M94L, M94I, M94H, I95S, I95G, I95L, I95H, I95V, H96A, H96L, H96R, H96S, S97C, S97G, S97I, S97M, S97R, S97S, R98K, R98I, R98N, R98Q, G100R, G100V, R101V, R101R, V102A, V102F, V1021, V102V, D103A, F104G, D104N, F104V, F1041, F104L, A106T, V106Q, V106F, V106W, V106M, A106A, A106Q, A106F, A106G, A106W, A106M, A106V, A106R, R107C, R107G, R107P, R107K, R107A, R107N, R107W, R107H, R107S, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, A109H, A109K, A109R, A109S, A109T, A109V, K110G, K110H, K110I, K110R, K110T, T111A, T111G, T111H, T111R, G112A, A114G, A114H, A114V, G115S, L117M, L117N, L117V, M118D, M118G, M118K, M118N, M118V, D119L, D119N, D119S, D119V, V120H, V120L, H122H, H122N, H122P, H122R, H122S, H122Y, H123C, H123G, H123P, H123V, H123Y, Y123H, P124G, P1241, P124L, P124W, G125H, G125I, G125A, G125M, G125K, M126D, M126H, M126K, M126I, M126N, M1260, M126S, M126Y, N127H, N127S, N127D, N127K, N127R, H128R, R129H, R129Q, R129V, R1291, R129E, R129V, 11321, 1132F, T133V, T133E, T133G, T133K, E134A, E134E, E134G, E1341, G135G, G135V, I136G, I136L, I136T, I137A, I137D, I137E, L137M, I137S, A138D, A138E, A138G, S138A, A138N, A138S, A138T, A138V, A138Y, D139E, D139I, D139C, D139L, D139M, E140A, E140C, E140L, E140R, A142N, A142D, A142G, A142A, A142L, A142S, A142T, A142N, A142S, A142V, A143D, A143E, A143G, A143D, A143G, A143E, A143L, A143W, A143M, A143S, A143Q, A143R, C146R, S146A, S146C, S146D, S146F, S146R, S146T, D147D, D147L, D147F, D147G, D147Y, Y147T, Y147R, Y147D, D147R, F148L, F148F, F148R, F148Y, F149C, F149M, F149R, F149Y, M151F, M151P, M151R, M151V, R152C, R152F, R152H, R152P, R152R, R153C, R153Q, R153R, R153V, Q154E, Q154H, Q154M, Q154R, Q154L, Q154S, Q154V, E155F, E155G, E1551, E155K, E155P, E155V, E155D, 1156A, 1156F, 1156D, 1156K, 1156N, 1156R, 1156Y, E157A, E157F, E1571, E157P, E157T, E157V, N157K, K157N, K157R, A158Q, A158K, A158V, Q159F, Q159K, Q159L, Q159N, K160A, K160S, K160E, K160K, K160N, K1611, K161A, K161N, K161Q, K161S, K161T, A162D, A162Q, R162H, R162P, A162S, Q163G, Q163H, Q163N, Q163R, S164I, S164R, S164Y, S165A, S165D, S165I, S165T, S165Y, T166D, T166K, T166I, T166N, T166P, T166R, D167S and/or D167N mutation in a TadA reference sequence (e.g., TadA*7.10,ecTadA, or TadA8e), and any alternative mutation at the corresponding position, or any substitution from R26, W23, E27, H36, R47, P48, R51, H52, R74, 176, V82, V88, M94, I95, H96, A106, D108, A109, K110, T111, A114, D119, H122, H123, M126, N127, A142, S146, D147, F149, R152, Q154, E155, 1156, E157, K161, T166, and/or D167, with respect to a TadA reference sequence, or a substitution of 2-50 amino acids in a TadA reference sequence, which may be selected from W23R, E27D, H36L, R47K, P48A, R51H, R51L, 176F, 176Y, V82S, A106V, D108G, A109S, K110R, T111H, A114V, D119N, H122R, H122N, H123Y, M126I, N127K, S146C, D147R, R152P, Q154R, E155V, 1156F, K157N, K161N, T166I, and D167N, or one or more corresponding mutations in another adenosine deaminase. Additional mutations are described in U.S. Patent Application Publication No. 2022/0307003 A1 and International Patent Application Publications No. WO 2023/288304 A2 and WO 2023/034959 A2, the disclosures of which are incorporated herein by reference in their entirety for all purposes.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA-7.10 | R | V | A | T | D | H | Y | F | T | D |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A•T to G•C in genomic DNA without DNA cleavage” Nature, 551, 464-471 (2017), the entire contents of which are hereby incorporated by reference.

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications (“heavy mods”). Such heavy mods can increase base editing ˜2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation.

In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite—2 parts, while monopartite NLSs are not). The NLS of nucleoplasmin, KR [PAATKKAGQA]KKKK (SEQ ID NO: 191), is the prototype of the ubiquitous bipartite signal: two clusters of basic amino acids, separated by a spacer of about 10 amino acids. The sequence of an exemplary bipartite NLS follows:

(SEQ ID NO: 328)
PKKKRKVEGADKRTADGSEFESPKKKRKV.

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein, for example ABE Variant A, ABE Variant B, ABE Variant C, ABE Variant D, ABE Variant E, ABE Variant F, ABE Variant G, ABE Variant H, ABE Variant I, ABE Variant J, ABE Variant K, or ABE Variant D comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U:G heteroduplex DNA can be responsible for a decrease in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be an adenine deaminase or an adenosine deaminase. In some embodiments, the adenosine base editor can deaminate adenine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a steril alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH2) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., adenosine deaminase), a uracil glycosylase inhibitor (UGI) domain, epitope tags, and reporter gene sequences.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 6 refers to a monomeric form of TadA*7.10 comprising the alterations described. The term “heterodimer” as used in Table 6 refers to the specified wild-type E. coli TadA adenosine deaminase fused to a TadA*7.10 comprising the alterations as described.

TABLE 6
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + |
| | | Q154S |
| ABE-680m | MSP680 | monomer_TadA*7.10 + I76Y + V82G + |
| | | Y147T + Q154S |
| ABE-823m | MSP823 | monomer_TadA*7.10 + L36H + V82G + |
| | | Y147T + Q154S + N157K |
| ABE-824m | MSP824 | monomer_TadA*7.10 + V82G + Y147D + |
| | | F149Y + Q154S + D167N |
| ABE-825m | MSP825 | monomer_TadA*7.10 + L36H + V82G + |
| | | Y147D + F149Y + Q154S + N157K + D167N |
| ABE-827m | MSP827 | monomer_TadA*7.10 + L36H + I76Y + |
| | | V82G + Y147T + Q154S + N157K |
| ABE-828m | MSP828 | monomer TadA*7.10 + I76Y + V82G + |
| | | Y147D + F149Y + Q154S + D167N |
| ABE-829m | MSP829 | monomer_TadA*7.10 + L36H + I76Y + |
| | | V82G + Y147D + F149Y + Q154S + |
| | | N157K + D167N |
| ABE-605d | MSP605 | heterodimer_(WT) + (TadA*7.10 + V82G + |
| | | Y147T + Q154S) |
| ABE-680d | MSP680 | heterodimer_(WT) + (TadA*7.10 + 176Y + |
| | | V82G + Y147T + Q154S) |
| ABE-823d | MSP823 | heterodimer_(WT) + (TadA*7.10 + L36H + |
| | | V82G + Y147T + Q154S + N157K) |
| ABE-824d | MSP824 | heterodimer (WT) + (TadA*7.10 + V82G + |
| | | Y147D + F149Y + Q154S + D167N) |
| ABE-825d | MSP825 | heterodimer_(WT) + (TadA*7.10 + L36H + |
| | | V82G + Y147D + F149Y + Q154S + |
| | | N157K + D167N) |
| ABE-827d | MSP827 | heterodimer_(WT) + (TadA*7.10 + L36H + |
| | | I76Y + V82G + Y147T + Q154S + N157K) |
| ABE-828d | MSP828 | heterodimer_(WT) + (TadA*7.10 + I76Y + |
| | | V82G + Y147D + F149Y + Q154S + D167N) |
| ABE-829d | MSP829 | heterodimer_(WT) + (TadA*7.10 + L36H + |
| | | I76Y + V82G + Y147D + F149Y + Q154S + |
| | | N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise an adenosine deaminase and a Cas9 domain that are fused to each other via a linker. Various linker lengths and flexibilities between the adenosine deaminase and the Cas9 domain can be employed (e.g., ranging from very flexible linkers of the form (GGGS)ₙ (SEQ ID NO: 246), (GGGGS)ₙ (SEQ ID NO: 247), and (G)ₙ to more rigid linkers of the form (EAAAK)ₙ (SEQ ID NO: 248), (SGGS)ₙ (SEQ ID NO: 355), SGSETPGTSESATPES (SEQ ID NO: 249) (see, e.g. Guilinger J P, et al. Fusion of catalytically inactive Cas9 to FokI nuclease improves the specificity of genome modification. Nat. Biotechnol. 2014; 32(6): 577-82; the entire contents are incorporated herein by reference) and (XP)n) in order to achieve the optimal length for activity for the adenosine deaminase nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, an adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of

(SEQ ID NO: 356)
SGGSSGSETPGTSESATPESSGGS,
(SEQ ID NO: 357)
SGGSSGGSSGSETPGTSESATPESSGGSSGGS,
or
(SEQ ID NO: 358)
GGSGGSPGSPAGSPTSTEEGTSESATPESGPGTSTEPSEGSAPGSPAGS
PTSTEEGTSTEPSEGSAPGTSTEPSEGSAPGTSESATPESGPGSEPATS
GGSGGS.

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSG GS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTSTEE
GTSTEPSEGSAPGTSTEPSEGSAPGTSESATPESGPGSEPATS.

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder).

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising an Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, an adenosine deaminase nucleobase editor described herein is capable of making multiple mutations within a target sequence. These mutations may affect the function of the target. For example, when an adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain) can be used to edit a nucleotide from A to G.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A•T to G•C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Fusion proteins or complexes of the disclosure comprising a deaminase may be expressed in virtually any host cell of interest, including but not limited to bacteria, yeast, fungi, insects, plants, and animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A polynucleotide encoding a polypeptide described herein can be obtained by chemically synthesizing the polynucleotide, or by connecting synthesized partly overlapping oligo short chains by utilizing the PCR method and the Gibson Assembly method to construct a polynucleotide (e.g., DNA) encoding the full length thereof. The advantage of constructing a full-length polynucleotide by chemical synthesis or a combination of PCR method or Gibson Assembly method is that the codons to be used can be selected in according to the host into which the polynucleotide is to be introduced. In the expression from a heterologous DNA molecule, the protein expression level is expected to increase by converting the DNA sequence thereof to a codon highly frequently used in the host organism. Codon use data for a host cell (e.g., codon use data available at kazusa.or.jp/codon/index.html) can be used to guide codon optimization for a polynucleotide sequence encoding a polypeptide. Codons having low use frequency in the host may be converted to a codon coding the same amino acid and having high use frequency.

An expression vector containing a polynucleotide encoding a nucleic acid sequence-recognizing module and/or a nucleic acid base converting enzyme can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as .lambda phage and the like; insect virus vectors such as baculovirus and the like (e.g., BmNPV, AcNPV); animal virus vectors such as retrovirus, vaccinia virus, adenovirus and the like, and the like are used.

Regarding the promoter to be used, any promoter appropriate for a host to be used for gene expression can be used. In a conventional method using double-stranded breaks, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like may be used.

When the host is Escherichia coli, a trp promoter, lac promoter, recA promoter, .lamda.P.sub.L promoter, lpp promoter, T7 promoter, and the like can be used.

When the host is in the genus Bacillus, the SPO1 promoter, SPO2 promoter, penP promoter, and the like can be used.

When the host is a yeast, the Gal1/10 promoter, PHO5 promoter, PGK promoter, GAP promoter, ADH promoter, and the like can be used.

When the host is an insect cell, the polyhedrin promoter, P10 promoter, and the like can be used.

When the host is a plant cell, the CaMV35S promoter, CaMV19S promoter, NOS promoter, and the like can be used.

Expression vectors for use in the present disclosure, besides those mentioned above, can comprise an enhancer, a splicing signal, a terminator, a polyA addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the disclosure can be intracellularly expressed by introducing into the cell an expression vector comprising a nucleic acid sequence encoding the fusion protein or complex.

Host cells of interest, include but are not limited to bacteria, yeast, fungi, insects, plants, and animal cells. For example, a host cell may comprise bacteria from the genus Escherichia, such as Escherichia coli K12.cndot.DH1 [Proc. Natl. Acad. Sci. USA, 60, 160 (1968)], Escherichia coli JM103 [Nucleic Acids Research, 9, 309 (1981)], Escherichia coli JA221 [Journal of Molecular Biology, 120, 517 (1978)], Escherichia coli HB101 [Journal of Molecular Biology, 41, 459 (1969)], Escherichia coli C600 [Genetics, 39, 440 (1954)] and the like.

A host cell may comprise bacteria from the genus Bacillus, for example Bacillus subtilis M1114 [Gene, 24, 255 (1983)], Bacillus subtilis 207-21 [Journal of Biochemistry, 95, 87 (1984)] and the like.

A host cell may be a yeast cell. Examples of yeast cells include Saccharomyces cerevisiae AH22, AH22R.sup.−, NA87-11A, DKD-5D, 20B-12, Schizosaccharomyces pombe NCYC1913, NCYC2036, Pichia pastoris KM71 and the like.

When the viral delivery methods utilize the virus AcNPV, cells from a cabbage armyworm larva-derived established line (Spodoptera frugiperda cell; Sf cell), MG1 cells derived from the mid-intestine of Trichoplusia ni, High Five™ cells derived from an ovary of Trichoplusia ni, Mamestra brassicae-derived cells, Estigmena acrea-derived cells and the like can be used. When the virus is BmNPV, cells of Bombyx mori-derived established line (Bombyx mori N cell; BmN cell) and the like are used. As the Sf cell, for example, Sf9 cell (ATCC CRL1711), Sf21 cell [all above, In Vivo, 13, 213-217 (1977)] and the like are used.

An insect can be any insect, for example, larva of Bombyx mori, Drosophila, cricket, and the like [Nature, 315, 592 (1985)].

Animal cells contemplated in the present disclosure include, but are not limited to, cell lines such as monkey COS-7 cells, monkey Vero cells, Chinese hamster ovary (CHO) cells, dhfr gene-deficient CHO cells, mouse L cells, mouse AtT-20 cells, mouse myeloma cells, rat GH3 cells, human FL cells and the like, pluripotent stem cells such as iPS cells, ES cells derived humans and other mammals, and primary cultured cells prepared from various tissues. Furthermore, zebrafish embryo, Xenopus oocyte, and the like can also be used.

Plant cells are also contemplated in the present disclosure. Plant cells include, but are not limited to, suspended cultured cells, callus, protoplast, leaf segment, root segment and the like prepared from various plants (e.g., grain such as rice, wheat, corn, and the like; product crops such as tomato, cucumber, eggplant and the like; garden plants such as carnations, Eustoma russellianum, and the like; and other plants such as tobacco, Arabidopsis thaliana and the like) are used.

All the above-mentioned host cells may be haploid (monoploid), or polyploid (e.g., diploid, triploid, tetraploid, etc.). Using conventional methods, mutations, in principle, introduced into only one homologous chromosome produce a heterogenous cell. Therefore, the desired phenotype is not expressed unless the mutation is dominant. For recessive mutations, acquiring a homozygous cell can be inconvenient due to labor and time requirements. In contrast, according to the present disclosure, since a mutation can be introduced into any allele on the homologous chromosome in the genome, the desired phenotype can be expressed in a single generation even in the case of recessive mutation, thereby solving the problem associated with conventional mutagenesis methods.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

Escherichia coli can be transformed according to the methods described in, for example, Proc. Natl. Acad. Sci. USA, 69, 2110 (1972), Gene, 17, 107 (1982).

The genus Bacillus can be introduced into a vector according to the methods described in, for example, Molecular & General Genetics, 168, 111 (1979).

A yeast can be introduced into a vector according to the methods described in, for example, Methods in Enzymology, 194, 182-187 (1991), Proc. Natl. Acad. Sci. USA, 75, 1929 (1978).

An insect cell and an insect can be introduced into a vector according to the methods described in, for example, Bio/Technology, 6, 47-55 (1988).

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host. For example, when Escherichia coli or genus Bacillus is cultured, a liquid medium may be used as a medium to be used for the culture. The medium may contain a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is between about 5 about 8 in embodiments.

As a medium for culturing Escherichia coli, for example, M9 medium containing glucose, casamino acid [Journal of Experiments in Molecular Genetics, 431-433, Cold Spring Harbor Laboratory, New York 1972] can be used. Where necessary, for example, agents such as 30-indolylacrylic acid may be added to the medium to ensure an efficient function of a promoter. Escherichia coli is cultured at generally about 15 to about 43° C. Where necessary, aeration and stirring may be performed.

The genus Bacillus is cultured at generally about 30 to about 40° C. Where necessary, aeration and stirring may be performed.

Examples of the medium for culturing yeast include Burkholder minimum medium [Proc. Natl. Acad. Sci. USA, 77, 4505 (1980)], SD medium containing 0.5% casamino acid [Proc. Natl. Acad. Sci. USA, 81, 5330 (1984)] and the like. The pH of the medium may be between about 5 to about 8. The culture is performed at generally about 20° C. to about 35° C. Where necessary, aeration and stirring may be performed.

As a medium for culturing an insect cell or insect, for example, Grace's Insect Medium [Nature, 195, 788 (1962)] containing an additive such as inactivated 10% bovine serum and the like as appropriate and the like are used. The pH of the medium is may be between about 6.2 to about 6.4. The culture is performed at generally about 27° C. Where necessary, aeration and stirring may be performed.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium may be between about 6 to about 8. The culture is performed at generally about 30° C. to about 40° C. Where necessary, aeration and stirring may be performed.

As a medium for culturing a plant cell, for example, MS medium, LS medium, B5 medium and the like are used. The pH of the medium may be between about 5-about 8. The culture is performed at generally about 20° C. to about 30° C. Where necessary, aeration and stirring may be performed.

When a higher eukaryotic cell, such as animal cell, insect cell, plant cell and the like is used as a host cell, a polynucleotide encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Prokaryotic cells such as Escherichia coli and the like can utilize an inducible promoter. Examples of the inducible promoter include, but are not limited to, lac promoter (induced by IPTG), cspA promoter (induced by cold shock), araBAD promoter (induced by arabinose) and the like.

Alternatively, the above-mentioned inductive promoter can also be utilized as a vector removal mechanism when higher eukaryotic cells, such as animal cell, insect cell, plant cell and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient HDR with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (QssDNA) donors.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publication No. WO 2013/045632, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminus of each fragment is fused to an intein-N and the C-terminus of each fragment is fused to an intein C at amino acid positions 5303, T310, T313, S355, A456, S460, A463, T466, S469, T472, T474, C574, S577, A589, and S590, referenced to SEQ ID NO: 197.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof one or more pharmaceutical compositions comprising one or more cells having at least one edited gene. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of a disease or disorder in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Adenosine Base Editors with Increased Editing Efficiency

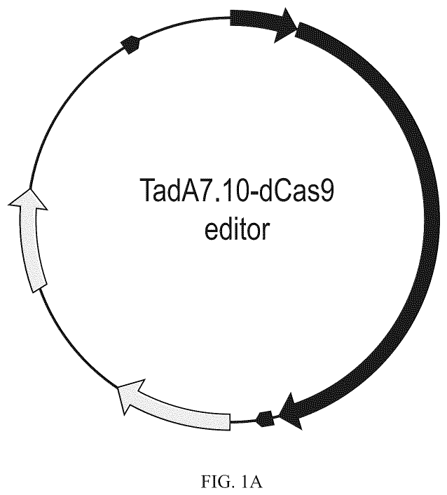

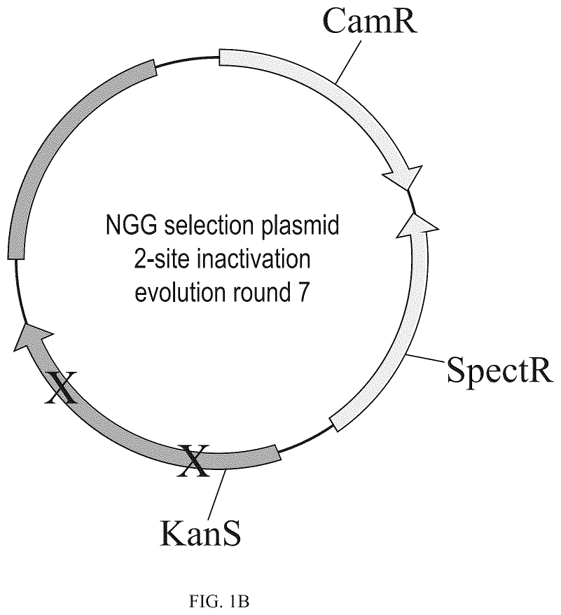

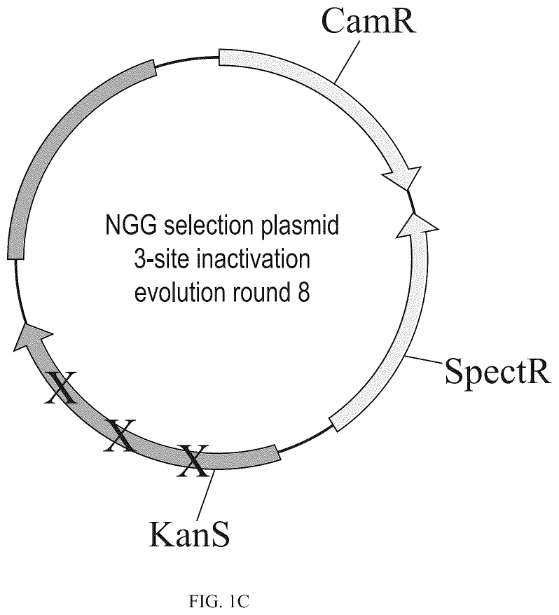

Base editing systems that include a Tad7.10-dCas9 fusion proteins are capable of editing a target polynucleotide with approximately 10-20% efficiency, but for uses requiring higher efficiency their use may be limited. In an effort to identify adenine base editors having increased efficiency and specificity, constructs comprising the adenosine deaminase TadA 7.10 were mutagenized by error prone PCR and subsequently cloned into an expression vector adjacent to a nucleic acid sequence encoding dCas9, a nucleic acid programmable DNA binding protein (FIG. 1A). The expression vectors comprising the adenosine deaminase variants were co-transformed into competent bacterial cells with a selection plasmid encoding chloramphenicol resistance (CamR) and spectinomycin resistance (SpectR) and having a kanamycin resistance gene that was rendered nonfunctional by two point mutations (evolution round 7 strategy) (FIG. 1B). The cells were selected for restoration of kanamycin resistance, which was a read out for adenosine deaminase activity. In subsequent rounds of selection, the expression vectors were co-transformed into competent cells with a plasmid encoding chloramphenicol resistance (CamR) and spectinomycin resistance (SpectR) and having a kanamycin resistance gene that was rendered nonfunctional by three point mutations (evolution round 8 strategy) (FIG. 1C).

An inactivated kanamycin resistance gene nucleic acid sequence is provided below:

(SEQ ID NO: 480)
ccggaattgccagctggggcgccctctggtaaggttgggaagccctgca
aagtaaactggatggctttcttgccgccaaggatctgatggcgcagggg
atcaagatctgatcaagagacaggatgaggatcctttcgcATGATCGAA
TAAGATGGATTGCACGCAGGTTCTCCGGCCGCTTAGGTGGAGCGCCTAT
TCGGCTATGACTGGGCACAACAGACAATCGGCTGCTCTGATGCCGCCGT
GTTCCGGCTGTCAGCGCAGGGGCGCCCGGTTCTTTTTGTCAAGACCGAC
CTGTCCGGTGCCCTGAATGAACTGCAGGACGAGGCAGCGCGGCTATCGT
GGCTGGCCACGACGGGCGTTCCTTGCGCAGCTGTGCTCGACGTTGTCAC
TGAAGCGGGAAGGGACTGGCTGCTATTGGGCGAAGTGCCGGGGCAGGAT
CTCCTGTCATCTCACCTTGCTCCTGCCGAGAAAGTATCCATCATGGCTG
ATGCAATGCGGCGGCTGCATACGCTTGATCCGGCTACCTGCCCATTCGA
CCACCAAGCGAAACATCGCATCGAGCGAGCACGTACTCGGATGGAAGCC
GGTCTTGTCGATCAGGATGATCTGGACGAAGAGCATCAGGGGCTCGCGC
CAGCCGAACTGTTCGCCAGGCTCAAGGCGCGCATGCCCGACGGCGAGGA
TCTCGTCGTGACCCATGGCGATGCCTGCTTGCCGAATATCATGGTGGAA
AATGGCCGCTTTTCTGGATTCATTAACTGTGGCCGGCTGGGTGTGGCGG
ACCGCTATCAGGACATAGCGTTGGCTACCCGTGATATTGCTGAAGAGCT
TGGCGGCGAATGGGCTGACCGCTTCCTCGTGCTTTACGGTATCGCCGCT
CCCGATTCGCAGCGCATCGCCTTCTATCGCCTTCTTGACGAGTTCTTCT
AA

In the above sequence, lower case denotes the kanamycin resistance promoter region, bold sequence indicates targeted inactivation portion (Q4* and W15*), the italicized sequence denotes the targeted inactive site of kanamycin resistance gene (D208N), and the underlined sequences denote the PAM sequences.

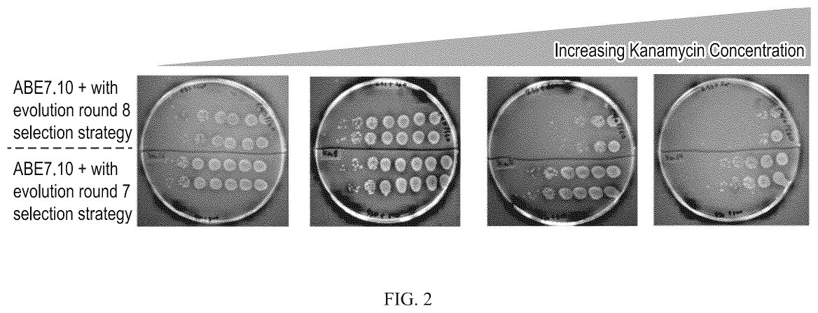

Again, the cells were plated onto a series of agarose plates with increasing kanamycin concentration. As shown in FIG. 2, adenosine deaminase variants having efficient base editing activity were able to correct the mutations present in the kanamycin resistance gene and were selected for further analysis. Adenosine deaminase variants used in base editors showing efficient base editing in bacterial cells are described in Table 5B. Mammalian expression vectors encoding base editors comprising the selected adenosine deaminase variants were generated.

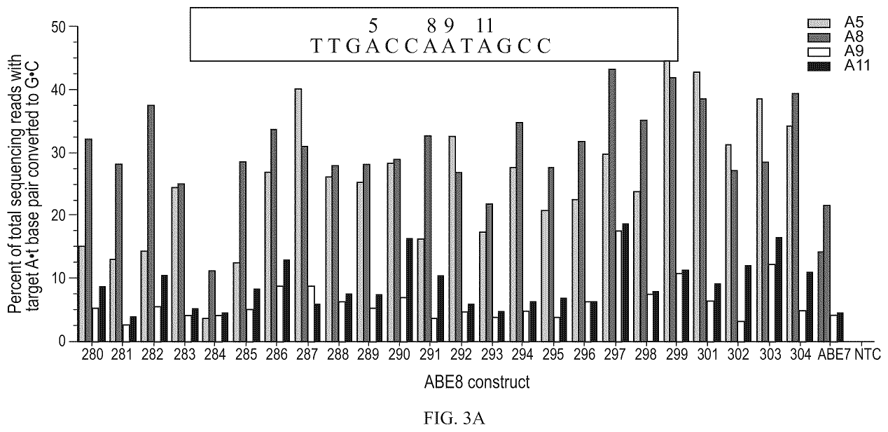

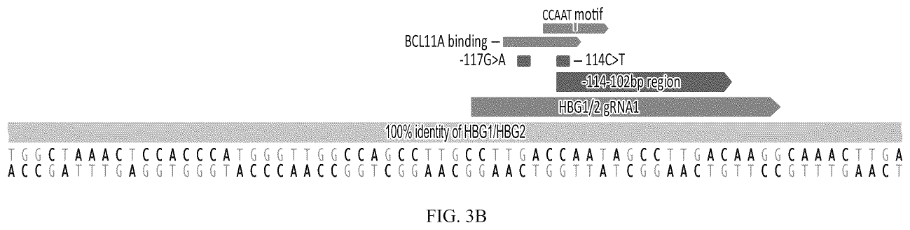

Hek293T cells expressing a β-globin protein associated with sickle cell disease that contained an E6V (also termed E7V) mutation were used to test the editing efficiency of the adenosine deaminase variants (FIGS. 3A and 3B). These cells termed “Hek293T/HBBE6V” cells were transduced using lentiviral vectors expressing a base editing system that included a fusion protein comprising the ABE8s listed in Table 7. The ABE8s were generated by cloning an adenosine deaminase variant into a scaffold that included a circular permutant Cas9 and a bipartite nuclear localization sequence. Circular permutant Cas9s are known in the art and described, for example, in Oakes et al., Cell 176, 254-267, 2019. These sequences are provided herein below.

Upregulation of fetal hemoglobin is a therapeutic approach to overcoming sickle cell disease. FIG. 3A shows a therapeutically relevant site for upregulation of fetal hemoglobin. Editing adenosines at residues 5 and 8 can significantly reduce BCL11A binding, thereby increasing expression of fetal hemoglobin. Referring to FIG. 3A, the ABE8s exhibited approximately 2-3 fold more base editing activity than the base editor ABE7.10.

TABLE 7
Novel Adenine Base Editors ABE8

| plasmid ID | description |
|---|---|
| 280 | ABE8.1 |
| 281 | ABE8.2 |
| 282 | ABE8.3 |
| 283 | ABE8.4 |
| 284 | ABE8.5 |
| 285 | ABE8.6 |
| 286 | ABE8.7 |
| 287 | ABE8.8 |
| 288 | ABE8.9 |
| 289 | ABE8.10 |
| 290 | ABE8.11 |
| 291 | ABE8.12 |
| 292 | ABE8.13 |
| 293 | ABE8.14 |
| 294 | ABE8.15 |
| 295 | ABE8.16 |
| 296 | ABE8.17 |
| 297 | ABE8.18 |
| 298 | ABE8.19 |
| 299 | ABE8.20 |
| 300 | ABE8.21 |
| 301 | ABE8.22 |
| 302 | ABE8.23 |
| 303 | ABE8.24 |
| 304 | ABE8.25 |

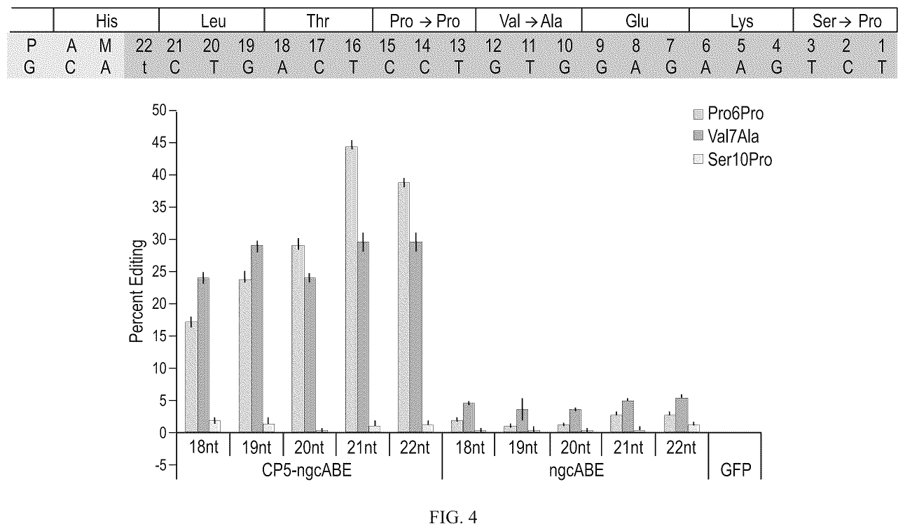

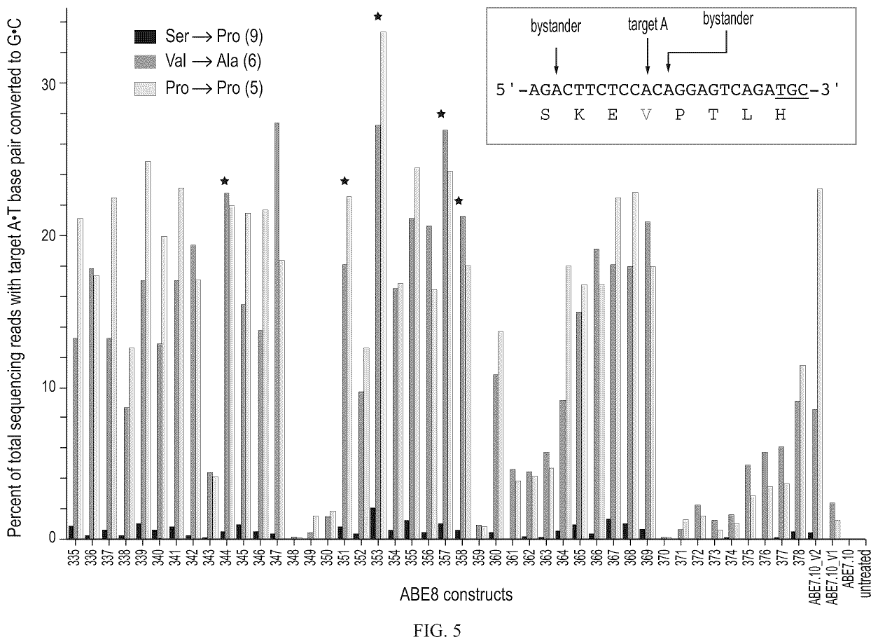

Referring to FIG. 4, the ABE8s were introduced into Hek293T/HBBE6V cells along with 18, 19, 20, 21, or 22 nucleotide guide RNAs targeting the polynucleotide encoding HBB E6V. The ABE8 editors showed increased editing efficiency when fused to circular permutant (Cp)-Cas9. In total, 40 different ABE8 constructs (Table 8) and three ABE7.10 constructs were tested for editing activity in Hek293T/HBBE6V cells. The sequence of exemplary constructs follows. To evaluate the specificity of editing, target and unintended or bystander mutations were monitored (FIG. 5). Unintended editing of an adenosine in codon 5 was silent. However, unintended editing of codon 9 resulted in a serine to proline mutation. Referring again to FIG. 5, multiple ABE8s showed increased editing efficiency and specificity compared to the ABE7.10 editors, and none of the editors had significant bystander editing that led to the serine to proline missense mutation.

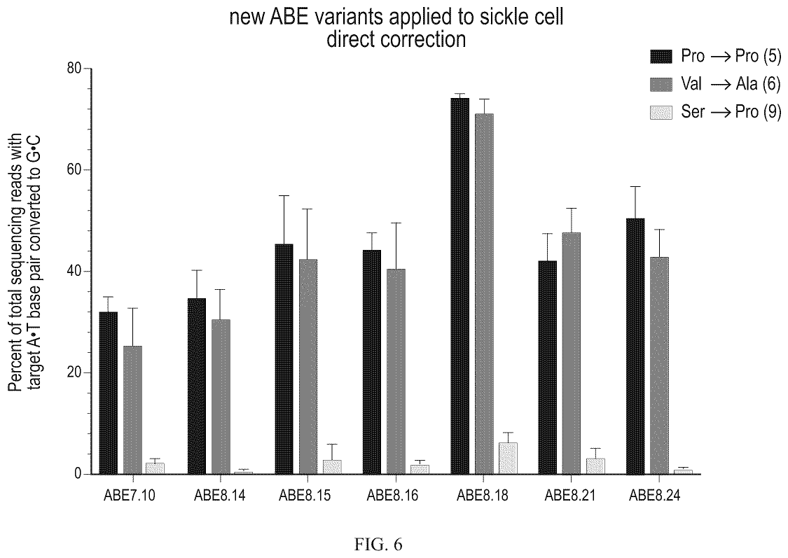

Further analysis of selected ABE8s and an ABE7.10 control was carried out in fibroblast cells containing the sickle cell mutation. As shown in FIG. 6, the ABE8 editors had increased base editing activity compared to the ABE7.10. ABE8.18 showed approximately 70% efficiency. The selected ABE8 editors also displayed unprecedented specificity. Importantly, the average INDEL formation for all ABE8 editors was less than 0.1%.

TABLE 8
Base editor constructs.

| plasmid | | |
|---|---|---|
| ID | description | function |
| 335 | NGC PAM CP5 variant (S. | monomer_TadA*7.10 + Y147T |
| | pyogenes Cas9)_ABE8.1 | |
| 336 | NGC PAM CP5 variant (S. | monomer_TadA*7.10 + Y147R |
| | pyogrouns Cas9)_ABE8.2 | |
| 337 | NGC PAM CP5 variant (S. | monomer_TadA*7.10 + Q154S |
| | pyogenes Cas9)_ABE8.3 | |
| 338 | NGC PAM CP5 variant (S. | monomer_TadA*7.10 + Y123H |
| | pyogenes Cas9)_ABE8.4 | |
| 339 | NGC PAM CP5 variant (S. | monomer_TadA*7.10 + V82S |
| | pyogenes Cas9)_ABE8.5 | |
| 340 | NGC PAM CP5 variant (S. | monomer_TadA*7.10 + T166R |
| | pyogenes Cas9)_ABE8.6 | |
| 341 | NGC PAM CP5 variant (S. | monomer_TadA*7.10 + Q154R |
| | pyogenes Cas9)_ABE8.7 | |
| 342 | NGC PAM CP5 variant (S. | monomer_Y147R_Q154R_ |
| | pyogenesCas9)_ABE8.8 | Y123H |
| 343 | NGC PAM CP5 variant (S. | monomer_Y147R_Q154R_I76Y |
| | pyogenes Cas9)_ABE8.9 | |
| 344 | NGC PAM CP5 variant (S. | monomer_Y147R_Q154R_ |
| | pyogenes Cas9)_ABE8.10 | T166R |
| 345 | NGC PAM CP5 variant (S. | monomer_Y147T_Q154R |
| | pyogenes Cas9)_ABE8.11 | |
| 346 | NGC PAM CP5 variant (S. | monomer_Y147T_Q154S |
| | pyogenes Cas9)_ABE8.12 | |
| 347 | NGC PAM CP5 variant (S. | monomer_H123Y123H_ |
| | pyogenes Cas9)_ABE8.13 | Y147R_Q154R_I76Y |
| 348 | NGC PAM CP5 variant (S. | monomer_deletion at TadA7.10* |
| | pyogenes Cas9)_ABE7.10 | residue 149 |
| 349 | NGC PAM CP5 variant (S. | monomer_deletion at TadA7.10* |
| | pyogenes Cas9)_ABE7.10 | residue 150 |
| 350 | NGC PAM CP5 variant (S. | monomer_deletion at TadA7.10* |
| | pyogenes Cas9)_ABE7.10 | residue 151 |
| 351 | NGC PAM CP5 variant (S. | monomer_deletion at TadA7.10* |
| | pyogenes Cas9)_ABE7.10 | residue 152 |
| 352 | NGC PAM CP5 variant (S. | monomer_deletion at TadA7.10* |
| | pyogenes Cas9)_ABE7.10 | residue 153 |
| 353 | NGC PAM CP5 variant (S. | monomer_deletion at TadA7.10* |
| | pyogenes Cas9)_ABE7.10 | residue 154 |
| 354 | NGC PAM CP5 variant (S. | monomer_deletion at TadA7.10* |
| | pyogenes Cas9)_ABE7.10 | residue 155 |
| 355 | NGC PAM CP5 variant (S. | monomer_deletion at TadA7.10* |
| | pyogenes Cas9)_ABE7.10 | residue 156 |
| 356 | NGC PAM CP5 variant (S. | monomer_deletion at TadA7.10* |
| | pyogenes Cas9)_ABE7.10 | residue 157 |
| 357 | NGC PAM CP5 variant (S. | heterodimer_TadA*7.10 + |
| | pyogenes Cas9)_ABE8.14 | Y147T |
| 358 | NGC PAM CP5 variant (S. | heterodimer_TadA*7.10 + |
| | pyogenes Cas9)_ABE8.15 | Y147R |
| 359 | NGC PAM CP5 variant (S. | heterodimer_TadA*7.10 + Q154S |
| | pyogenes Cas9)_ABE8.16 | |
| 360 | NGC PAM CP5 variant (S. | heterodimer_TadA*7.10 + |
| | pyogenes Cas9)_ABE8.17 | Y123H |
| 361 | NGC PAM C | heterodimer_TadA*7.10 + V82S |
| | P5 variant (S. pyogenes | |
| | Cas9)_ABE8.18 | |
| 362 | NGC PAM CP5 variant (S. | heterodimer_TadA*7.10 + T166R |
| | pyogenes Cas9)_ABE8.19 | |
| 363 | NGC PAM CP5 variant (S. | heterodimer_TadA*7.10 + |
| | pyogenes Cas9)_ABE8.20 | Q154R |
| 364 | NGC PAM CP5 variant (S. | heterodimer_ |
| | pyogenes Cas9)_ABE8.21 | Y147R_Q154R_Y123H |
| 365 | NGC PAM CP5 variant (S. | heterodimer_ |
| | pyogenes Cas9)_ABE8.22 | Y147R_Q154R_I76Y |
| 366 | NGC PAM CP5 variant (S. | heterodimer_ |
| | pyogenes Cas9)_ABE8.23 | Y147R_Q154R_T166R |
| 367 | NGC PAM CP5 variant (S. | heterodimer_Y147T_Q154R |
| | pyogenes Cas9)_ABE8.24 | |
| 368 | NGC PAM CP5 variant (S. | heterodimer_Y147T_Q154S |
| | pyogenes Cas9)_ABE8.25 | |
| 369 | NGC PAM CP5 variant (S. | heterodimer_H123Y123H_ |
| | pyogenes Cas9)_ABE8.26 | Y147R_Q154R_I76Y |
| 370 | NGC PAM CP5 variant (S. | heterodimer_deletion at |
| | pyogenes Cas9)_ABE7.10 | TadA7.10* residue 149 |
| 371 | NGC PAM CP5 variant (S. | heterodimer_deletion at |
| | pyogenes Cas9)_ABE7.10 | TadA7.10* residue 150 |
| 372 | NGC PAM CP5 variant (S. | heterodimer_deletion at |
| | pyogenes Cas9)_ABE7.10 | TadA7.10* residue 151 |
| 373 | NGC PAM CP5 variant (S. | heterodimer_deletion at |
| | pyogenes Cas9)_ABE7.10 | TadA7.10* residue 152 |
| 374 | NGC PAM CP5 variant (S. | heterodimer_deletion at |
| | pyogenes Cas9)_ABE7.10 | TadA7.10* residue 153 |
| 375 | NGC PAM CP5 variant (S. | heterodimer_deletion at |
| | pyogenes Cas9)_ABE7.10 | TadA7.10* residue 154 |
| 376 | NGC PAM CP5 variant (S. | heterodimer_deletion at |
| | pyogenes Cas9)_ABE7.10 | TadA7.10* residue 155 |
| 377 | NGC PAM CP5 variant (S. | heterodimer_deletion at |
| | pyogenes Cas9)_ABE7.10 | TadA7.10* residue 156 |
| 378 | NGC PAM CP5 variant (S. | heterodimer_deletion at |
| | pyogenes Cas9)_ABE7.10 | TadA7.10* residue 157 |

### Example 2: Development of New Adenosine Deaminase Variants

Experiments were undertaken to identify adenosine deaminase variants with improved characteristics and suitable for use in the methods provided herein. The base editors listed in Table 9 were designed and the editing efficiencies thereof were evaluated in vivo.

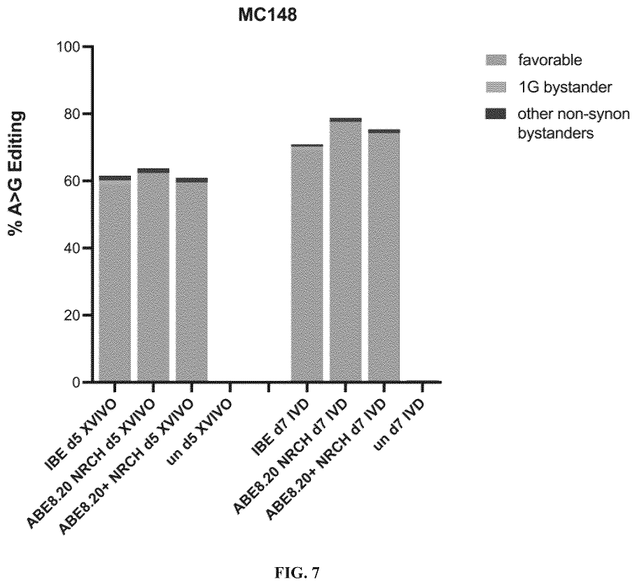

First, experiments were undertaking to evaluate the efficiency of base editing of hematopoietic stem cells containing a Sickle Cell Disease mutation (E6V) in the beta hemoglobin gene (HbSS CD34s). The following base editor systems (see FIG. 7) were used: An Inlaid Base Editor (IBE) in combination with sgRNA_017; ABE8.20-NRCH in combination with sgRNA_027; and ABE8.20+ in combination with sgRNA_027. The term “NRCH” refers to the PAM recognized by the Cas9 variant within the indicated base editor, where N represents A, C, G, or T, R represents A or G, and H represents A, C, or T. The amino acid and/or nucleotide sequences for ABE8.20-NRCH, ABE8.20+, and the Inlaid Base Editor (IBE) referenced in FIG. 7 are provided in Table 10 below. The ABE8.20-NRCH base editor contained a TadA*8.20 deaminase domain, the ABE9v1-NRCH base editor contained a TadA*8.20 deaminase domain with the amino acid alteration S82T, and the ABE9v2 base editor contained a TadA*8.20 deaminase domain with the amino acid alterations S82T, Y147D, T166I, and D167N. As shown in FIG. 7, it was found that Makassar editing (i.e., installing the Makassar edit or the Ser9Pro bystander and other non-synonymous bystanders within the target window on the beta globin gene) was achieved at the beta globin locus in the CD34+ cells grown in XVIVO medium (serum-free stem cell medium) 5 days (i.e., “d5”) post electroporation (EP) and 7 days (i.e., “d7”) in in vitro differentiated erythroid cultures (IVD).

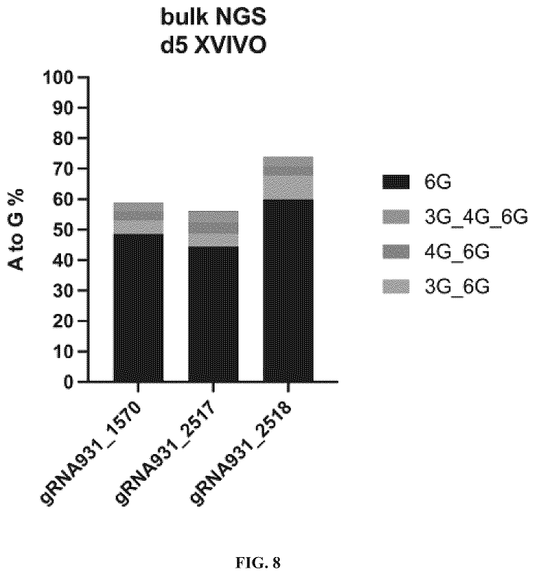

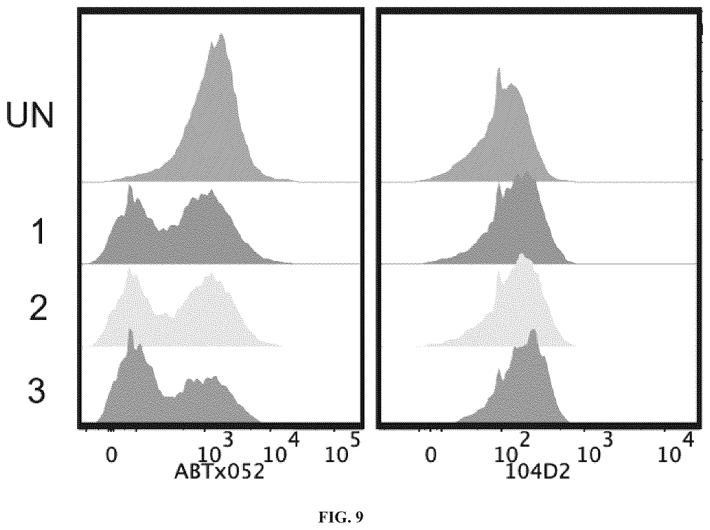

Experiments were undertaken to evaluate editing efficiencies in CD34+ hematopoietic stem cells (HSCs) using the base editors ABE8.20, ABE9v1, and ABE9v2 (see Table 10) in combination with the guide gRNA931, which contained the spacer sequence AUAAUAGCUGGCAUCACGGU (SEQ ID NO: 481) and the scaffold sequence GUUUUAGAGCUAGAAAUAGCAAGUUAAAAUAAGGCUAGUCCGUUAUCAACUUGAAAAAGUGG CACCGAGUCGGUGCUUUU (SEQ ID NO: 317), and which targeted the base editors to effect a nucleobase alteration(s) to a polynucleotide encoding a CD117 polynucleotide. The cells were transfected with the guide and mRNA encoding the base editors using electroporation. The cells were grown in XVIVO serum free stem cell medium. All of the combinations evaluated resulted in editing efficiencies of greater than 50% (see FIG. 8). The alteration to the CD117 polynucleotide resulted in an amino acid alteration to the encoded CD117 polypeptide that resulted in reduced binding to the antibody ABTx052 so that cells expressing the altered CD117 polypeptide showed reduced binding to the antibody (FIG. 9).

### Example 3: Engraftment of Base Edited Human CD34+ Cells in NBSGW Mice

Experiments were undertaken to evaluate the engraftment in immunocompromised 6-8 weeks-old female NBSGW mice (NOD.Cg-Kit^{W-41J}Tyr⁺ Prkdc^{scid} Il2rg^{tm1Wjl}/ThomJ) of human CD34 expressing cells (hCD34+ cells) edited according to the methods of the disclosure.

First, a hemoglobin beta (HBB) gene in the hCD34+ cells was edited by electroporating the cells with a base editor system containing the guide polynucleotide gRNA2861 containing the spacer UUCUCCACAGGAGUCAGGUG (SEQ ID NO: 445) and the mRNA molecule mRNA2518 encoding the base editor ABE9v2 (see amino acid and nucleotide sequences provided in Table 9 and the mRNA2518 nucleotide sequence provided below). The base edited cells contained a “Makassar edit” resulting in expression of a beta globin polypeptide having an alanine at position 6 (Hb G-Makassar). A dose of 1e6 of the edited hCD34+ cells was then transplanted into 15 NBSGW mice at day zero (Group 1 of mice). As a control, 15 NBSGW mice were also transplanted with unedited hCD34+ cells at day zero (Group 2 of mice). At 8 weeks post-transplantation (8 wk Take Down (TD)) of the hCD34+ cells, five mice from each group were euthanized and engraftment of the hCD34+ cells were measured. At 16 weeks post-transplantation (16 wk Take Down (TD)) of the hCD34+ cells, ten mice from each group were euthanized and engraftment of the hCD34+ cells was measured. Bone marrow harvested from the pelvic bones, tibia, and femurs was evaluated. Engraftment of the hCD34+ cells was measured using the following parameters: percent human CD45+(% hCD45); frequencies of cell populations measured using flow cytometry; base edits observed in bulk bone marrow; and base editing observed in sorted (hCD15+, hCD19+, hCD235a+, and lin-CD34+) cell populations.

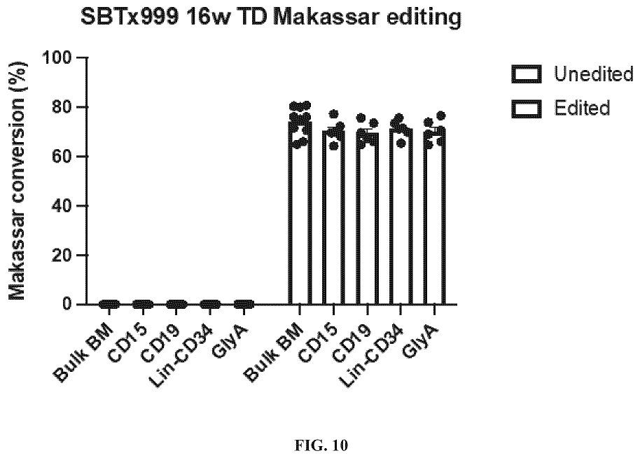

At 16 weeks post-transplantation, it was observed that both bulk bone marrow and bone marrow cell sub-populations in the mice transplanted with the edited hCD34+ cells contained hCD34+ cells containing base edited beta globin polynucleotides containing one of the following base modifications (FIG. 10): 9G, 9G+11G, 9G+11G+14G, 5T+9G+11G, where the numbers indicate the nucleotide location of each alteration, and where each alteration is indicated relative to the following sequence, where subscripts indicate the nucleotide locations: ACTTC₅TCCA₉CA₁₁GGA₁₄GTCAGATGC (SEQ ID NO: 439). The target base edit was 9G. The hCD15+, hCD19+, Lin-hCD34+, and GlyA cells were sorted from the bone marrow samples using fluorescence-activated cell sorting (FACS). Base modifications were detected using next-generation sequencing. This data demonstrated that the nucleobase alterations (“Makassar edits”) in the base edited hCD34+ cells were retained in both bulk bone marrow and sorted cell subpopulations at 16 weeks post-transplant.

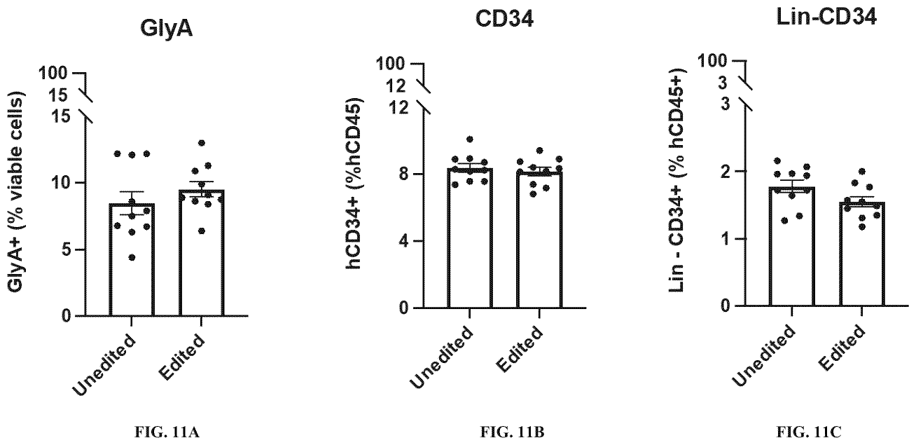

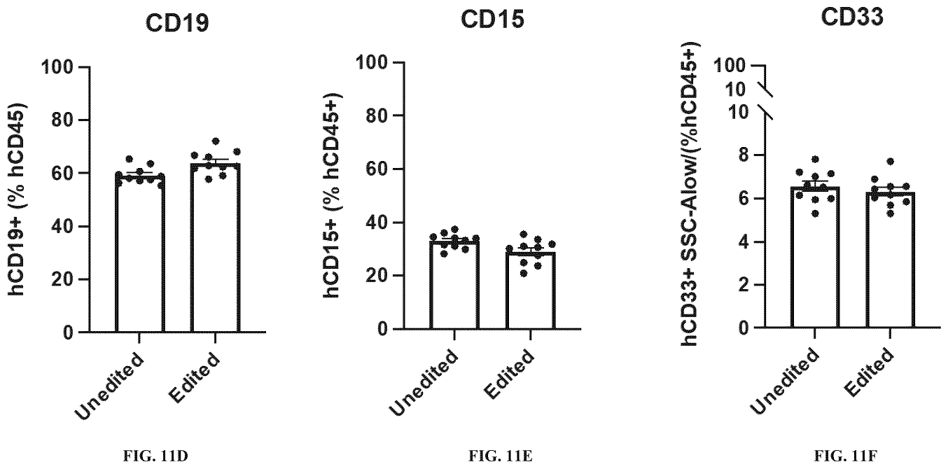

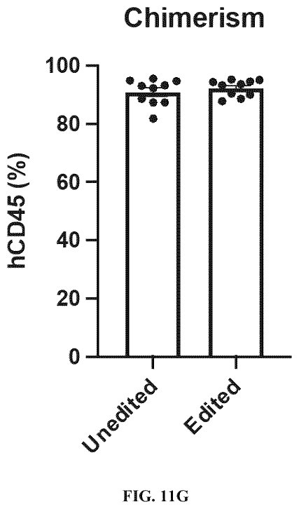

Frequencies of cell populations including GlyA, hCD34, Lin(−) hCD34, hCD19, hCD15, hCD33, and hCD45 and chimerism was comparable between edited and edited hCD34+ cells transplanted into the mice (FIGS. 11A-11G), which demonstrated that base editing did not alter engraftment or multilineage reconstitution potential of the hCD34+ cells.

The following materials and methods were employed in the above examples.

### Sequences

Table 9 provides a list of amino acid and nucleotide sequences corresponding to base editors used in the Examples.

TABLE 9
Base editor sequences. In the amino acid sequences, adenosine deaminase
domains are in plain text, linkers are in italics, nucleic acid programmable DNA
binding protein (e.g., Cas9-NRCH or Cas9- MQKFRAER*) domains are in bold, and
bipartite nuclear localization signals are underlined. The Cas9-NRCH domains
recognized the PAM sequence CACC. In embodiments, the ABE8.20-NRCH and
ABE8.20+-NRCH base editors avoid introducing a Ser9Pro bystander edit to a beta
globin gene sequence.

| Editor | Amino Acid or Nucleotide Sequence |
|---|---|
| ABE8.20 | MSEVEFSHEYWMRHALTLAKRARDEREVPVGAVLVLNNRVIGEGWNRAIGLHDPTAHAEIM |
| (F149Y)- | ALRQGGLVMQNYRLYDATLYSTFEPCVMCAGAMIHSRIGRVVFGVRNAKTGAAGSLMDVLH |
| NRCH | HPGMNHRVEITEGILADECAALLCRFYRMPRRVFNAQKKAQSSTDSGGSSGGSSGSETPGT |
| | SESATPESSGGSSGGSDKKYSIGLAIGTNSVGWAVITDEYKVPSKKFKVLGNTDRHSIKKN |
| | LIGALLFDSGETAEATRLKRTARRRYTRRKNRICYLQEIFSNEMAKVDDSFFHRLEESFLV |
| | EEDKKHERHPIFGNIVDEVAYHEKYPTIYHLRKKLVDSTDKADLRLIYLALAHMIKFRGHF |
| | LIEGDLNPDNSDVDKLFIQLVQTYNQLFEENPINASGVDAKAILSARLSKSRRLENLIAQL |
| | PGEKKNGLFGNLIALSLGLTPNFKSNFDLAEDAKLQLSKDTYDDDLDNLLAQIGDQYADLF |
| | LAAKNLSDAILLSDILRVNTEITKAPLSASMVKRYDEHHQDLTLLKALVRQQLPEKYKEIF |
| | FDQSKNGYAGYIDGGASQEEFYKFIKPILEKMDGTEELLVKLNREDLLRKQRTFDNGIIPH |
| | QIHLGELHAILRRQGDFYPFLKDNREKIEKILTFRIPYYVGPLARGNSRFAWMTRKSEETI |
| | TPWNFEEVVDKGASAQSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEISGVEDRFNASLGTYH |
| | DLLKIIKDKDFLDNEENEDILEDIVLTLTLFEDREMIEERLKTYAHLFDDKVMKQLKRLRY |
| | TGWGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGGHKPENIVIEMARENQTTQKGQKNSR |
| | ERMKRIEEGIKELGSQILKEHPVENTQLQNEKLYLYYLQNGRDMYVDQELDINRLSDYDVD |
| | HIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSK |
| | LVSDFRKDFQFYKVREINNYHHAHDAYLNAVVGTALIKKYPKLESEFVYGDYKVYDVRKMI |
| | AKSEQEIGKATAKYFFYSNIMNFFKTEITLANGEIRKRPLIETNGETGEIVWDKGRDFATV |
| | RKVLSMPQVNIVKKTEVQTGGFSKESILPKGNSDKLIARKKDWDPKKYGGFNSPTVAYSVL |
| | VVAKVEKGKSKKLKSVKELLGITIMERSSFEKNPIDFLEAKGYKEVKKDLIIKLPKYSLFE |
| | LENGRKRMLASAGVLQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYF |
| | DTTINRKQYNTTKEVLDATLIRQSITGLYETRIDLSQLGGDEGADKRTADGSEFESPKKKR |
| | KV (SEQ ID NO: 482) |
| ABE8.20- | TAATACGACTCACTATAAGGAAATAAGAGAGAAAAGAAGAGTAAGAAGAAATATAAGAGCC |
| NRCH | ACCATGTCCGAAGTCGAGTTTTCCCATGAGTACTGGATGAGACACGCATTGACTCTCGCAA |
| | AGAGGGCTCGAGATGAACGCGAGGTGCCCGTGGGGGCAGTACTCGTGCTCAACAATCGCGT |
| | AATCGGCGAAGGTTGGAATAGGGCAATCGGACTCCACGACCCCACTGCACATGCGGAAATC |
| | ATGGCCCTTCGACAGGGAGGGCTTGTGATGCAGAATTATCGACTTTATGATGCGACGCTGT |
| | ACTCGACGTTTGAACCTTGCGTAATGTGCGCGGGAGCTATGATTCACTCCCGCATTGGACG |
| | AGTTGTATTCGGTGTTCGCAACGCCAAGACGGGTGCCGCAGGTTCACTGATGGACGTGCTG |
| | CATCATCCAGGCATGAACCACCGGGTAGAAATCACAGAAGGCATATTGGCGGACGAATGTG |
| | CGGCGCTGTTGTGTCGTTTTTATCGCATGCCCAGGCGTGTCTTTAACGCCCAGAAAAAAGC |
| | ACAATCCTCTACTGACTCCGGCGGAAGCAGCGGAGGATCTTCTGGAAGCGAAACCCCAGGC |
| | ACCAGCGAGTCTGCCACACCAGAATCATCTGGCGGTAGCTCCGGCGGCAGCGACAAGAAGT |
| | ATTCTATCGGACTGGCCATCGGCACCAACTCTGTTGGATGGGCCGTGATCACCGACGAGTA |
| | CAAGGTGCCCAGCAAGAAATTCAAGGTGCTGGGCAACACCGACAGGCACAGCATCAAGAAG |
| | AACCTGATCGGCGCACTGCTGTTCGACTCTGGCGAAACAGCCGAGGCCACCAGACTGAAGA |
| | GAACAGCCCGCAGACGGTACACCAGAAGAAAGAACCGGATCTGCTACCTCCAAGAGATCTT |
| | CAGCAACGAGATGGCCAAGGTGGACGACAGCTTCTTCCACAGACTGGAAGAGTCCTTCCTG |
| | GTGGAAGAGGACAAGAAGCACGAGAGACACCCCATCTTCGGCAACATCGTGGACGAGGTGG |
| | CCTACCACGAGAAGTACCCCACCATCTACCACCTGAGAAAGAAACTGGTGGACAGCACCGA |
| | CAAGGCCGACCTGAGACTGATCTATCTGGCCCTGGCTCACATGATCAAGTTCCGGGGCCAC |
| | TTCCTGATCGAGGGCGACCTGAATCCTGACAACAGCGACGTGGACAAGCTGTTCATCCAGC |
| | TGGTGCAGACCTACAACCAGCTGTTCGAGGAAAACCCCATCAACGCCAGCGGAGTGGATGC |
| | CAAGGCCATCCTGTCTGCCAGACTGAGCAAGAGCAGACGGCTGGAAAATCTGATCGCCCAG |
| | CTGCCTGGCGAGAAGAAGAATGGCCTGTTCGGCAACCTGATTGCCCTGAGCCTGGGCCTGA |
| | CACCTAACTTCAAGAGCAACTTCGACCTGGCCGAGGACGCCAAACTGCAGCTGAGCAAGGA |
| | CACCTACGACGACGACCTGGACAATCTGCTGGCCCAGATCGGCGATCAGTACGCCGACTTG |
| | TTTCTGGCCGCCAAGAATCTGAGCGACGCCATCCTGCTGTCCGACATCCTGAGAGTGAACA |
| | CCGAGATCACCAAGGCACCTCTGAGCGCCTCTATGGTCAAGAGATACGACGAGCACCACCA |
| | GGATCTGACCCTGCTGAAGGCCCTCGTTAGACAGCAGCTGCCAGAGAAGTACAAAGAGATT |
| | TTCTTCGACCAGAGCAAGAACGGCTACGCCGGCTACATTGATGGCGGAGCCAGCCAAGAGG |
| | AATTCTACAAGTTCATCAAGCCCATCCTCGAGAAGATGGACGGCACCGAGGAACTGCTGGT |
| | CAAGCTGAACAGAGAGGACCTGCTGAGAAAGCAGAGAACCTTCGACAACGGCATCATCCCT |
| | CACCAGATCCACCTGGGAGAACTGCACGCCATTCTGCGGAGACAAGGGGACTTTTACCCAT |
| | TCCTGAAGGACAACCGGGAAAAGATCGAGAAAATCCTGACCTTCAGGATCCCCTACTACGT |
| | GGGACCACTGGCCAGAGGCAATAGCAGATTCGCCTGGATGACCAGAAAGAGCGAGGAAACC |
| | ATCACTCCCTGGAACTTCGAGGAAGTGGTGGACAAGGGCGCCAGCGCTCAGTCCTTCATCG |
| | AGCGGATGACCAACTTCGATAAGAACCTGCCTAACGAGAAGGTGCTGCCCAAGCACAGCCT |
| | GCTGTACGAGTACTTCACCGTGTACAACGAGCTGACCAAAGTGAAATACGTGACCGAGGGA |
| | ATGAGAAAGCCCGCCTTTCTGAGCGGCGAGCAGAAAAAGGCCATCGTGGATCTGCTGTTCA |
| | AGACCAACCGGAAAGTGACCGTGAAGCAGCTGAAAGAGGACTACTTCAAGAAAATCGAGTG |
| | CTTCGACAGCGTCGAGATCTCCGGCGTGGAAGATCGGTTCAATGCCAGCCTGGGCACATAC |
| | CACGATCTGCTGAAAATTATCAAGGACAAGGACTTCCTGGACAACGAAGAGAACGAGGACA |
| | TCCTTGAGGACATCGTGCTGACACTGACCCTGTTTGAGGACAGAGAGATGATCGAGGAACG |
| | GCTGAAAACATACGCCCACCTGTTCGACGACAAAGTGATGAAGCAACTGAAGCGGCTGAGA |
| | TACACCGGCTGGGGCAGACTGTCTCGGAAGCTGATCAACGGCATCCGGGATAAGCAGTCCG |
| | GCAAGACCATCCTGGACTTTCTGAAGTCCGACGGCTTCGCCAACAGAAACTTCATGCAGCT |
| | GATTCACGACGACAGCCTCACCTTCAAAGAGGATATCCAGAAAGCCCAGGTGTCCGGCCAG |
| | GGCGATTCTCTGCATGAGCACATTGCCAACCTGGCCGGCTCTCCCGCCATTAAGAAAGGCA |
| | TCCTGCAGACAGTGAAGGTGGTGGACGAGCTTGTGAAAGTGATGGGCGGACACAAGCCCGA |
| | GAACATCGTGATCGAAATGGCCAGAGAGAACCAGACCACACAGAAGGGACAGAAGAACAGC |
| | CGCGAGAGAATGAAGCGGATCGAAGAGGGCATCAAAGAGCTGGGCAGCCAGATCCTGAAAG |
| | AACACCCCGTGGAAAACACCCAGCTGCAGAACGAGAAGCTGTACCTGTACTACCTGCAGAA |
| | TGGACGGGATATGTACGTGGACCAAGAGCTGGACATCAACAGACTGTCCGACTACGATGTG |
| | GACCATATCGTGCCCCAGTCTTTTCTGAAGGACGACTCCATCGACAACAAGGTCCTGACCA |
| | GATCCGACAAGAATCGGGGCAAGAGCGACAACGTGCCCTCCGAAGAGGTGGTCAAGAAGAT |
| | GAAGAACTACTGGCGACAGCTGCTGAACGCCAAGCTGATTACCCAGCGGAAGTTCGACAAT |
| | CTGACCAAGGCCGAAAGAGGCGGCCTGAGCGAACTGGATAAGGCCGGCTTCATCAAGAGAC |
| | AGCTGGTGGAAACCCGGCAGATCACAAAGCACGTGGCACAGATTCTGGACTCTCGGATGAA |
| | CACTAAGTACGACGAGAACGACAAACTGATCCGCGAAGTGAAAGTCATCACCCTGAAGTCC |
| | AAGCTGGTGTCCGATTTCCGGAAGGATTTCCAGTTCTACAAAGTGCGCGAGATCAACAACT |
| | ACCATCACGCCCACGACGCCTACCTGAATGCCGTTGTTGGAACAGCCCTGATCAAAAAGTA |
| | CCCTAAGCTGGAAAGCGAGTTCGTGTACGGCGACTACAAGGTGTACGACGTGCGGAAGATG |
| | ATCGCCAAGAGCGAGCAAGAGATTGGCAAGGCAACCGCCAAGTACTTCTTCTACAGCAACA |
| | TCATGAACTTTTTCAAGACAGAGATCACCCTCGCCAACGGCGAGATCAGAAAGCGGCCTCT |
| | GATCGAGACAAACGGCGAAACCGGCGAGATTGTGTGGGATAAGGGCAGAGACTTTGCCACA |
| | GTGCGGAAAGTGCTGAGCATGCCCCAAGTGAATATCGTGAAGAAAACCGAGGTGCAGACAG |
| | GCGGCTTCAGCAAAGAGTCTATCCTGCCTAAGGGGAACTCCGACAAGCTGATCGCCAGAAA |
| | GAAGGACTGGGACCCCAAGAAATACGGCGGCTTTAACTCTCCCACCGTGGCCTATTCTGTT |
| | CTGGTGGTGGCCAAAGTGGAAAAGGGCAAGTCCAAGAAACTCAAGAGCGTGAAAGAGCTGC |
| | TGGGGATCACCATCATGGAAAGAAGCAGCTTCGAGAAGAATCCGATCGATTTCCTCGAGGC |
| | CAAGGGTTACAAAGAAGTGAAAAAGGACCTGATCATCAAGCTCCCCAAGTACTCCCTGTTC |
| | GAGCTGGAAAACGGCCGGAAGAGAATGCTGGCCTCTGCCGGTGTCCTGCAGAAGGGAAACG |
| | AACTGGCCCTGCCTAGCAAATATGTGAACTTCCTGTACCTGGCCAGCCACTATGAGAAGCT |
| | GAAGGGCAGCCCCGAGGACAATGAGCAAAAGCAGCTGTTTGTGGAACAGCACAAGCACTAC |
| | CTGGACGAGATCATCGAGCAGATCAGCGAGTTTAGCAAGAGAGTGATTCTGGCCGACGCCA |
| | ATCTGGACAAAGTGCTGTCCGCCTACAACAAGCACCGGGACAAGCCTATCAGAGAGCAGGC |
| | CGAGAATATCATCCACCTGTTTACCCTGACCAACCTGGGAGCCCCTGCGGCCTTCAAGTAC |
| | TTTGACACCACCATCAACAGGAAGCAGTACAACACCACCAAAGAGGTGCTGGACGCCACTC |
| | TGATCCGCCAGTCTATCACCGGCCTGTACGAGACACGGATCGACCTGTCTCAACTCGGAGG |
| | CGACGAAGGCGCCGATAAGAGAACCGCCGATGGCTCTGAGTTCGAGAGCCCTAAGAAAAAG |
| | CGCAAAGTGTAG (SEQ ID NO: 483) |
| ABE8.20+ | MSEVEFSHEYWMRHALTLAKRARDEREVPVGAVLVLNNRVIGEGWNRAIGLHDPTAHAEIM |
| (ABE8.20 | ALRQGGLVMQNYRLYDATLYSTFEPCVMCAGAMIHSRIGRVVFGVRNAKTGAAGSLMDVLH |
| w/ the | HPGMNHRVEITEGILADECAALLCDFYRMPRRVFNAQKKAQSSINSGGSSGGSSGSETPGT |
| alterations | SESATPESSGGSSGGSDKKYSIGLAIGTNSVGWAVITDEYKVPSKKFKVLGNTDRHSIKKN |
| R147D, | LIGALLFDSGETAEATRLKRTARRRYTRRKNRICYLQEIFSNEMAKVDDSFFHRLEESFLV |
| F149Y, | EEDKKHERHPIFGNIVDEVAYHEKYPTIYHLRKKLVDSTDKADLRLIYLALAHMIKFRGHF |
| T166I, and | LIEGDLNPDNSDVDKLFIQLVQTYNQLFEENPINASGVDAKAILSARLSKSRRLENLIAQL |
| D167N in | PGEKKNGLFGNLIALSLGLTPNFKSNFDLAEDAKLQLSKDTYDDDLDNLLAQIGDQYADLE |
| the TadA* | LAAKNLSDAILLSDILRVNTEITKAPLSASMVKRYDEHHQDLTLLKALVRQQLPEKYKEIF |
| domain) | FDQSKNGYAGYIDGGASQEEFYKFIKPILEKMDGTEELLVKLNREDLLRKQRTFDNGIIPH |
| | QIHLGELHAILRRQGDFYPFLKDNREKIEKILTFRIPYYVGPLARGNSRFAWMTRKSEETI |
| | TPWNFEEVVDKGASAQSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEISGVEDRFNASLGTYH |
| | DLLKIIKDKDFLDNEENEDILEDIVLTLTLFEDREMIEERLKTYAHLFDDKVMKQLKRLRY |
| | TGWGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGGHKPENIVIEMARENQTTQKGQKNSR |
| | ERMKRIEEGIKELGSQILKEHPVENTQLQNEKLYLYYLQNGRDMYVDQELDINRLSDYDVD |
| | HIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSK |
| | LVSDFRKDFQFYKVREINNYHHAHDAYLNAVVGTALIKKYPKLESEFVYGDYKVYDVRKMI |
| | AKSEQEIGKATAKYFFYSNIMNFFKTEITLANGEIRKRPLIETNGETGEIVWDKGRDFATV |
| | RKVLSMPQVNIVKKTEVQTGGFSKESILPKGNSDKLIARKKDWDPKKYGGFNSPTVAYSVL |
| | VVAKVEKGKSKKLKSVKELLGITIMERSSFEKNPIDFLEAKGYKEVKKDLIIKLPKYSLFE |
| | LENGRKRMLASAGVLQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYF |
| | DTTINRKQYNTTKEVLDATLIRQSITGLYETRIDLSQLGGDEGADKRTADGSEFESPKKKR |
| | KV (SEQ ID NO: 484) |
| ABE8.20+ | TAATACGACTCACTATAAGGAAATAAGAGAGAAAAGAAGAGTAAGAAGAAATATAAGAGCC |
| | ACCATGTCCGAAGTCGAGTTTTCCCATGAGTACTGGATGAGACACGCATTGACTCTCGCAA |
| | AGAGGGCTCGAGATGAACGCGAGGTGCCCGTGGGGGCAGTACTCGTGCTCAACAATCGCGT |
| | AATCGGCGAAGGTTGGAATAGGGCAATCGGACTCCACGACCCCACTGCACATGCGGAAATC |
| | ATGGCCCTTCGACAGGGAGGGCTTGTGATGCAGAATTATCGACTTTATGATGCGACGCTGT |
| | ACTCGACGTTTGAACCTTGCGTAATGTGCGCGGGAGCTATGATTCACTCCCGCATTGGACG |
| | AGTTGTATTCGGTGTTCGCAACGCCAAGACGGGTGCCGCAGGTTCACTGATGGACGTGCTG |
| | CATCATCCAGGCATGAACCACCGGGTAGAAATCACAGAAGGCATATTGGCGGACGAATGTG |
| | CGGCGCTGTTGTGTGACTTTTATCGCATGCCCAGGCGTGTCTTTAACGCCCAGAAAAAAGC |
| | ACAATCCTCTATAAACTCCGGCGGAAGCAGCGGAGGATCTTCTGGAAGCGAAACCCCAGGC |
| | ACCAGCGAGTCTGCCACACCAGAATCATCTGGCGGTAGCTCCGGCGGCAGCGACAAGAAGT |
| | ATTCTATCGGACTGGCCATCGGCACCAACTCTGTTGGATGGGCCGTGATCACCGACGAGTA |
| | CAAGGTGCCCAGCAAGAAATTCAAGGTGCTGGGCAACACCGACAGGCACAGCATCAAGAAG |
| | AACCTGATCGGCGCACTGCTGTTCGACTCTGGCGAAACAGCCGAGGCCACCAGACTGAAGA |
| | GAACAGCCCGCAGACGGTACACCAGAAGAAAGAACCGGATCTGCTACCTCCAAGAGATCTT |
| | CAGCAACGAGATGGCCAAGGTGGACGACAGCTTCTTCCACAGACTGGAAGAGTCCTTCCTG |
| | GTGGAAGAGGACAAGAAGCACGAGAGACACCCCATCTTCGGCAACATCGTGGACGAGGTGG |
| | CCTACCACGAGAAGTACCCCACCATCTACCACCTGAGAAAGAAACTGGTGGACAGCACCGA |
| | CAAGGCCGACCTGAGACTGATCTATCTGGCCCTGGCTCACATGATCAAGTTCCGGGGCCAC |
| | TTCCTGATCGAGGGCGACCTGAATCCTGACAACAGCGACGTGGACAAGCTGTTCATCCAGC |
| | TGGTGCAGACCTACAACCAGCTGTTCGAGGAAAACCCCATCAACGCCAGCGGAGTGGATGC |
| | CAAGGCCATCCTGTCTGCCAGACTGAGCAAGAGCAGACGGCTGGAAAATCTGATCGCCCAG |
| | CTGCCTGGCGAGAAGAAGAATGGCCTGTTCGGCAACCTGATTGCCCTGAGCCTGGGCCTGA |
| | CACCTAACTTCAAGAGCAACTTCGACCTGGCCGAGGACGCCAAACTGCAGCTGAGCAAGGA |
| | CACCTACGACGACGACCTGGACAATCTGCTGGCCCAGATCGGCGATCAGTACGCCGACTTG |
| | TTTCTGGCCGCCAAGAATCTGAGCGACGCCATCCTGCTGTCCGACATCCTGAGAGTGAACA |
| | CCGAGATCACCAAGGCACCTCTGAGCGCCTCTATGGTCAAGAGATACGACGAGCACCACCA |
| | GGATCTGACCCTGCTGAAGGCCCTCGTTAGACAGCAGCTGCCAGAGAAGTACAAAGAGATT |
| | TTCTTCGACCAGAGCAAGAACGGCTACGCCGGCTACATTGATGGCGGAGCCAGCCAAGAGG |
| | AATTCTACAAGTTCATCAAGCCCATCCTCGAGAAGATGGACGGCACCGAGGAACTGCTGGT |
| | CAAGCTGAACAGAGAGGACCTGCTGAGAAAGCAGAGAACCTTCGACAACGGCATCATCCCT |
| | CACCAGATCCACCTGGGAGAACTGCACGCCATTCTGCGGAGACAAGGGGACTTTTACCCAT |
| | TCCTGAAGGACAACCGGGAAAAGATCGAGAAAATCCTGACCTTCAGGATCCCCTACTACGT |
| | GGGACCACTGGCCAGAGGCAATAGCAGATTCGCCTGGATGACCAGAAAGAGCGAGGAAACC |
| | ATCACTCCCTGGAACTTCGAGGAAGTGGTGGACAAGGGCGCCAGCGCTCAGTCCTTCATCG |
| | AGCGGATGACCAACTTCGATAAGAACCTGCCTAACGAGAAGGTGCTGCCCAAGCACAGCCT |
| | GCTGTACGAGTACTTCACCGTGTACAACGAGCTGACCAAAGTGAAATACGTGACCGAGGGA |
| | ATGAGAAAGCCCGCCTTTCTGAGCGGCGAGCAGAAAAAGGCCATCGTGGATCTGCTGTTCA |
| | AGACCAACCGGAAAGTGACCGTGAAGCAGCTGAAAGAGGACTACTTCAAGAAAATCGAGTG |
| | CTTCGACAGCGTCGAGATCTCCGGCGTGGAAGATCGGTTCAATGCCAGCCTGGGCACATAC |
| | CACGATCTGCTGAAAATTATCAAGGACAAGGACTTCCTGGACAACGAAGAGAACGAGGACA |
| | TCCTTGAGGACATCGTGCTGACACTGACCCTGTTTGAGGACAGAGAGATGATCGAGGAACG |
| | GCTGAAAACATACGCCCACCTGTTCGACGACAAAGTGATGAAGCAACTGAAGCGGCTGAGA |
| | TACACCGGCTGGGGCAGACTGTCTCGGAAGCTGATCAACGGCATCCGGGATAAGCAGTCCG |
| | GCAAGACCATCCTGGACTTTCTGAAGTCCGACGGCTTCGCCAACAGAAACTTCATGCAGCT |
| | GATTCACGACGACAGCCTCACCTTCAAAGAGGATATCCAGAAAGCCCAGGTGTCCGGCCAG |
| | GGCGATTCTCTGCATGAGCACATTGCCAACCTGGCCGGCTCTCCCGCCATTAAGAAAGGCA |
| | TCCTGCAGACAGTGAAGGTGGTGGACGAGCTTGTGAAAGTGATGGGCGGACACAAGCCCGA |
| | GAACATCGTGATCGAAATGGCCAGAGAGAACCAGACCACACAGAAGGGACAGAAGAACAGC |
| | CGCGAGAGAATGAAGCGGATCGAAGAGGGCATCAAAGAGCTGGGCAGCCAGATCCTGAAAG |
| | AACACCCCGTGGAAAACACCCAGCTGCAGAACGAGAAGCTGTACCTGTACTACCTGCAGAA |
| | TGGACGGGATATGTACGTGGACCAAGAGCTGGACATCAACAGACTGTCCGACTACGATGTG |
| | GACCATATCGTGCCCCAGTCTTTTCTGAAGGACGACTCCATCGACAACAAGGTCCTGACCA |
| | GATCCGACAAGAATCGGGGCAAGAGCGACAACGTGCCCTCCGAAGAGGTGGTCAAGAAGAT |
| | GAAGAACTACTGGCGACAGCTGCTGAACGCCAAGCTGATTACCCAGCGGAAGTTCGACAAT |
| | CTGACCAAGGCCGAAAGAGGCGGCCTGAGCGAACTGGATAAGGCCGGCTTCATCAAGAGAC |
| | AGCTGGTGGAAACCCGGCAGATCACAAAGCACGTGGCACAGATTCTGGACTCTCGGATGAA |
| | CACTAAGTACGACGAGAACGACAAACTGATCCGCGAAGTGAAAGTCATCACCCTGAAGTCC |
| | AAGCTGGTGTCCGATTTCCGGAAGGATTTCCAGTTCTACAAAGTGCGCGAGATCAACAACT |
| | ACCATCACGCCCACGACGCCTACCTGAATGCCGTTGTTGGAACAGCCCTGATCAAAAAGTA |
| | CCCTAAGCTGGAAAGCGAGTTCGTGTACGGCGACTACAAGGTGTACGACGTGCGGAAGATG |
| | ATCGCCAAGAGCGAGCAAGAGATTGGCAAGGCAACCGCCAAGTACTTCTTCTACAGCAACA |
| | TCATGAACTTTTTCAAGACAGAGATCACCCTCGCCAACGGCGAGATCAGAAAGCGGCCTCT |
| | GATCGAGACAAACGGCGAAACCGGCGAGATTGTGTGGGATAAGGGCAGAGACTTTGCCACA |
| | GTGCGGAAAGTGCTGAGCATGCCCCAAGTGAATATCGTGAAGAAAACCGAGGTGCAGACAG |
| | GCGGCTTCAGCAAAGAGTCTATCCTGCCTAAGGGGAACTCCGACAAGCTGATCGCCAGAAA |
| | GAAGGACTGGGACCCCAAGAAATACGGCGGCTTTAACTCTCCCACCGTGGCCTATTCTGTT |
| | CTGGTGGTGGCCAAAGTGGAAAAGGGCAAGTCCAAGAAACTCAAGAGCGTGAAAGAGCTGC |
| | TGGGGATCACCATCATGGAAAGAAGCAGCTTCGAGAAGAATCCGATCGATTTCCTCGAGGC |
| | CAAGGGTTACAAAGAAGTGAAAAAGGACCTGATCATCAAGCTCCCCAAGTACTCCCTGTTC |
| | GAGCTGGAAAACGGCCGGAAGAGAATGCTGGCCTCTGCCGGTGTCCTGCAGAAGGGAAACG |
| | AACTGGCCCTGCCTAGCAAATATGTGAACTTCCTGTACCTGGCCAGCCACTATGAGAAGCT |
| | GAAGGGCAGCCCCGAGGACAATGAGCAAAAGCAGCTGTTTGTGGAACAGCACAAGCACTAC |
| | CTGGACGAGATCATCGAGCAGATCAGCGAGTTTAGCAAGAGAGTGATTCTGGCCGACGCCA |
| | ATCTGGACAAAGTGCTGTCCGCCTACAACAAGCACCGGGACAAGCCTATCAGAGAGCAGGC |
| | CGAGAATATCATCCACCTGTTTACCCTGACCAACCTGGGAGCCCCTGCGGCCTTCAAGTAC |
| | TTTGACACCACCATCAACAGGAAGCAGTACAACACCACCAAAGAGGTGCTGGACGCCACTC |
| | TGATCCGCCAGTCTATCACCGGCCTGTACGAGACACGGATCGACCTGTCTCAACTCGGAGG |
| | CGACGAAGGCGCCGATAAGAGAACCGCCGATGGCTCTGAGTTCGAGAGCCCTAAGAAAAAG |
| | CGCAAAGTGTAG (SEQ ID NO: 485) |
| Inlaid Base | MDKKYSIGLAIGTNSVGWAVITDEYKVPSKKFKVLGNTDRHSIKKNLIGALLFDSGETAEA |
| Editor (IBE) | TRLKRTARRRYTRRKNRICYLQEIFSNEMAKVDDSFFHRLEESFLVEEDKKHERHPIFGNI |
| referenced | VDEVAYHEKYPTIYHLRKKLVDSTDKADLRLIYLALAHMIKFRGHFLIEGDLNPDNSDVDK |
| in FIG. 7 | LFIQLVQTYNQLFEENPINASGVDAKAILSARLSKSRRLENLIAQLPGEKKNGLFGNLIAL |
| (MSP684 | SLGLTPNFKSNFDLAEDAKLQLSKDTYDDDLDNLLAQIGDQYADLFLAAKNLSDAILLSDI |
| monoTadA- | ASQEEFYKFIKPILEKMDGTEELLVKLNREDLLRKQRTFDNGSIPHQIHLGELHAILRRQE |
| TadA7.10 | LRVNTEITKAPLSASMIKRYDEHHQDLTLLKALVRQQLPEKYKEIFFDQSKNGYAGYIDGG |
| (V82S)- | DFYPFLKDNREKIEKILTFRIPYYVGPLARGNSRFAWMTRKSEETITPWNFEEVVDKGASA |
| MQKFRAER*) | QSFIERMTNFDKNLPNEKVLPKHSLLYEYFTVYNELTKVKYVTEGMRKPAFLSGEQKKAIV |
| | DLLFKTNRKVTVKQLKEDYFKKIECFDSVEISGVEDRFNASLGTYHDLLKIIKDKDFLDNE |
| | ENEDILEDIVLTLTLFEDREMIEERLKTYAHLFDDKVMKQLKRRRYTGWGRLSRKLINGIR |
| | DKQSGKTILDFLKSDGFANRNFMQLIHDDSLTFKEDIQKAQVSGQGDSLHEHIANLAGSPA |
| | IKKGILQTVKVVDELVKVMGRHKPENIVIEMARENQTTQKGQKNSRERMKRIEEGIKELGS |
| | QILKEHPVENTQLQNEKLYLYYLQNGRDMYVDQELDINRLSDYDVDHIVPQSFLKDDSIDN |
| | KVLTRSDKNRGKSDNVPSEEVVKKMKNYWRQLLNAKLITQRKFDNLTKAERGGLSELDKAG |
| | FIKRQLVETRQITKHVAQILDSRMNTKYDENDKLIREVKVITLKSKLVSDFRKDFQFYKVR |
| | EINNYHHAHDAYLNAVVGTALIKKYPKLESEFVYGDYKVYDVRKMIAKSEQEIGSSGSETP |
| | GTSESATPESSGSEVEFSHEYWMRHALTLAKRARDEREVPVGAVLVLNNRVIGEGWNRAIG |
| | LHDPTAHAEIMALRQGGLVMQNYRLIDATLYSTFEPCVMCAGAMIHSRIGRVVFGVRNAKT |
| | GAAGSLMDVLHYPGMNHRVEITEGILADECAALLCYFFRMPRQVFNAQKKAQSSTDGKATA |
| | KYFFYSNIMNFFKTEITLANGEIRKRPLIETNGETGEIVWDKGRDFATVRKVLSMPQVNIV |
| | KKTEVQTGGFSKESILPKRNSDKLIARKKDWDPKKYGGFMQPTVAYSVLVVAKVEKGKSKK |
| | LKSVKELLGITIMERSSFEKNPIDFLEAKGYKEVKKDLIIKLPKYSLFELENGRKRMLASA |
| | KFLQKGNELALPSKYVNFLYLASHYEKLKGSPEDNEQKQLFVEQHKHYLDEIIEQISEFSK |
| | RVILADANLDKVLSAYNKHRDKPIREQAENIIHLFTLTNLGAPRAFKYFDTTIARKEYRST |
| | KEVLDATLIHQSITGLYETRIDLSQLGGD (SEQ ID NO: 486) |
| ABE9v1- | MSEVEFSHEYWMRHALTLAKRARDEREVPVGAVLVLNNRVIGEGWNRAIGLHDPTAHAEIM |
| NRCH | ALRQGGLVMQNYRLYDATLYTTFEPCVMCAGAMIHSRIGRVVFGVRNAKTGAAGSLMDVLH |
| (ABE8.20 | HPGMNHRVEITEGILADECAALLCRFYRMPRRVFNAQKKAQSSTDSGGSSGGSSGSETPGT |
| w/ the | SESATPESSGGSSGGSDKKYSIGLAIGTNSVGWAVITDEYKVPSKKFKVLGNTDRHSIKKN |
| alterations | LIGALLFDSGETAEATRLKRTARRRYTRRKNRICYLQEIFSNEMAKVDDSFFHRLEESFLV |
| S82T and | EEDKKHERHPIFGNIVDEVAYHEKYPTIYHLRKKLVDSTDKADLRLIYLALAHMIKFRGHF |
| F149Y in | LIEGDLNPDNSDVDKLFIQLVQTYNQLFEENPINASGVDAKAILSARLSKSRRLENLIAQL |
| the TadA* | PGEKKNGLFGNLIALSLGLTPNFKSNFDLAEDAKLQLSKDTYDDDLDNLLAQIGDQYADLF |
| domain) | LAAKNLSDAILLSDILRVNTEITKAPLSASMVKRYDEHHQDLTLLKALVRQQLPEKYKEIF |
| | FDQSKNGYAGYIDGGASQEEFYKFIKPILEKMDGTEELLVKLNREDLLRKQRTFDNGIIPH |
| | QIHLGELHAILRRQGDFYPFLKDNREKIEKILTFRIPYYVGPLARGNSRFAWMTRKSEETI |
| | TPWNFEEVVDKGASAQSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEISGVEDRFNASLGTYH |
| | DLLKIIKDKDFLDNEENEDILEDIVLTLTLFEDREMIEERLKTYAHLFDDKVMKQLKRLRY |
| | TGWGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGGHKPENIVIEMARENQTTQKGQKNSR |
| | ERMKRIEEGIKELGSQILKEHPVENTQLQNEKLYLYYLQNGRDMYVDQELDINRLSDYDVD |
| | HIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSK |
| | LVSDFRKDFQFYKVREINNYHHAHDAYLNAVVGTALIKKYPKLESEFVYGDYKVYDVRKMI |
| | AKSEQEIGKATAKYFFYSNIMNFFKTEITLANGEIRKRPLIETNGETGEIVWDKGRDFATV |
| | RKVLSMPQVNIVKKTEVQTGGFSKESILPKGNSDKLIARKKDWDPKKYGGFNSPTVAYSVL |
| | VVAKVEKGKSKKLKSVKELLGITIMERSSFEKNPIDFLEAKGYKEVKKDLIIKLPKYSLFE |
| | LENGRKRMLASAGVLQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYF |
| | DTTINRKQYNTTKEVLDATLIRQSITGLYETRIDLSQLGGDEGADKRTADGSEFESPKKKR |
| | KV (SEQ ID NO: 487) |
| ABE9v1- | ATGTCCGAAGTCGAGTTTTCCCATGAGTACTGGATGAGACACGCATTGACTCTCGCAAAGA |
| NRCH | GGGCTCGAGATGAACGCGAGGTGCCCGTGGGGGCAGTACTCGTGCTCAACAATCGCGTAAT |
| | CGGCGAAGGTTGGAATAGGGCAATCGGACTCCACGACCCCACTGCACATGCGGAAATCATG |
| | GCCCTTCGACAGGGAGGGCTTGTGATGCAGAATTATCGACTTTATGATGCGACGCTGTACA |
| | CCACGTTTGAACCTTGCGTAATGTGCGCGGGAGCTATGATTCACTCCCGCATTGGACGAGT |
| | TGTATTCGGTGTTCGCAACGCCAAGACGGGTGCCGCAGGTTCACTGATGGACGTGCTGCAT |
| | CATCCAGGCATGAACCACCGGGTAGAAATCACAGAAGGCATATTGGCGGACGAATGTGCGG |
| | CGCTGTTGTGTCGTTTTTATCGCATGCCCAGGCGTGTCTTTAACGCCCAGAAAAAAGCACA |
| | ATCCTCTACTGACTCCGGCGGAAGCAGCGGAGGATCTTCTGGAAGCGAAACCCCAGGCACC |
| | AGCGAGTCTGCCACACCAGAATCATCTGGCGGTAGCTCCGGCGGCAGCGACAAGAAGTATT |
| | CTATCGGACTGGCCATCGGCACCAACTCTGTTGGATGGGCCGTGATCACCGACGAGTACAA |
| | GGTGCCCAGCAAGAAATTCAAGGTGCTGGGCAACACCGACAGGCACAGCATCAAGAAGAAC |
| | CTGATCGGCGCACTGCTGTTCGACTCTGGCGAAACAGCCGAGGCCACCAGACTGAAGAGAA |
| | CAGCCCGCAGACGGTACACCAGAAGAAAGAACCGGATCTGCTACCTCCAAGAGATCTTCAG |
| | CAACGAGATGGCCAAGGTGGACGACAGCTTCTTCCACAGACTGGAAGAGTCCTTCCTGGTG |
| | GAAGAGGACAAGAAGCACGAGAGACACCCCATCTTCGGCAACATCGTGGACGAGGTGGCCT |
| | ACCACGAGAAGTACCCCACCATCTACCACCTGAGAAAGAAACTGGTGGACAGCACCGACAA |
| | GGCCGACCTGAGACTGATCTATCTGGCCCTGGCTCACATGATCAAGTTCCGGGGCCACTTC |
| | CTGATCGAGGGCGACCTGAATCCTGACAACAGCGACGTGGACAAGCTGTTCATCCAGCTGG |
| | TGCAGACCTACAACCAGCTGTTCGAGGAAAACCCCATCAACGCCAGCGGAGTGGATGCCAA |
| | GGCCATCCTGTCTGCCAGACTGAGCAAGAGCAGACGGCTGGAAAATCTGATCGCCCAGCTG |
| | CCTGGCGAGAAGAAGAATGGCCTGTTCGGCAACCTGATTGCCCTGAGCCTGGGCCTGACAC |
| | CTAACTTCAAGAGCAACTTCGACCTGGCCGAGGACGCCAAACTGCAGCTGAGCAAGGACAC |
| | CTACGACGACGACCTGGACAATCTGCTGGCCCAGATCGGCGATCAGTACGCCGACTTGTTT |
| | CTGGCCGCCAAGAATCTGAGCGACGCCATCCTGCTGTCCGACATCCTGAGAGTGAACACCG |
| | AGATCACCAAGGCACCTCTGAGCGCCTCTATGGTCAAGAGATACGACGAGCACCACCAGGA |
| | TCTGACCCTGCTGAAGGCCCTCGTTAGACAGCAGCTGCCAGAGAAGTACAAAGAGATTTTC |
| | TTCGACCAGAGCAAGAACGGCTACGCCGGCTACATTGATGGCGGAGCCAGCCAAGAGGAAT |
| | TCTACAAGTTCATCAAGCCCATCCTCGAGAAGATGGACGGCACCGAGGAACTGCTGGTCAA |
| | GCTGAACAGAGAGGACCTGCTGAGAAAGCAGAGAACCTTCGACAACGGCATCATCCCTCAC |
| | CAGATCCACCTGGGAGAACTGCACGCCATTCTGCGGAGACAAGGGGACTTTTACCCATTCC |
| | TGAAGGACAACCGGGAAAAGATCGAGAAAATCCTGACCTTCAGGATCCCCTACTACGTGGG |
| | ACCACTGGCCAGAGGCAATAGCAGATTCGCCTGGATGACCAGAAAGAGCGAGGAAACCATC |
| | ACTCCCTGGAACTTCGAGGAAGTGGTGGACAAGGGCGCCAGCGCTCAGTCCTTCATCGAGC |
| | GGATGACCAACTTCGATAAGAACCTGCCTAACGAGAAGGTGCTGCCCAAGCACAGCCTGCT |
| | GTACGAGTACTTCACCGTGTACAACGAGCTGACCAAAGTGAAATACGTGACCGAGGGAATG |
| | AGAAAGCCCGCCTTTCTGAGCGGCGAGCAGAAAAAGGCCATCGTGGATCTGCTGTTCAAGA |
| | CCAACCGGAAAGTGACCGTGAAGCAGCTGAAAGAGGACTACTTCAAGAAAATCGAGTGCTT |
| | CGACAGCGTCGAGATCTCCGGCGTGGAAGATCGGTTCAATGCCAGCCTGGGCACATACCAC |
| | GATCTGCTGAAAATTATCAAGGACAAGGACTTCCTGGACAACGAAGAGAACGAGGACATCC |
| | TTGAGGACATCGTGCTGACACTGACCCTGTTTGAGGACAGAGAGATGATCGAGGAACGGCT |
| | GAAAACATACGCCCACCTGTTCGACGACAAAGTGATGAAGCAACTGAAGCGGCTGAGATAC |
| | ACCGGCTGGGGCAGACTGTCTCGGAAGCTGATCAACGGCATCCGGGATAAGCAGTCCGGCA |
| | AGACCATCCTGGACTTTCTGAAGTCCGACGGCTTCGCCAACAGAAACTTCATGCAGCTGAT |
| | TCACGACGACAGCCTCACCTTCAAAGAGGATATCCAGAAAGCCCAGGTGTCCGGCCAGGGC |
| | GATTCTCTGCATGAGCACATTGCCAACCTGGCCGGCTCTCCCGCCATTAAGAAAGGCATCC |
| | TGCAGACAGTGAAGGTGGTGGACGAGCTTGTGAAAGTGATGGGCGGACACAAGCCCGAGAA |
| | CATCGTGATCGAAATGGCCAGAGAGAACCAGACCACACAGAAGGGACAGAAGAACAGCCGC |
| | GAGAGAATGAAGCGGATCGAAGAGGGCATCAAAGAGCTGGGCAGCCAGATCCTGAAAGAAC |
| | ACCCCGTGGAAAACACCCAGCTGCAGAACGAGAAGCTGTACCTGTACTACCTGCAGAATGG |
| | ACGGGATATGTACGTGGACCAAGAGCTGGACATCAACAGACTGTCCGACTACGATGTGGAC |
| | CATATCGTGCCCCAGTCTTTTCTGAAGGACGACTCCATCGACAACAAGGTCCTGACCAGAT |
| | CCGACAAGAATCGGGGCAAGAGCGACAACGTGCCCTCCGAAGAGGTGGTCAAGAAGATGAA |
| | GAACTACTGGCGACAGCTGCTGAACGCCAAGCTGATTACCCAGCGGAAGTTCGACAATCTG |
| | ACCAAGGCCGAAAGAGGCGGCCTGAGCGAACTGGATAAGGCCGGCTTCATCAAGAGACAGC |
| | TGGTGGAAACCCGGCAGATCACAAAGCACGTGGCACAGATTCTGGACTCTCGGATGAACAC |
| | TAAGTACGACGAGAACGACAAACTGATCCGCGAAGTGAAAGTCATCACCCTGAAGTCCAAG |
| | CTGGTGTCCGATTTCCGGAAGGATTTCCAGTTCTACAAAGTGCGCGAGATCAACAACTACC |
| | ATCACGCCCACGACGCCTACCTGAATGCCGTTGTTGGAACAGCCCTGATCAAAAAGTACCC |
| | TAAGCTGGAAAGCGAGTTCGTGTACGGCGACTACAAGGTGTACGACGTGCGGAAGATGATC |
| | GCCAAGAGCGAGCAAGAGATTGGCAAGGCAACCGCCAAGTACTTCTTCTACAGCAACATCA |
| | TGAACTTTTTCAAGACAGAGATCACCCTCGCCAACGGCGAGATCAGAAAGCGGCCTCTGAT |
| | CGAGACAAACGGCGAAACCGGCGAGATTGTGTGGGATAAGGGCAGAGACTTTGCCACAGTG |
| | CGGAAAGTGCTGAGCATGCCCCAAGTGAATATCGTGAAGAAAACCGAGGTGCAGACAGGCG |
| | GCTTCAGCAAAGAGTCTATCCTGCCTAAGGGGAACTCCGACAAGCTGATCGCCAGAAAGAA |
| | GGACTGGGACCCCAAGAAATACGGCGGCTTTAACTCTCCCACCGTGGCCTATTCTGTTCTG |
| | GTGGTGGCCAAAGTGGAAAAGGGCAAGTCCAAGAAACTCAAGAGCGTGAAAGAGCTGCTGG |
| | GGATCACCATCATGGAAAGAAGCAGCTTCGAGAAGAATCCGATCGATTTCCTCGAGGCCAA |
| | GGGTTACAAAGAAGTGAAAAAGGACCTGATCATCAAGCTCCCCAAGTACTCCCTGTTCGAG |
| | CTGGAAAACGGCCGGAAGAGAATGCTGGCCTCTGCCGGTGTCCTGCAGAAGGGAAACGAAC |
| | TGGCCCTGCCTAGCAAATATGTGAACTTCCTGTACCTGGCCAGCCACTATGAGAAGCTGAA |
| | GGGCAGCCCCGAGGACAATGAGCAAAAGCAGCTGTTTGTGGAACAGCACAAGCACTACCTG |
| | GACGAGATCATCGAGCAGATCAGCGAGTTTAGCAAGAGAGTGATTCTGGCCGACGCCAATC |
| | TGGACAAAGTGCTGTCCGCCTACAACAAGCACCGGGACAAGCCTATCAGAGAGCAGGCCGA |
| | GAATATCATCCACCTGTTTACCCTGACCAACCTGGGAGCCCCTGCGGCCTTCAAGTACTTT |
| | GACACCACCATCAACAGGAAGCAGTACAACACCACCAAAGAGGTGCTGGACGCCACTCTGA |
| | TCCGCCAGTCTATCACCGGCCTGTACGAGACACGGATCGACCTGTCTCAACTCGGAGGCGA |
| | CGAAGGCGCCGATAAGAGAACCGCCGATGGCTCTGAGTTCGAGAGCCCTAAGAAAAAGCGC |
| | AAAGTGTAG (SEQ ID NO: 488) |
| ABE9v2- | MSEVEFSHEYWMRHALTLAKRARDEREVPVGAVLVLNNRVIGEGWNRAIGLHDPTAHAEIM |
| NRCH | ALRQGGLVMQNYRLYDATLYTTFEPCVMCAGAMIHSRIGRVVFGVRNAKTGAAGSLMDVLH |
| (ABE8.20 | HPGMNHRVEITEGILADECAALLCDFYRMPRRVFNAQKKAQSSINSGGSSGGSSGSETPGT |
| w/ the | SESATPESSGGSSGGSDKKYSIGLAIGTNSVGWAVITDEYKVPSKKFKVLGNTDRHSIKKN |
| alterations | LIGALLFDSGETAEATRLKRTARRRYTRRKNRICYLQEIFSNEMAKVDDSFFHRLEESFLV |
| Y147D, | EEDKKHERHPIFGNIVDEVAYHEKYPTIYHLRKKLVDSTDKADLRLIYLALAHMIKFRGHF |
| F149Y, | LIEGDLNPDNSDVDKLFIQLVQTYNQLFEENPINASGVDAKAILSARLSKSRRLENLIAQL |
| T166I, | PGEKKNGLFGNLIALSLGLTPNFKSNFDLAEDAKLQLSKDTYDDDLDNLLAQIGDQYADLF |
| D167N and | LAAKNLSDAILLSDILRVNTEITKAPLSASMVKRYDEHHQDLTLLKALVRQQLPEKYKEIF |
| S82T in the | FDQSKNGYAGYIDGGASQEEFYKFIKPILEKMDGTEELLVKLNREDLLRKQRTFDNGIIPH |
| TadA* | QIHLGELHAILRRQGDFYPFLKDNREKIEKILTFRIPYYVGPLARGNSRFAWMTRKSEETI |
| domain) | TPWNFEEVVDKGASAQSFIERMTNFDKNLPNEKVLPKHSLLYEYFTVYNELTKVKYVTEGM |
| | RKPAFLSGEQKKAIVDLLFKTNRKVTVKQLKEDYFKKIECFDSVEISGVEDRFNASLGTYH |
| | DLLKIIKDKDFLDNEENEDILEDIVLTLTLFEDREMIEERLKTYAHLFDDKVMKQLKRLRY |
| | TGWGRLSRKLINGIRDKQSGKTILDFLKSDGFANRNFMQLIHDDSLTFKEDIQKAQVSGQG |
| | DSLHEHIANLAGSPAIKKGILQTVKVVDELVKVMGGHKPENIVIEMARENQTTQKGQKNSR |
| | ERMKRIEEGIKELGSQILKEHPVENTQLQNEKLYLYYLQNGRDMYVDQELDINRLSDYDVD |
| | HIVPQSFLKDDSIDNKVLTRSDKNRGKSDNVPSEEVVKKMKNYWRQLLNAKLITQRKFDNL |
| | TKAERGGLSELDKAGFIKRQLVETRQITKHVAQILDSRMNTKYDENDKLIREVKVITLKSK |
| | LVSDFRKDFQFYKVREINNYHHAHDAYLNAVVGTALIKKYPKLESEFVYGDYKVYDVRKMI |
| | AKSEQEIGKATAKYFFYSNIMNFFKTEITLANGEIRKRPLIETNGETGEIVWDKGRDFATV |
| | RKVLSMPQVNIVKKTEVQTGGFSKESILPKGNSDKLIARKKDWDPKKYGGFNSPTVAYSVL |
| | VVAKVEKGKSKKLKSVKELLGITIMERSSFEKNPIDFLEAKGYKEVKKDLIIKLPKYSLFE |
| | LENGRKRMLASAGVLQKGNELALPSKYVNFLYLASHYEKLKGSPEDNEQKQLFVEQHKHYL |
| | DEIIEQISEFSKRVILADANLDKVLSAYNKHRDKPIREQAENIIHLFTLTNLGAPAAFKYF |
| | DTTINRKQYNTTKEVLDATLIRQSITGLYETRIDLSQLGGDEGADKRTADGSEFESPKKKR |
| | KV (SEQ ID NO: 489) |
| ABE9v2- | ATGTCCGAAGTCGAGTTTTCCCATGAGTACTGGATGAGACACGCATTGACTCTCGCAAAGA |
| NRCH | GGGCTCGAGATGAACGCGAGGTGCCCGTGGGGGCAGTACTCGTGCTCAACAATCGCGTAAT |
| | CGGCGAAGGTTGGAATAGGGCAATCGGACTCCACGACCCCACTGCACATGCGGAAATCATG |
| | GCCCTTCGACAGGGAGGGCTTGTGATGCAGAATTATCGACTTTATGATGCGACGCTGTACA |
| | CCACGTTTGAACCTTGCGTAATGTGCGCGGGAGCTATGATTCACTCCCGCATTGGACGAGT |
| | TGTATTCGGTGTTCGCAACGCCAAGACGGGTGCCGCAGGTTCACTGATGGACGTGCTGCAT |
| | CATCCAGGCATGAACCACCGGGTAGAAATCACAGAAGGCATATTGGCGGACGAATGTGCGG |
| | CGCTGTTGTGTGACTTTTATCGCATGCCCAGGCGTGTCTTTAACGCCCAGAAAAAAGCACA |
| | ATCCTCTATAAACTCCGGCGGAAGCAGCGGAGGATCTTCTGGAAGCGAAACCCCAGGCACC |
| | AGCGAGTCTGCCACACCAGAATCATCTGGCGGTAGCTCCGGCGGCAGCGACAAGAAGTATT |
| | CTATCGGACTGGCCATCGGCACCAACTCTGTTGGATGGGCCGTGATCACCGACGAGTACAA |
| | GGTGCCCAGCAAGAAATTCAAGGTGCTGGGCAACACCGACAGGCACAGCATCAAGAAGAAC |
| | CTGATCGGCGCACTGCTGTTCGACTCTGGCGAAACAGCCGAGGCCACCAGACTGAAGAGAA |
| | CAGCCCGCAGACGGTACACCAGAAGAAAGAACCGGATCTGCTACCTCCAAGAGATCTTCAG |
| | CAACGAGATGGCCAAGGTGGACGACAGCTTCTTCCACAGACTGGAAGAGTCCTTCCTGGTG |
| | GAAGAGGACAAGAAGCACGAGAGACACCCCATCTTCGGCAACATCGTGGACGAGGTGGCCT |
| | ACCACGAGAAGTACCCCACCATCTACCACCTGAGAAAGAAACTGGTGGACAGCACCGACAA |
| | GGCCGACCTGAGACTGATCTATCTGGCCCTGGCTCACATGATCAAGTTCCGGGGCCACTTC |
| | CTGATCGAGGGCGACCTGAATCCTGACAACAGCGACGTGGACAAGCTGTTCATCCAGCTGG |
| | TGCAGACCTACAACCAGCTGTTCGAGGAAAACCCCATCAACGCCAGCGGAGTGGATGCCAA |
| | GGCCATCCTGTCTGCCAGACTGAGCAAGAGCAGACGGCTGGAAAATCTGATCGCCCAGCTG |
| | CCTGGCGAGAAGAAGAATGGCCTGTTCGGCAACCTGATTGCCCTGAGCCTGGGCCTGACAC |
| | CTAACTTCAAGAGCAACTTCGACCTGGCCGAGGACGCCAAACTGCAGCTGAGCAAGGACAC |
| | CTACGACGACGACCTGGACAATCTGCTGGCCCAGATCGGCGATCAGTACGCCGACTTGTTT |
| | CTGGCCGCCAAGAATCTGAGCGACGCCATCCTGCTGTCCGACATCCTGAGAGTGAACACCG |
| | AGATCACCAAGGCACCTCTGAGCGCCTCTATGGTCAAGAGATACGACGAGCACCACCAGGA |
| | TCTGACCCTGCTGAAGGCCCTCGTTAGACAGCAGCTGCCAGAGAAGTACAAAGAGATTTTC |
| | TTCGACCAGAGCAAGAACGGCTACGCCGGCTACATTGATGGCGGAGCCAGCCAAGAGGAAT |
| | TCTACAAGTTCATCAAGCCCATCCTCGAGAAGATGGACGGCACCGAGGAACTGCTGGTCAA |
| | GCTGAACAGAGAGGACCTGCTGAGAAAGCAGAGAACCTTCGACAACGGCATCATCCCTCAC |
| | CAGATCCACCTGGGAGAACTGCACGCCATTCTGCGGAGACAAGGGGACTTTTACCCATTCC |
| | TGAAGGACAACCGGGAAAAGATCGAGAAAATCCTGACCTTCAGGATCCCCTACTACGTGGG |
| | ACCACTGGCCAGAGGCAATAGCAGATTCGCCTGGATGACCAGAAAGAGCGAGGAAACCATC |
| | ACTCCCTGGAACTTCGAGGAAGTGGTGGACAAGGGCGCCAGCGCTCAGTCCTTCATCGAGC |
| | GGATGACCAACTTCGATAAGAACCTGCCTAACGAGAAGGTGCTGCCCAAGCACAGCCTGCT |
| | GTACGAGTACTTCACCGTGTACAACGAGCTGACCAAAGTGAAATACGTGACCGAGGGAATG |
| | AGAAAGCCCGCCTTTCTGAGCGGCGAGCAGAAAAAGGCCATCGTGGATCTGCTGTTCAAGA |
| | CCAACCGGAAAGTGACCGTGAAGCAGCTGAAAGAGGACTACTTCAAGAAAATCGAGTGCTT |
| | CGACAGCGTCGAGATCTCCGGCGTGGAAGATCGGTTCAATGCCAGCCTGGGCACATACCAC |
| | GATCTGCTGAAAATTATCAAGGACAAGGACTTCCTGGACAACGAAGAGAACGAGGACATCC |
| | TTGAGGACATCGTGCTGACACTGACCCTGTTTGAGGACAGAGAGATGATCGAGGAACGGCT |
| | GAAAACATACGCCCACCTGTTCGACGACAAAGTGATGAAGCAACTGAAGCGGCTGAGATAC |
| | ACCGGCTGGGGCAGACTGTCTCGGAAGCTGATCAACGGCATCCGGGATAAGCAGTCCGGCA |
| | AGACCATCCTGGACTTTCTGAAGTCCGACGGCTTCGCCAACAGAAACTTCATGCAGCTGAT |
| | TCACGACGACAGCCTCACCTTCAAAGAGGATATCCAGAAAGCCCAGGTGTCCGGCCAGGGC |
| | GATTCTCTGCATGAGCACATTGCCAACCTGGCCGGCTCTCCCGCCATTAAGAAAGGCATCC |
| | TGCAGACAGTGAAGGTGGTGGACGAGCTTGTGAAAGTGATGGGCGGACACAAGCCCGAGAA |
| | CATCGTGATCGAAATGGCCAGAGAGAACCAGACCACACAGAAGGGACAGAAGAACAGCCGC |
| | GAGAGAATGAAGCGGATCGAAGAGGGCATCAAAGAGCTGGGCAGCCAGATCCTGAAAGAAC |
| | ACCCCGTGGAAAACACCCAGCTGCAGAACGAGAAGCTGTACCTGTACTACCTGCAGAATGG |
| | ACGGGATATGTACGTGGACCAAGAGCTGGACATCAACAGACTGTCCGACTACGATGTGGAC |
| | CATATCGTGCCCCAGTCTTTTCTGAAGGACGACTCCATCGACAACAAGGTCCTGACCAGAT |
| | CCGACAAGAATCGGGGCAAGAGCGACAACGTGCCCTCCGAAGAGGTGGTCAAGAAGATGAA |
| | GAACTACTGGCGACAGCTGCTGAACGCCAAGCTGATTACCCAGCGGAAGTTCGACAATCTG |
| | ACCAAGGCCGAAAGAGGCGGCCTGAGCGAACTGGATAAGGCCGGCTTCATCAAGAGACAGC |
| | TGGTGGAAACCCGGCAGATCACAAAGCACGTGGCACAGATTCTGGACTCTCGGATGAACAC |
| | TAAGTACGACGAGAACGACAAACTGATCCGCGAAGTGAAAGTCATCACCCTGAAGTCCAAG |
| | CTGGTGTCCGATTTCCGGAAGGATTTCCAGTTCTACAAAGTGCGCGAGATCAACAACTACC |
| | ATCACGCCCACGACGCCTACCTGAATGCCGTTGTTGGAACAGCCCTGATCAAAAAGTACCC |
| | TAAGCTGGAAAGCGAGTTCGTGTACGGCGACTACAAGGTGTACGACGTGCGGAAGATGATC |
| | GCCAAGAGCGAGCAAGAGATTGGCAAGGCAACCGCCAAGTACTTCTTCTACAGCAACATCA |
| | TGAACTTTTTCAAGACAGAGATCACCCTCGCCAACGGCGAGATCAGAAAGCGGCCTCTGAT |
| | CGAGACAAACGGCGAAACCGGCGAGATTGTGTGGGATAAGGGCAGAGACTTTGCCACAGTG |
| | CGGAAAGTGCTGAGCATGCCCCAAGTGAATATCGTGAAGAAAACCGAGGTGCAGACAGGCG |
| | GCTTCAGCAAAGAGTCTATCCTGCCTAAGGGGAACTCCGACAAGCTGATCGCCAGAAAGAA |
| | GGACTGGGACCCCAAGAAATACGGCGGCTTTAACTCTCCCACCGTGGCCTATTCTGTTCTG |
| | GTGGTGGCCAAAGTGGAAAAGGGCAAGTCCAAGAAACTCAAGAGCGTGAAAGAGCTGCTGG |
| | GGATCACCATCATGGAAAGAAGCAGCTTCGAGAAGAATCCGATCGATTTCCTCGAGGCCAA |
| | GGGTTACAAAGAAGTGAAAAAGGACCTGATCATCAAGCTCCCCAAGTACTCCCTGTTCGAG |
| | CTGGAAAACGGCCGGAAGAGAATGCTGGCCTCTGCCGGTGTCCTGCAGAAGGGAAACGAAC |
| | TGGCCCTGCCTAGCAAATATGTGAACTTCCTGTACCTGGCCAGCCACTATGAGAAGCTGAA |
| | GGGCAGCCCCGAGGACAATGAGCAAAAGCAGCTGTTTGTGGAACAGCACAAGCACTACCTG |
| | GACGAGATCATCGAGCAGATCAGCGAGTTTAGCAAGAGAGTGATTCTGGCCGACGCCAATC |
| | TGGACAAAGTGCTGTCCGCCTACAACAAGCACCGGGACAAGCCTATCAGAGAGCAGGCCGA |
| | GAATATCATCCACCTGTTTACCCTGACCAACCTGGGAGCCCCTGCGGCCTTCAAGTACTTT |
| | GACACCACCATCAACAGGAAGCAGTACAACACCACCAAAGAGGTGCTGGACGCCACTCTGA |
| | TCCGCCAGTCTATCACCGGCCTGTACGAGACACGGATCGACCTGTCTCAACTCGGAGGCGA |
| | CGAAGGCGCCGATAAGAGAACCGCCGATGGCTCTGAGTTCGAGAGCCCTAAGAAAAAGCGC |
| | AAAGTGTAG (SEQ ID NO: 490) |

In some cases (e.g., ABE9v2) the mRNA encoding the base editor contains a 5′ untranslated region (UTR) with the nucleotide sequence TAATDKACCACGNDYTTTGAACCTTGCGTAATGDKACGTGGNDYTGTACAGCGTCGCATCAT AAAGTACGACTCACTATAAGGAAATAAGAGAGAAAAGAAGAGTAAGAAGAAATATAAGAGCC ACC (SEQ ID NO: 491) and a 3′ untranslated region (UTR) with the nucleotide sequence TTAATTAAGCTGCCTTCTGCGGGGCTTGCCTTCTGGCCATGCCCTTCTTCTCTCCCTTGCAC CTGTACCTCTTGGTCTTTGAATAAAGCCTGAGTAGGAAGAAAAAAAAAAAAAAAAAAAAAAAA AAAAAAAAAAAAAAAAAAAAAAAAAAAAAAAAAAAAAAAAAAAAAAAAA AAAAAAAAAAAAAAAAAAAAAAAAAAAAAAA (SEQ ID NO: 492). For example, mRNA2518 transcribed from the plasmid pDKL-208 and encoding ABE9v2 had the following nucleotide sequence:

(SEQ ID NO: 493)
TAATDKACCACGNDYTTTGAACCTTGCGTAATGDKACGTGGNDYTGTACAGCGTCGCATCAT
AAAGTACGACTCACTATAAGGAAATAAGAGAGAAAAGAAGAGTAAGAAGAAATATAAGAGCC
ACCATGTCCGAAGTCGAGTTTTCCCATGAGTACTGGATGAGACACGCATTGACTCTCGCAAA
GAGGGCTCGAGATGAACGCGAGGTGCCCGTGGGGGCAGTACTCGTGCTCAACAATCGCGTAA
TCGGCGAAGGTTGGAATAGGGCAATCGGACTCCACGACCCCACTGCACATGCGGAAATCATG
GCCCTTCGACAGGGAGGGCTTGTGATGCAGAATTATCGACTTTATGATGCGACGCTGTACAC
CACGTTTGAACCTTGCGTAATGTGCGCGGGAGCTATGATTCACTCCCGCATTGGACGAGTTG
TATTCGGTGTTCGCAACGCCAAGACGGGTGCCGCAGGTTCACTGATGGACGTGCTGCATCAT
CCAGGCATGAACCACCGGGTAGAAATCACAGAAGGCATATTGGCGGACGAATGTGCGGCGCT
GTTGTGTGACTTTTATCGCATGCCCAGGCGTGTCTTTAACGCCCAGAAAAAAGCACAATCCT
CTATAAACTCCGGCGGAAGCAGCGGAGGATCTTCTGGAAGCGAAACCCCAGGCACCAGCGAG
TCTGCCACACCAGAATCATCTGGCGGTAGCTCCGGCGGCAGCGACAAGAAGTATTCTATCGG
ACTGGCCATCGGCACCAACTCTGTTGGATGGGCCGTGATCACCGACGAGTACAAGGTGCCCA
GCAAGAAATTCAAGGTGCTGGGCAACACCGACAGGCACAGCATCAAGAAGAACCTGATCGGC
GCACTGCTGTTCGACTCTGGCGAAACAGCCGAGGCCACCAGACTGAAGAGAACAGCCCGCAG
ACGGTACACCAGAAGAAAGAACCGGATCTGCTACCTCCAAGAGATCTTCAGCAACGAGATGG
CCAAGGTGGACGACAGCTTCTTCCACAGACTGGAAGAGTCCTTCCTGGTGGAAGAGGACAAG
AAGCACGAGAGACACCCCATCTTCGGCAACATCGTGGACGAGGTGGCCTACCACGAGAAGTA
CCCCACCATCTACCACCTGAGAAAGAAACTGGTGGACAGCACCGACAAGGCCGACCTGAGAC
TGATCTATCTGGCCCTGGCTCACATGATCAAGTTCCGGGGCCACTTCCTGATCGAGGGCGAC
CTGAATCCTGACAACAGCGACGTGGACAAGCTGTTCATCCAGCTGGTGCAGACCTACAACCA
GCTGTTCGAGGAAAACCCCATCAACGCCAGCGGAGTGGATGCCAAGGCCATCCTGTCTGCCA
GACTGAGCAAGAGCAGACGGCTGGAAAATCTGATCGCCCAGCTGCCTGGCGAGAAGAAGAAT
GGCCTGTTCGGCAACCTGATTGCCCTGAGCCTGGGCCTGACACCTAACTTCAAGAGCAACTT
CGACCTGGCCGAGGACGCCAAACTGCAGCTGAGCAAGGACACCTACGACGACGACCTGGACA
ATCTGCTGGCCCAGATCGGCGATCAGTACGCCGACTTGTTTCTGGCCGCCAAGAATCTGAGC
GACGCCATCCTGCTGTCCGACATCCTGAGAGTGAACACCGAGATCACCAAGGCACCTCTGAG
CGCCTCTATGGTCAAGAGATACGACGAGCACCACCAGGATCTGACCCTGCTGAAGGCCCTCG
TTAGACAGCAGCTGCCAGAGAAGTACAAAGAGATTTTCTTCGACCAGAGCAAGAACGGCTAC
GCCGGCTACATTGATGGCGGAGCCAGCCAAGAGGAATTCTACAAGTTCATCAAGCCCATCCT
CGAGAAGATGGACGGCACCGAGGAACTGCTGGTCAAGCTGAACAGAGAGGACCTGCTGAGAA
AGCAGAGAACCTTCGACAACGGCATCATCCCTCACCAGATCCACCTGGGAGAACTGCACGCC
ATTCTGCGGAGACAAGGGGACTTTTACCCATTCCTGAAGGACAACCGGGAAAAGATCGAGAA
AATCCTGACCTTCAGGATCCCCTACTACGTGGGACCACTGGCCAGAGGCAATAGCAGATTCG
CCTGGATGACCAGAAAGAGCGAGGAAACCATCACTCCCTGGAACTTCGAGGAAGTGGTGGAC
AAGGGCGCCAGCGCTCAGTCCTTCATCGAGCGGATGACCAACTTCGATAAGAACCTGCCTAA
CGAGAAGGTGCTGCCCAAGCACAGCCTGCTGTACGAGTACTTCACCGTGTACAACGAGCTGA
CCAAAGTGAAATACGTGACCGAGGGAATGAGAAAGCCCGCCTTTCTGAGCGGCGAGCAGAAA
AAGGCCATCGTGGATCTGCTGTTCAAGACCAACCGGAAAGTGACCGTGAAGCAGCTGAAAGA
GGACTACTTCAAGAAAATCGAGTGCTTCGACAGCGTCGAGATCTCCGGCGTGGAAGATCGGT
TCAATGCCAGCCTGGGCACATACCACGATCTGCTGAAAATTATCAAGGACAAGGACTTCCTG
GACAACGAAGAGAACGAGGACATCCTTGAGGACATCGTGCTGACACTGACCCTGTTTGAGGA
CAGAGAGATGATCGAGGAACGGCTGAAAACATACGCCCACCTGTTCGACGACAAAGTGATGA
AGCAACTGAAGCGGCTGAGATACACCGGCTGGGGCAGACTGTCTCGGAAGCTGATCAACGGC
ATCCGGGATAAGCAGTCCGGCAAGACCATCCTGGACTTTCTGAAGTCCGACGGCTTCGCCAA
CAGAAACTTCATGCAGCTGATTCACGACGACAGCCTCACCTTCAAAGAGGATATCCAGAAAG
CCCAGGTGTCCGGCCAGGGCGATTCTCTGCATGAGCACATTGCCAACCTGGCCGGCTCTCCC
GCCATTAAGAAAGGCATCCTGCAGACAGTGAAGGTGGTGGACGAGCTTGTGAAAGTGATGGG
CGGACACAAGCCCGAGAACATCGTGATCGAAATGGCCAGAGAGAACCAGACCACACAGAAGG
GACAGAAGAACAGCCGCGAGAGAATGAAGCGGATCGAAGAGGGCATCAAAGAGCTGGGCAGC
CAGATCCTGAAAGAACACCCCGTGGAAAACACCCAGCTGCAGAACGAGAAGCTGTACCTGTA
CTACCTGCAGAATGGACGGGATATGTACGTGGACCAAGAGCTGGACATCAACAGACTGTCCG
ACTACGATGTGGACCATATCGTGCCCCAGTCTTTTCTGAAGGACGACTCCATCGACAACAAG
GTCCTGACCAGATCCGACAAGAATCGGGGCAAGAGCGACAACGTGCCCTCCGAAGAGGTGGT
CAAGAAGATGAAGAACTACTGGCGACAGCTGCTGAACGCCAAGCTGATTACCCAGCGGAAGT
TCGACAATCTGACCAAGGCCGAAAGAGGCGGCCTGAGCGAACTGGATAAGGCCGGCTTCATC
AAGAGACAGCTGGTGGAAACCCGGCAGATCACAAAGCACGTGGCACAGATTCTGGACTCTCG
GATGAACACTAAGTACGACGAGAACGACAAACTGATCCGCGAAGTGAAAGTCATCACCCTGA
AGTCCAAGCTGGTGTCCGATTTCCGGAAGGATTTCCAGTTCTACAAAGTGCGCGAGATCAAC
AACTACCATCACGCCCACGACGCCTACCTGAATGCCGTTGTTGGAACAGCCCTGATCAAAAA
GTACCCTAAGCTGGAAAGCGAGTTCGTGTACGGCGACTACAAGGTGTACGACGTGCGGAAGA
TGATCGCCAAGAGCGAGCAAGAGATTGGCAAGGCAACCGCCAAGTACTTCTTCTACAGCAAC
ATCATGAACTTTTTCAAGACAGAGATCACCCTCGCCAACGGCGAGATCAGAAAGCGGCCTCT
GATCGAGACAAACGGCGAAACCGGCGAGATTGTGTGGGATAAGGGCAGAGACTTTGCCACAG
TGCGGAAAGTGCTGAGCATGCCCCAAGTGAATATCGTGAAGAAAACCGAGGTGCAGACAGGC
GGCTTCAGCAAAGAGTCTATCCTGCCTAAGGGGAACTCCGACAAGCTGATCGCCAGAAAGAA
GGACTGGGACCCCAAGAAATACGGCGGCTTTAACTCTCCCACCGTGGCCTATTCTGTTCTGG
TGGTGGCCAAAGTGGAAAAGGGCAAGTCCAAGAAACTCAAGAGCGTGAAAGAGCTGCTGGGG
ATCACCATCATGGAAAGAAGCAGCTTCGAGAAGAATCCGATCGATTTCCTCGAGGCCAAGGG
TTACAAAGAAGTGAAAAAGGACCTGATCATCAAGCTCCCCAAGTACTCCCTGTTCGAGCTGG
AAAACGGCCGGAAGAGAATGCTGGCCTCTGCCGGTGTCCTGCAGAAGGGAAACGAACTGGCC
CTGCCTAGCAAATATGTGAACTTCCTGTACCTGGCCAGCCACTATGAGAAGCTGAAGGGCAG
CCCCGAGGACAATGAGCAAAAGCAGCTGTTTGTGGAACAGCACAAGCACTACCTGGACGAGA
TCATCGAGCAGATCAGCGAGTTTAGCAAGAGAGTGATTCTGGCCGACGCCAATCTGGACAAA
GTGCTGTCCGCCTACAACAAGCACCGGGACAAGCCTATCAGAGAGCAGGCCGAGAATATCAT
CCACCTGTTTACCCTGACCAACCTGGGAGCCCCTGCGGCCTTCAAGTACTTTGACACCACCA
TCAACAGGAAGCAGTACAACACCACCAAAGAGGTGCTGGACGCCACTCTGATCCGCCAGTCT
ATCACCGGCCTGTACGAGACACGGATCGACCTGTCTCAACTCGGAGGCGACGAAGGCGCCGA
TAAGAGAACCGCCGATGGCTCTGAGTTCGAGAGCCCTAAGAAAAAGCGCAAAGTGTAGTTAA
TTAAGCTGCCTTCTGCGGGGCTTGCCTTCTGGCCATGCCCTTCTTCTCTCCCTTGCACCTGT
ACCTCTTGGTCTTTGAATAAAGCCTGAGTAGGAAGAAAAAAAAAAAAAAAAAAAAAAAAAAA
AAAAAAAAAAAAAAAAAAAAAAAAAAAAAAAAAAAAAAAAAAAAAAAAAAAAAAAAAAAAAA
AAAAAAAAAAAAAAAAAAAAAAAAAAA.

### General Methods

All cloning was conducted via USER enzyme (New England Biolabs) cloning methods (see Geu-Flores et al., USER fusion: a rapid and efficient method for simultaneous fusion and cloning of multiple PCR products. Nucleic Acids Res 35, e55, doi:10.1093/nar/gkm106 (2007)) and templates for PCR amplification were purchased as bacterial or mammalian codon optimized gene fragments (GeneArt). Vectors created were transformed into Mach T1^{R} Competent Cells (ThermoFisher Scientific) and maintained at −80 C for long-term storage. All primers used in this work were purchased from Integrated DNA Technologies and PCRS were carried out using either Phusion U DNA Polymerase Green MultiPlex PCR Master Mix (ThermoFisher) or Q5 Hot Start High-Fidelity 2× Master Mix (New England Biolabs). All plasmids used in this work were freshly prepared from 50 mL of Mach1 culture using ZymoPURE Plasmid Midiprep (Zymo Research Corporation) which involves an endotoxin removal procedure. Molecular biology grade, Hyclone water (GE Healthcare Life Sciences) was used in all assays, transfections, and PCR reactions to ensure exclusion of DNAse activity.

Nucleotide sequences of sgRNAs used for Hek293T mammalian cell transfection are provided in Table 10 below. The 20-nt target protospacer is shown in bold font. When a target DNA sequence did not start with a ‘G,’ a ‘G’ was added to the 5′ end of the primer since it has been established that the human U6 promoter prefers a ‘G’ at the transcription start site (see Cong, L. et al., Multiplex genome engineering using CRISPR/Cas systems. Science 339, 819-823, doi:10.1126/science.1231143 (2013)). The pFYF sgRNA plasmid described previously was used as a template for PCR amplification.

TABLE 10
Sequences of sgRNAs used for Hek293T mammalian cell transfection.

| | | SEQ ID | Cas9 and | |
|---|---|---|---|---|
| Site | Spacer Sequence | NO. | Scaffold | PAM |
| 1 | GAACACAAAGCAUAGACUGC | 494 | S. pyogenes | NGG |
| 2 | GGGAAAGACCCAGCAUCCGU | 495 | S. pyogenes | NGG |
| 3 | GCUCCCAUCACAUCAACCGG | 496 | S. pyogenes | NGG |
| 4 | GGUGAGUGAGUGUGUGCGUG | 497 | S. pyogenes | NGG |
| 5 | GGCUUCAGGUUCUAAAUGAG | 498 | S. pyogenes | NGG |
| 6 | GCAGAGAGUCGCCGUCUCCA | 499 | S. pyogenes | NGG |
| 7 | GUGUAAGACCUCAAAAGCAC | 500 | S. pyogenes | NGG |
| 8 | GAUGAGAAGGAGAAGUUCUU | 501 | S. pyogenes | NGG |
| 9 | GAGGACAAAGUACAAACGGC | 502 | S. pyogenes | AGA |
| 10 | GCCACCACAGGGAAGCUGGG | 503 | S. pyogenes | TGA |
| 11 | GCUCUCAGGCCCUGUCCGCA | 504 | S. pyogenes | CGT |
| 12 | GAGCAAAUACCAGAGAUAAG | 505 | S. pyogenes | AGA |
| 13 | GAUCAGGAAAUAGAGCCACA | 506 | S. pyogenes | GGC |
| 14 | GCCCAUCCCUGAGUCCAGCG | 507 | S. pyogenes | AGC |
| 15 | GAACACGAAGACAUCUGAAGGUA | 508 | S. aureus | TTGAAT |
| 16 | GAUUUACAGCCUGGCCUUUGGGG | 509 | S. aureus | TCGGGT |
| 17 | GGAGAGAAAGAGAAGUUGAUUG | 510 | S. aureus | ATGGGT |
| 18 | GAGGGUGAGGGAUGAGAUAAUG | 511 | S. aureus | ATGAGT |
| 19 | GGUGGAGGAGGGUGCAUGGGGU | 512 | S. aureus | CAGAAT |
| 20 | GCUGUUGCAUGAGGAAAGGGAC | 513 | S. aureus | TAGAGT |
| HEK2 | GAACACAAAGCAUAGACUGC | 494 | S. pyogenes | CGG |
| HEK3 | GGCCCAGACUGAGCACGUGA | 515 | S. pyogenes | TGG |
| HEK4 | GGCACUGCGGCUGGAGGUGG | 516 | S. pyogenes | GGG |
| LDLR | GCAGAGCACUGGAAUUCGUCA | 517 | S. pyogenes | GGG |

sgRNA scaffold sequences referenced in Table 10 were as follows:

S. pyogenes:
(SEQ ID NO: 319)
GUUUUAGAGCUAGAAAUAGCAAGUUAAAAUAAGGCUAGUCCGUUAUCAA
CUUGAAAAAGUGGCACCGAGUCGGUGC
S. aureus:
(SEQ ID NO: 320)
GUUUUAGUACUCUGUAAUGAAAAUUACAGAAUCUACUAAAACAAGGCAA
AAUGCCGUGUUUAUCUCGUCAACUUGUUGGCGAGA

### Generation of Input Bacterial TadA* Libraries for Directed Evolution

The TadA*8.0 library was designed to encode all 20 amino acids at each amino acid position in the TadA*7.10 open reading frame (Gaudelli, N. M. et al., Programmable base editing of A*T to G*C in genomic DNA without DNA cleavage. Nature 551, 464-471, doi:10.1038/nature24644 (2017)). Each TadA*8.0 library member contained about 1-2 new coding mutations and was chemically synthesized and purchased from Ranomics Inc (Toronto, Canada). The TadA*8.0 library was PCR amplified with Phusion U Green MultiPlex PCR Master Mix and USER-assembled into a bacterial vector optimized for ABE directed evolution (Gaudelli, N. M. et al., Programmable base editing of A*T to G*C in genomic DNA without DNA cleavage. Nature 551, 464-471, doi:10.1038/nature24644 (2017)).

### Bacterial Evolution of TadA Variants

Directed evolution of ABE containing the TadA*8 library was conducted as previously described (Gaudelli, N. M. et al., Programmable base editing of A*T to G*C in genomic DNA without DNA cleavage. Nature 551, 464-471, doi:10.1038/nature24644 (2017)) with the following changes: i) E. coli 10 betas (New England Biolabs) were used as the evolution host; and ii) survival on kanamycin relied on correction of three genetic inactivating components (e.g. survival required reversion of two stop mutations and one active site mutation in kanamycin). The kanamycin resistance gene sequence contains selection mutations for ABE8 evolution. After overnight co-culturing of selection plasmid and editor in 10 beta host cells, the library cultures were plated on 2×YT-agar medium supplemented with plasmid maintenance antibiotic and increasing concentrations of selection antibiotic, kanamycin (64-512 μg/mL). Bacteria were allowed to grow for 1 day and the TadA*8 portion of the surviving clones were Sanger sequenced after enrichment. Identified TadA*8 mutations of interest were then were then incorporated into mammalian expression vector via USER assembly.

### General Mammalian Culture Conditions

Cells were cultured at 37° C. with 5% CO₂. HEK293T cells [CLBTx013, American Type Cell Culture Collection (ATCC)] were cultured in Dulbecco's modified Eagles medium plus Glutamax (10566-016, Thermo Fisher Scientific) with 10% (v/v) fetal bovine serum (A31606-02, Thermo Fisher Scientific).

Hek293T Plasmid Transfection and gDNA Extraction

HEK293T cells were seeded onto 48-well well Poly-D-Lysine treated BioCoat plates (Corning) at a density of 35,000 cells/well and transfected 18-24 hours after plating. Cells were counted using a NucleoCounter NC-200 (Chemometec). To these cells were added 750 ng of base editor or nuclease control, 250 ng of sgRNA, and 10 ng of GFP-max plasmid (Lonza) diluted to 12.5 μL total volume in Opti-MEM reduced serum media (ThermoFisher Scientific). The solution was combined with 1.5 μL of Lipofectamine 2000 (ThermoFisher) in 11 μL of Opti-MEM reduced serum media and left to rest at room temperature for 15 min. The entire 25 μL mixture was then transferred to the pre-seeded Hek293T cells and left to incubate for about 120 h. Following incubation, media was aspirated and cells were washed two times with 250 μL of 1×PBS solution (ThermoFisher Scientific) and 100 μL of freshly prepared lysis buffer was added (100 mM Tris-HCl, pH 7.0, 0.05% SDS, 25 μg/mL Proteinase K (Thermo Fisher Scientific). Transfection plates containing lysis buffer were incubated at 37° C. for 1 hour and the mixture was transferred to a 96-well PCR plate and heated at 80° C. for 30 min.

### CD34+ Cell Preparation

Mobilized peripheral blood was obtained from sickle trait individuals and enriched for Human CD34+ HSPCs (Allcells, MOO1F-GCSF/MOZ-2). The CD34+ cells were thawed and put into X-VIVO 10 (Lonza) containing 1% Glutamax (Gibco), 100 ng/mL of TPO (Peprotech), SCF (Peprotech) and Flt-3 (Peprotech) at 48 hours prior to electroporation.

### Electroporation of CD34+ Cells

48 hours post thaw, CD34+ cells were spun down to remove X-VIVO 10 media and washed in MaxCyte buffer (HyClone) with 0.1% HSA (Akron Biotechnologies). The cells were then resuspended in cold MaxCyte buffer at 1,250,000 cells per mL and split into multiple 80 μL aliquots. An mRNA molecule encoding a base editor and a guide polynucleotide were then aliquoted and raised to a total of 20 μL in MaxCyte buffer. The 80 μL of cells was the added into the 20 μL RNA mixture in groups of 2 and loaded into each chamber of an OC100×2 MaxCyte cuvette for electroporation. After receiving the electroporation charge, 100 μL was collected from the chambers and placed in the center of the wells in a 24-well untreated culture plate. The cells recovered for 20 minutes in an incubator (37° C., 5% CO₂). After the 20 minutes recovery, X-VIVO 10 (a hematopoietic cell medium) containing 1% Glutamax, 100 ng/mL of TPO, SCF and Flt-3 was added to the cells for a concentration of 1,000,000 cells per mL. The cells were then left to further recover in an incubator (37° C., 5% CO₂) for 48 hrs.

### Genomic DNA Extraction for CD34+ Cells

At 48 hours following electroporation, an aliquot of cells was post-cultured in X-VIVO 10 media (Lonza) containing 1% Glutamax (Gibco), 100 ng/mL of TPO (Peprotech), SCF (Peprotech) and Flt-3 (Peprotech). At 48 h and 144 h post-culturing, 100,000 cells were collected and spun down. 50 μL of Quick Extract (Lucigen) was added to the resulting cell pellet and the cell mixture was transferred to a 96-well PCR plate (Bio-Rad). The lysate was heated for 15 minutes at 65° C. followed by 10 minutes at 98° C. The cell lysates were stored at −20° C. for later analysis.

### OTHER EMBODIMENTS

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The invention of the disclosure may be related to PCT/US2020/018193, filed Feb. 13, 2020, or to PCT/US19/31897, filed May 11, 2019, the disclosures of which are incorporated herein by reference in their entireties for all purposes

What is claimed:

1. A method for treating a hemoglobinopathy in a subject in need thereof, the method comprising:
a) contacting an isolated hematopoietic stem cell or progenitor thereof with two or more guide polynucleotides, or one or more polynucleotides encoding the guide polynucleotides, and a base editor comprising a nucleic acid programmable DNA binding protein (napDNAbp) and an adenosine deaminase, or one or more polynucleotides encoding the base editor, wherein the adenosine deaminase comprises a combination of alterations relative to the following sequence of TadA*7.10 and has at least 85% sequence identity to the following sequence of TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), wherein the combination of alterations comprises:
i) F149Y and V82T; or
ii) F149Y, T166I, and/or D167N, and the alteration V82T; and
wherein the two or more guide polynucleotides target the base editor to effect an alteration to a beta globin polynucleotide (HBB) that results in expression of a beta globin polypeptide having an alanine at position 6 (Hb G-Makassar), and wherein the two or more guide polynucleotides target the base editor to effect an alteration in a cluster of differentiation (CD117) polynucleotide that results in expression of a CD117 polypeptide with reduced binding to an antibody that selectively binds a wild type CD117 polypeptide, thereby generating an edited cell; and
(b) wherein the cell is in the subject or the method further comprises administering the edited cell to the subject.

2. The method of claim 1, wherein the combination of alterations comprises or further comprises:
I76Y, V82T, Y123H, Y147R, F149Y;
Q154R or F149Y, T166I, and D167N;
I76Y, Y123H, Y147D, and Q154R; and/or
I76Y, V82T, Y123H, Y147D, F149Y, Q154R, T166I, and D167N

3. The method of claim 1, wherein the adenosine deaminase comprises the following amino acid sequence: SEVEFSHEYWMRHALTLAKRARDEREVPVGAVLVLNNRVIGEGWNRAIGLHDPTAHAEIMAL RQGGLVMQNYRLYDATLYTTFEPCVMCAGAMIHSRIGRVVFGVRNAKTGAAGSLMDVLHHPG MNHRVEITEGILADECAALLCDFYRMPRRVFNAQKKAQSSIN (SEQ ID NO: 519); and
wherein the two or more guide polynucleotides comprise a spacer nucleotide sequence selected from the group consisting of: UUCUCCACAGGAGUCAGGUG (SEQ ID NO: 445); ACUUCUCCACAGGAGUCAGG (SEQ ID NO: 446); GACUUCUCCACAGGAGUCAGG (SEQ ID NO: 447); CUUCUCCACAGGAGUCAGG (SEQ ID NO: 448); CUUCUCCACAGGAGUCAGAU (SEQ ID NO: 449); ACUUCUCCACAGGAGUCAGAU (SEQ ID NO: 450); GACUUCUCCACAGGAGUCAGAU (SEQ ID NO: 451); UCUGACUCCUGUGGAGAAGUCU (SEQ ID NO: 452); AGACUUCUCCACAGGAGUCAGA (SEQ ID NO: 453); and UCCACAGGAGUCAGAUGCAC (SEQ ID NO: 454); or a spacer sequence selected from those listed in Table 2; and, optionally, a scaffold with the following nucleotide sequence:
(SEQ ID NO: 317)
GUUUUAGAGCUAGAAAUAGCAAGUUAAAAUAAGGCUAGUCCGUUAUCAA
CUUGAAAAAGUGGCACCGAGUCGGUGCUUUU.

4. The method of 1, wherein the two or more guide polynucleotides comprise a spacer nucleotide sequence selected from the group consisting of:
(SEQ ID NO 660; CC128)
AAAUAUAAUAGCUGGCAUCA;
(SEQ ID NO: 722; CC200)
AUAAUAGCUGGCAUCACGGU;
(SEQ ID NO: 723; gRNA889)
CCACUAGCUUUCCAAACGGU;
(SEQ ID NO: 724; gRNA908)
GCUGAACUGAUAGUCAACGU;
(SEQ ID NO: 725; gRNA918)
UUUGACAAAGCCCGGAUCAG;
(SEQ ID NO: 726; gRNA923)
UGAAAGUGAGGCCAGGUACU;
(SEQ ID NO: 727; gRNA928)
AAACAGUCAGGUGAGUGAAU;
(SEQ ID NO: 728; gRNA929)
AACUACAGGAGAAAUAUAAU;
and
(SEQ ID NO: 729; gRNA944)
GAUUAAAAGGCACCGAAGGA.

5. The method of claim 1, wherein the two or more guide polynucleotides comprise a scaffold with a sequence selected from the group consisting of: GUUUUAGAGCUAGAAAUAGCAAGUUAAAAUAAGGCUAGUCCGUUAUCAACUUGAAAAAGUGG CACCGAGUCGGUGCmUsmUsmUsU (SEQ ID NO: 521); GUUUUAGAmGmCmCmGmGmCmGmGmAmAmAmCmGmCmCmGmGmCAAGUUAAAAUAAGGCUAG UCCGUUAmUmCAAmCmUmUGGACUUCGGUCCmAmAmGUGGmCmAmCmCmGmAmGmUmCmGmG mUmGmCmUsmUsmUsmU (SEQ ID NO: 522); mNsmNsmNsNNNNNNNNNNNNNNNNNGUUUUAGAGCUAGAAAUAGCAAGUUAAAAUAAGGCU AGUCCGUUAUCAACUUGAAAAAGUGGCACCGAGUCGGUGCmUsmUsmUsU (SEQ ID NO: 443); and mNsmNsmNsNNNNNNNNNNNNNNNNNGUUUUAGAmGmCmCmGmGmCmGmGmAmAmAmCmGmC mCmGmGmCAAGUUAAAAUAAGGCUAGUCCGUUAmUmCAAmCmUmUGGACUUCGGUCCmAmAm GUGGmCmAmCmCmGmAmGmUmCmGmGmUmGmCmUsmUsmUsmU (SEQ ID NO: 444), wherein “N” represents any nucleotide, “mN” indicates a 2′-OMe modification of the nucleotide “N”, and “Ns” indicates that the nucleotide “N” is linked to the following nucleotide by a phosphorothioate (PS).

6. A cell produced by the method of claim 1.

7. A pharmaceutical composition comprising an effective amount of the cell of claim 6.

8. An adenosine deaminase polypeptide comprising a combination of alterations relative to the following sequence of TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), wherein the combination of alterations comprises F149Y and V82T, and wherein the adenosine deaminase has at least 85% sequence identity to TadA*7.10.

9. The adenosine deaminase polypeptide of claim 8, wherein the combination of alterations further comprises an alteration selected from the group consisting of 176Y, Y123H, Y147R, and Q154R.

10. An adenosine deaminase polypeptide comprising the following amino acid sequence: SEVEFSHEYWMRHALTLAKRARDEREVPVGAVLVLNNRVIGEGWNRAIGLHDPTAHAEIMAL RQGGLVMQNYRLYDATLYTTFEPCVMCAGAMIHSRIGRVVFGVRNAKTGAAGSLMDVLHHPG MNHRVEITEGILADECAALLCRFYRMPRRVFNAQKKAQSSTD (SEQ ID NO: 518); or
comprising a combination of alterations to the following sequence of TadA*7.10: MSEVEFSHEYWMRHALTLAKRARDEREVPVGAVLVLNNRVIGEGWNRAIGLHDPTAHAEIMA LRQGGLVMQNYRLIDATLYVTFEPCVMCAGAMIHSRIGRVVFGVRNAKTGAAGSLMDVLHYP GMNHRVEITEGILADECAALLCYFFRMPRQVFNAQKKAQSSTD (SEQ ID NO: 1), wherein the combination of alterations comprises F149Y, T166I, and/or D167N, and the alteration V82T, and wherein the adenosine deaminase has at least 85% sequence identity to TadA*7.10.

11. The adenosine deaminase polypeptide of claim 10, wherein the combination of alterations further comprises the following additional alterations: I76Y, Y123H, Y147D, and Q154R.

12. A base editor comprising a nucleic acid programmable DNA binding protein (napDNAbp) and the adenosine deaminase of claim 10.

13. A base editor comprising: a nucleic acid programmable DNA binding protein (napDNAbp) and an adenosine deaminase, wherein the adenosine deaminase comprises a combination of alterations relative to TadA*7.10:
(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD,
wherein the combination of alterations comprises I76Y, V82T, Y123H, Y147R, F149Y, and Q154R, and wherein the adenosine deaminase has at least 85% sequence identity to TadA*7.10; or
the adenosine deaminase comprises a combination of alterations relative to TadA*7.10:
(SEQ ID NO: 1)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSR
IGRVVFGVRNAKTGAAGSLMDVLHYPGMNHRVEITEGILADECAALLCY
FFRMPRQVFNAQKKAQSSTD,
wherein the combination of alterations comprises I76Y, V82T, Y123H, Y147D, F149Y, Q154R, T166I, and D167N, and wherein the adenosine deaminase has at least 85% sequence identity to TadA*7.10.

14. A polynucleotide encoding the adenosine deaminase polypeptide of claim 10.

15. A method of altering a nucleobase of a polynucleotide, the method comprising contacting the polynucleotide with the base editor of claim 10, thereby altering a nucleobase of the polynucleotide.

16. A base editor system comprising the base editor of claim 13, or one or more polynucleotides encoding the base editor, and a guide polynucleotide, or a polynucleotide encoding the guide polynucleotide, that targets the base editor to effect an alteration to a polynucleotide associated with a genetic disorder.

17. A base editor system comprising the base editor of claim 13, or a polynucleotide encoding the base editor, and two or more guide polynucleotides, or one or more polynucleotides encoding the two or more guide polynucleotides, wherein one of the guide polynucleotides targets the base editor to effect an alteration to a beta globin polynucleotide (HBB) that results in expression of a beta globin polypeptide having an alanine at position 6 (Hb G-Makassar), and another of the guide polynucleotides targets the base editor to effect an alteration in a cluster of differentiation (CD117) polynucleotide that results in expression of a CD117 polypeptide with reduced binding to an antibody that selectively binds a wild type CD117 polypeptide.

18. The base editor system of claim 17, wherein:
a) one of the guide polynucleotides comprises a spacer sequence selected from the group consisting of:
(SEQ ID NO: 445)
UUCUCCACAGGAGUCAGGUG;
(SEQ ID NO: 446)
ACUUCUCCACAGGAGUCAGG;
(SEQ ID NO: 447)
GACUUCUCCACAGGAGUCAGG;
(SEQ ID NO: 448)
CUUCUCCACAGGAGUCAGG;
(SEQ ID NO: 449)
CUUCUCCACAGGAGUCAGAU;
(SEQ ID NO: 450)
ACUUCUCCACAGGAGUCAGAU;
(SEQ ID NO: 451)
GACUUCUCCACAGGAGUCAGAU;
(SEQ ID NO: 452)
UCUGACUCCUGUGGAGAAGUCU;
(SEQ ID NO: 453)
AGACUUCUCCACAGGAGUCAGA;
and
(SEQ ID NO: 454)
UCCACAGGAGUCAGAUGCAC;
b) another of guide polynucleotides comprises a spacer sequence selected from the group consisting of:
(SEQ ID NO 660; CC128)
AAAUAUAAUAGCUGGCAUCA;
(SEQ ID NO: 722; CC200)
AUAAUAGCUGGCAUCACGGU;
(SEQ ID NO: 723; gRNA889)
CCACUAGCUUUCCAAACGGU;
(SEQ ID NO: 724; gRNA908)
GCUGAACUGAUAGUCAACGU;
(SEQ ID NO: 725; gRNA918)
UUUGACAAAGCCCGGAUCAG;
(SEQ ID NO: 726; gRNA923)
UGAAAGUGAGGCCAGGUACU;
(SEQ ID NO: 727; gRNA928)
AAACAGUCAGGUGAGUGAAU;
(SEQ ID NO: 728; gRNA929)
AACUACAGGAGAAAUAUAAU;
and
(SEQ ID NO: 729; gRNA944)
GAUUAAAAGGCACCGAAGGA.

19. A kit suitable for use in the method of any one of the above claims, wherein the kit comprises the base editor of claim 13, or a polynucleotide encoding the base editor.

20. The base editor of claim 13, wherein the base editor comprises the following amino acid sequence:
(SEQ ID NO: 524)
MSEVEFSHEYWMRHALTLAKRARDEREVPVGAVLVLNNRVIGEGWNRAIGLHDPTAHAEIMA
LRQGGLVMQNYRLYDATLYTTFEPCVMCAGAMIHSRIGRVVFGVRNAKTGAAGSLMDVLHHP
GMNHRVEITEGILADECAALLCRFYRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMVKRYDEHHQDLTLLKALVRQQLPEKYKEIFFDQSKNGYA
GYIDGGASQEEFYKFIKPILEKMDGTEELLVKLNREDLLRKQRTFDNGIIPHQIHLGELHAI
LRRQGDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRLRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGGHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKGNSDKLIARKKDWDPKKYGGFNSPTVAYSVLVVAKVEKGKSKKLKSVKELLGI
TIMERSSFEKNPIDFLEAKGYKEVKKDLIIKLPKYSLFELENGRKRMLASAGVLQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTINRKQYNTTKEVLDATLIRQSI
TGLYETRIDLSQLGGDEGADKRTADGSEFESPKKKRKV;
wherein the base editor comprises the following amino acid sequence:
(SEQ ID NO: 525)
MSEVEFSHEYWMRHALTLAKRARDEREVPVGAVLVLNNRVIGEGWNRAIGLHDPTAHAEIMA
LRQGGLVMQNYRLYDATLYTTFEPCVMCAGAMIHSRIGRVVFGVRNAKTGAAGSLMDVLHHP
GMNHRVEITEGILADECAALLCDFYRMPRRVFNAQKKAQSSINSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMVKRYDEHHQDLTLLKALVRQQLPEKYKEIFFDQSKNGYA
GYIDGGASQEEFYKFIKPILEKMDGTEELLVKLNREDLLRKQRTFDNGIIPHQIHLGELHAI
LRRQGDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRLRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGGHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKGNSDKLIARKKDWDPKKYGGFNSPTVAYSVLVVAKVEKGKSKKLKSVKELLGI
TIMERSSFEKNPIDFLEAKGYKEVKKDLIIKLPKYSLFELENGRKRMLASAGVLQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTINRKQYNTTKEVLDATLIRQSI
TGLYETRIDLSQLGGDEGADKRTADGSEFESPKKKRKV.
